(19) United States
(12) Patent Application Publication
(10) Pub. No.: US 2015/0119273 A1
(43) Pub. Date: Apr. 30, 2015

(54) Chronic Traumatic Encephalopathy in Blast-Exposed Individuals

(71) Applicants: Trustees of Boston University, Boston, MA (US); United States Government as Represented by the Department of Veterans Affairs, Washington, DC (US)

(72) Inventors: Lee E. Goldstein, Newton, MA (US); Ann C. Mckee, Wayland, MA (US); Neil W. Kowall, Brookline, MA (US)

(21) Appl. No.: 14/398,099

(22) PCT Filed: May 16, 2013
(86) PCT No.: PCT/US13/41377

§ 371 Date: Oct. 30, 2014

Related U.S. Application Data
(60) Provisional application No. 61/647,842, filed on May 16, 2012.

(51) Int. Cl. G01N 33/68

(52) U.S. Cl. CPC G01N 33/6896; G01N 2440/14; G01N 2800/50; G01N 2800/2828; G01N 2333/4709; G01N 2800/2871
USPC 506/9; 435/7.92; 435/7.1; 436/86; 436/501; 2/468

(57) ABSTRACT

The invention is based on the surprising discovery that as few as one episode of blast exposure increases the risk of CTE. Blast exposure is associated with chronic traumatic encephalopathy, impaired neuronal function, and persistent cognitive deficits in blast-exposed military veterans and experimental animals. Early diagnosis and assessment of risk permits physicians to prescribe treatment to reduce or slow progression of impairment before the onset of overt symptoms that become apparent decades after an initial insult or trauma to brain tissue. The invention provides methods and compositions for diagnosis and prognosis of individuals at risk of long term complications related to blast injury or concussive injury.

### RELATED APPLICATIONS

This application is a national stage application, filed under 35 U.S.C. §371, of International Application No. PCT/US2013/041377, filed May 16, 2013 which claims the benefit of priority under 35 U.S.C. §119(e) to U.S. Provisional Application No. 61/647,842 filed May 16, 2012; the contents of each of which are incorporated herein by reference in their entireties.

### GOVERNMENT SUPPORT

This invention was made with Government support under The Department of Defense, Contract No.: W911NG-06-2-0040; the VA Foundation, Contract No.: B6796-C; and the National Institutes of Health, Contract No.: P30AG13846. The Government has certain rights in the invention.

### INCORPORATION-BY-REFERENCE OF SEQUENCE LISTING

The contents of the text file named “44262-501N01US_ST25.txt”, which was created on Oct. 30, 2014 and is 31 KB in size, are hereby incorporated by reference in their entireties.

### FIELD OF THE INVENTION

The field of the invention pertains to brain injury.

### BACKGROUND OF THE INVENTION

Blast exposure from conventional and improvised explosive devices (IEDs) affects combatants and civilians in conflict regions around the world. Individuals exposed to explosive blast are at increased risk for traumatic brain injury (TBI) that is often reported as mild. Blast-related TBI represents a neuropsychiatric spectrum disorder that clinically overlaps with chronic traumatic encephalopathy (CTE; a.k.a. “boxer's dementia”), a progressive tau protein-linked neurodegenerative disease associated with repetitive concussive injury in athletes. Neuro-pathological hallmarks of CTE include widespread cortical foci of perivascular tau pathology, disseminated microgliosis and astrocytosis, myelinated axonopathy, and progressive neurodegeneration. Clinical symptoms of CTE include progressive affective lability, irritability, distractability, executive dysfunction, memory disturbances, suicidal ideation, and in advanced cases, cognitive deficits and dementia.

### SUMMARY OF THE INVENTION

The invention is based on the surprising discovery that as few as one episode of blast exposure increases the risk of CTE. Blast exposure is associated with chronic traumatic encephalopathy, impaired neuronal function, and persistent cognitive deficits in blast-exposed military veterans and experimental animals. Early diagnosis and assessment of risk permits physicians to prescribe treatment to reduce or slow progression of impairment before the onset of overt symptoms that become apparent decades after an initial insult or trauma to brain tissue. The invention provides methods and compositions for diagnosis and prognosis of individuals at risk of long term complications related to blast injury or concussive injury. The methods are useful to determine and compute a risk level after acute concussive or subconcussive head injury from blast exposure, impact head injury, acceleration or deceleration head trauma, or other type of single or repeated closed-skull neurotrauma.

Accordingly, a method of determining risk of developing chronic traumatic encephalopathy (CTE) of a subject is carried out by evaluating a CTE-linked neuropathic marker after a first blast injury or concussive injury and before a 2^{nd}, 5^{th}, 10^{th}, 25^{th}, 50^{th}, or 100th blast, subconcussive, or concussive injury. A CTE-linked marker comprises phosphorylated forms of tau protein or tau protein fragments (tau peptides) and/or biomarkers of myelinated axonopathy; microvasculopathy; blood-brain barrier compromise or loss of structural or functional integrity; chronic neuroinflammation and neuroinflammatory mediators, cytokines, and/or peptides; reactive astrocyte and/or microglial products; and or neurodegeneration in the absence of macroscopic tissue damage or hemorrhage. CTE diagnostic markers are evaluated by magnetic resonance imaging, diffusion tensor imaging (dti), positron emission tomography, magnetic resonance imaging and related imaging modalities, magnetic resonance spectroscopy, analysis of cerebrospinal fluid, blood plasma or serum or whole blood.

Evaluation is made at the time point of acute injury or up to 20 years following, e.g., 1, 5, 10, 20, 30, 45, 60 minutes; 1.5, 2, 5, 10, 12, 24 hours; 1.5, 2, 3, 4, 5, 6, days, one week, 2 weeks, 3 weeks; one month, one year, 2, years, 5 years, 10 years, or more acute neurotrauma, e.g., after the blast injury, subconcussive injury, or concussive injury. A blast injury includes an impact injury or exposure to a blast wind. These index metrics are also therefore useful as diagnostic markers of chronic evolving disease. Patients suspected of having neurological damage are screened using the methods and/or long-term monitoring is carried out long after the inciting trauma.

A method of determining risk of developing CTE is carried out by detecting a CTE-linked neuropathic marker, e.g., a marker comprising a microtubule associated tau protein (Tau) or a fragment thereof, in a bodily fluid after at least a first blast injury, subconcussive injury, or concussive injury. Based on the level or concentration of the marker in a bodily fluid, a risk level of developing CTE later in life is computed. In one example, the bodily fluid comprises a blood composition such as plasma or serum. The methods are carried out on whole blood and derivative fractions or components obtained from blood. In another example, the bodily fluid comprises saliva, urine, or cerebrospinal fluid.

A total level of Tau protein or fragment thereof is measured and computed to determine risk or a level of a Tau protein or fragment comprising a phosphorylated amino acid is measured and computed. Both are done to compute a ratio of phosphorylated Tau to total Tau in a given sample of bodily fluid. For example, the Tau protein or fragment thereof comprises a phosphorylated amino acid at positions S202, S396, S404, T181, or T205 as well as other Tau phosphorylation sites and combinations thereof. A lower case “p” prior to the amino acid/location coordinate designates a phosphorylated, e.g., “pS202” denotes phosphorylated serine at position 202. Tau protein, fragments or peptides, modified Tau, and or breakdown products of Tau are evaluated.

To compute a risk of developing CTE or achieving a prognostic indication from calculating the level of biomarker, the concentration of biomarker in a patient sample is compared to a standard of values. For example, a concentration of greater than 0.5±1 pg/ml total Tau protein or fragment thereof in plasma or serum indicates an increased risk or propensity of developing CTE. As was described above, the level is measured at various time points, e.g., acutely—shortly after an incident such as a blast (within minutes to an hour) or in an ongoing fashion, every hour or few hours or every day or days, and ongoing over the lifetime of the affected individual. A concentration of greater than 1 pg/ml total Tau protein in plasma or serum indicates a moderate risk of developing CTE, and a concentration of greater than 5 pg/ml total Tau protein in plasma or serum indicates a severe risk of developing CTE.

The appearance of Tau protein or fragments thereof in the bloodstream is indicative of a danger or risk of developing CTE. Increased levels indicate a greater risk. However, the appearance of phosphorylated Tau indicates an even worse prognosis or even greater risk of developing CTE. Thus, the method further comprises computing a ratio of phosphorylated Tau to total Tau, and wherein an increase in said ratio over time indicated an increased risk of developing CTE. Increased levels and increased ratios indicate that treatment for CTE should be administered. The methods detect pathology much earlier than other methods and thus afford an opportunity for early treatment and intervention—a significant advantage over existing methods.

If Tau and/or pTau are elevated in acute aftermath (minutes to hours), the clinical diagnosis and/or prognosis is bad. If sustained over serial sampling, the prognosis is worse. If levels increase over serial sampling or if phosphorylated tau begins to peak, the prognosis is much worse still. On the other hand, a declining level or absence of phosphorylated tau, indicates a resolving brain injury and a better prognosis than if otherwise. The prognosis pattern is analogous to cardiac enzyme blood levels in the aftermath acute myocardial infarction (AMI, heart attack).

In evaluating tau levels in patient-derived fluids, the following parameters are organized by increasing risk of significant neurologic sequelae such as CTE: elevated total tau protein>normal tau protein levels=1+; presence of phosphorylated tau protein=2+; presence of phosphorylated tau protein in combination with elevated total tau protein=3+ (with increasingly poor prognosis and increasing risk of long-term neurological sequelae with increasing ratio p-tau/total tau: 0-25%+, 25-50%++, 50-75%+++, >75%++++). In addition, the following all of the indicators provide additional poor prognosis and increasing risk of long-term neurological sequeale, including CTE: increasing levels of total or phosphorylated tau protein on sequential samples (hours to days); chronic elevation of total or phosphorylated tau protein on sequential samples (weeks to years); and/or increasing ratio of total to phosphorylated tau protein over any time period (hours to years).

In addition to Tau, other biomarkers have prognostic value. For example, the method further comprises detecting an αB-Crystallin, which is secreted by astrocytes, or a fragment thereof; a Chemokine (C-C motif) ligand 2 or a fragment thereof; an Ubiquitin C-terminal hydrolase (UCH-L1) or a fragment thereof; or a Glial Fibrillary Acidic Protein (GFAP) or a fragment thereof. The markers described herein are measured and computed with Tau values or are measured and computed alone, i.e., without Tau values, as a means to determine whether an individual is likely to develop long-term neurological or neurobehavioral sequelae, including CTE and variant disorders (chronic traumatic encephalopathy with motor neuron disease, chronic traumatic encephalopathy with Parkinsonism). For example, a level of UCH-L1 that is greater than >2 SD above normal control value of about 0.15 ng/mL indicates an increased risk of developing CTE. A level of GFAP that is greater than 2 SD above normal control value of about 250 ng/L indicates an increased risk of developing CTE. Alternatively, these markers are evaluated independently and alone provide prognostic value independent of Tau and other biomarkers.

In addition to determination of Tau or Tau fragment concentration and/or the concentration an αB-Crystallin or a fragment thereof; a Chemokine (C-C motif) ligand 2 (CCL2) or a fragment thereof; UCH-L1 or a fragment thereof; or GFAP or a fragment thereof. The methods optionally include detecting S100-β or a fragment thereof; Neuron-Specific Enolase (NSE) or a fragment thereof; Interleukin-8 (IL-8) or a fragment thereof; Interleukin-6 (Interferon, Beta-2); Myelin Basic Protein (MBP) or a fragment thereof; or αII-Spectrin Breakdown Product (αII-SBDP) or a fragment thereof. These adjunctive markers are useful as confirmation of pathology identified by Tau and/or pTau evaluation. For CCL2 (a potent chemoattractant and sole gating molecule that allows entry of peripheral monocytes into brain/retina), normal control levels are ˜50 pg/ml; levels exceeding this concentration, e.g., in the absence of an infection, indicates a poor prognosis/increased risk of long-term neurological or neurobehavioral sequelae. For αB-Crystallin (which is useful as an independent marker for CTE), normal values in plasma are in the range of 0.3-0.5 ng/ml; similarly, levels exceeding this range indicates a poor prognosis/increased risk of long-term neurological or neurobehavioral sequelae such as CTE. Each of the aforementioned proteins, peptides, or fragments in single or multiple combination with tau, phosphorylated tau, alphaB-crystallin, are used for prognostic purposes.

CTE-linked markers include phosphorylated tauopathy, myelinated axonopathy, microvasculopathy, chronic neuroinflammation, or neurodegeneration in the absence of macroscopic tissue damage or hemorrhage. For example, a CTE-linked marker comprises phosphorylated forms of tau protein or tau protein fragments (tau peptides) and/or biomarkers of myelinated axonopathy; microvasculopathy; blood-brain barrier compromise or loss of structural or functional integrity; chronic neuroinflammation and neuroinflammatory mediators, cytokines, and/or peptides; reactive astrocyte and/or microglial products; and or neurodegeneration in the absence of macroscopic tissue damage or hemorrhage. As described above, the CTE-linked marker is evaluated acutely, i.e., shortly after a suspected insult to the brain, or after a matter of days or at least one week after the blast injury, subconcussive injury, or concussive injury. Monitoring of a subjects condition occurs over weeks, months, and years. For example, the CTE-linked marker is evaluated at least one month after the blast injury, subconcussive injury, or concussive injury, and CTE-linked marker is evaluated at least one year after the blast injury, subconcussive injury, or concussive injury. A blast injury comprises an impact injury or exposure to a blast wind. All of the methods described herein are useful to evaluated patients after a variety of acute head injuries such as acute concussive or subconcussive head injury from blast exposure, impact head injury, acceleration or deceleration head trauma, or other type of single or repeated closed-skull neurotrauma.

A variety of methods are useful to detect levels or concentration of biomarkers in bodily fluids. Preferably, the methods of obtaining the fluids is non-invasive or minimally invasive, e.g., venipuncture or finger prick. Detection of biomarkers is accomplished using a variety of standard methods and reagents. For example, Tau or p-Tau is detected by mass spectrometry. Tau or p-Tau as well as the other biomarkers described above are also detected by using an antigen-specific antibody. Markers are detected using Enzyme Linked Immunosorbent Assay (ELISA) or modification thereof. Other methods include evaluation of a by magnetic resonance imaging, diffusion tensor imaging (dti), positron emission tomography, magnetic resonance imaging and related imaging modalities, magnetic resonance spectroscopy, analysis of cerebrospinal fluid, blood plasma or serum or whole blood.

CTE is evaluated alone or in combination with other markers by mass spectrometry, ELISA, or other quantitative protein detection methodology, or by magnetic resonance imaging, diffusion tensor imaging (DTI), positron emission tomography, magnetic resonance spectroscopy, magnetic resonance imaging and related imaging modalities, any other aforementioned methods deployed with or without combination with specific imaging ligands directed at the aforementioned markers or combined with adjunctive techniques including psychometric evaluation, visual field testing, visual field tracking, retinal imaging, eletroretinography, electroencephalography, pupillary light reflex (pupillometry), analysis of cerebrospinal fluid, blood plasma or serum or whole blood, imaging or spectroscopic analysis of the anterior and posterior chambers of the eye and the tissues comprised therein.

Also within the invention is a device for simulating blast-induced neurotrauma injury comprising a gas-driven shock tube and an internal frame inside the shock tube to position a head of a mammal. The device comprises a gas-driven shock tube and an internal frame inside said shock tube to position head of a mammal 0.1-10 m from the exit of the shock tube and 0.1-10 m from the blast origin. The head of the mammal is not immobilized and a sublethal blast shock wave(s) is delivered to the mammal. The head and neck of the mammal are free to allow flexion, extension, and rotation of the cervical spine in all anatomical planes of motion of said mammal. The diameter of the tube comprises 1-100 cm and the length of the tube comprises 0.5-10 m and the internal frame comprises a cradle to position the head of a mouse 0.56 m from the exit of the shock tube and 4.06 m from a blast origin. The cradle permits flexion, extension, and rotation of the head or the cervical spine in all anatomical planes of motion of said mammal. The device is optionally customized for use with a mouse or other rodent. In the latter case, the animal is positioned without immobilization of the head 0.1 m and up to 10 meters from the exit of the shock tube and 0.1 m and up to 10 meters from the blast origin. Sublethal blast shock waves are delivered to the mouse and the head and neck of the mouse are free to allow flexion, extension, and rotation of the cervical spine in the sagittal and horizontal planes of motion, thereby closely replicating a human injury scenario. In an exemplary device suitable for testing a mouse, the diameter of the tube comprises 25 cm and the length of the tube comprises 5.3 m. In a preferred embodiment, the internal frame comprises a cradle to position the head of a mouse 0.56 m from the exit of the shock tube and 4.06 m from a blast origin.

The assay is carried out by magnetic resonance imaging, diffusion tensor imaging (dti), positron emission tomography, magnetic resonance imaging and related imaging modalities, magnetic resonance spectroscopy, analysis of cerebrospinal fluid, blood plasma or serum or whole blood, imaging or spectroscopic analysis of the anterior and posterior chambers of the eye and the tissues comprised therein.

Compounds are purified and/or isolated. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. Purified also defines a degree of sterility that is safe for administration to a human subject, e.g., lacking infectious or toxic agents.

The assays may involve the use of antibodies such as monoclonal antibodies to detect pTaus. The term antibody encompasses not only an intact monoclonal antibody, but also an immunologically-active antibody fragment, e. g., a Fab or (Fab)₂ fragment, an engineered single chain FV molecule, or a chimeric molecule, e.g., an antibody which contains the binding specificity of one antibody, and the remaining portions of another antibody.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Also within the invention is a mechanical device comprising a field-deployable actuable mechanical device to prevent movement or acceleration of the head relative to the neck, torso, or local environment.

Publications, U.S. patents and applications, and all other references including GENBANK or other sequence databases cited herein, are hereby incorporated by reference.

### BRIEF DESCRIPTION OF THE DRAWINGS

FIGS. 1A-X are a series of photographs. CTE neuropathology in postmortem brains from military veterans with blast exposure and/or concussive injury and young athletes with repetitive concussive injury. (A and E) Case 1, phosphorylated tau (CP-13) neuropathology with perivascular neurofibrillary degeneration in the frontal cortex of a 45-year-old male military veteran with a history of single close-range blast exposure 2 years before death and a remote history of concussion. Whole mount section. Scale bar (E), 100 μm. (B and F) Case 2, phosphorylated tau (CP-13) neuropathology with perivascular neurofibrillary degeneration in the frontal cortex of a 34-year-old male military veteran with history of two blast exposures 1 and 6 years before death and without a history of concussion. Whole mount section. Scale bar (F), 100 μm. (C and G) Case 6, phosphorylated tau (CP-13) neuropathology with perivascular neurofibrillary degeneration in the frontal cortex of an 18-year-old male amateur American football player with a history of repetitive concussive injury. Whole mount section. Scale bar (G), 100 μm. (D and H) Case 7, phosphorylated tau (CP-13) neuropathology with perivascular neurofibrillary degeneration in the frontal cortex of a 21-year-old male amateur American football player with a history of repetitive subconcussive injury. Whole mount section. Scale bar (H), 100 μm. (I) Case 1, phosphorylated tau (CP-13) immune-staining in the parietal cortex revealed a string of perivascular foci demonstrating intense immunoreactivity (areas enclosed by hash lines). Whole mount section. (J) Case 1, phosphorylated neurofilament (SMI-34) immunostaining in adjacent parietal cortex section demonstrating colocalization of multifocal axonal swellings and axonal retraction bulbs surrounding small blood vessels (black circles) relative to perivascular tau foci (areas enclosed by hash lines). Whole mount section. (K) Case 1, human leukocyte antigen-DR (HLA-DR) (LN3) immunostaining in adjacent parietal cortex section demonstrating colocalization of microglial clusters (black circles) relative to perivasculartau foci (areas enclosed by hash lines). Whole mount section. (L) Case 1, high-magnification micrograph of phosphorylated tau (CP-13) immunostaining in the parietal cortex demonstrating string of perivascular phosphorylated tau foci. Whole mount section. (M) Case 1, phosphorylated tau (PHF-1, brown) and phosphorylated neurofilament (SMI-34, red) double immunostaining in parietal cortex demonstrating axonal swellings and a retraction bulb (arrow) in continuity with phosphorylated tau neuritic abnormalities. Whole mount section. Scale bar, 100 μm. (N) Case 1, phosphorylated neurofilament (SMI-34) immunostaining showing diffuse axonal degeneration and multifocal irregular axonal swellings in subcortical white matter subjacent to cortical tau pathology. Whole mount section. (O) Case 1, phosphorylated neurofilament (SMI-34) immunostaining demonstrating perivascular axonal pathology and axonal retraction bulbs near a small cortical blood vessel. Whole mount section. (P) Case 1, activated microglia (LN3) immunostaining showing a large microglial nodule in the subcortical white matter subjacent to cortical tau pathology. LN3 immunostaining was not observed in brain areas devoid of tau pathology. Whole-mount section. Scale bar, 100 μm. (Q) Case 2, phosphorylated tau (CP-13) immunostaining showing diffuse neuronal tau pathology (pre-tangles) in the hippocampal CA1 field. Whole mount section. (R) Case 2, phosphorylated tau (CP-13) pathology in temporal cortex. Whole mount section. (S) Case 1, phosphorylated tau (AT8) immunostaining showing diffuse neuronal tau pathology (pre-tangles) in the hippocampal CA1 field. Whole mount section. (T) Case 1, phosphorylation-independent total tau (Tau-46) immunostaining in the frontal cortex. Whole mount section. (U) Case 3, phosphorylated tau (CP-13) immunostained axonal varicosities in the external capsule of a 22-year-old male military veteran with a history of a single close-range IED blast exposure and remote history of concussions. Whole mount section. (V to X) Case 3, SMI-34 immunostained axonal varicosities and retraction bulbs in the thalamic fasiculus and external capsule. Whole mount sections.

FIGS. 2A-G are a series of line graphs. Free-field pressure (FFP) and intracranial pressure (ICP) dynamics and head kinematics during single-blast exposure in a blast neurotrauma mouse model. (A) Measured incident static blast pressure (blue line) and blast impulse (red line) are compared to equivalent explosive blast waveform expected from 5.8 kg of TNT at a standoff distance of 5.5 m (black line) calculated according to software analysis using ConWep (44). The positive phase terminates at 4.8 ms (t₊=4.8 ms; black hash line). Blast characteristics and waveform structure are comparable to a typical IED fabricated from a 120-mm artillery round (4.53 kg of TNT equivalent charge weight). The measured blast waveform and equivalent TNT blast waveform are in close agreement with a leading shock wavefront followed by a smooth decay. Note that ConWep presents an idealized blast resulting from an above-ground spherical charge and does not model negative-phase pressure transients or modulating factors commonly encountered in military blast scenarios. Reflecting surfaces, bounding structures (for example, crew compartments in armored vehicles, rooms within buildings, walled streets, and alleyways), local geometry, device and deployment characteristics (for example, encapsulation, internal reflectors, and open versus buried deployment), ambient environmental conditions, and other factors strongly influence blast pressure amplitude (positive and negative), phase duration, impulse history, waveform structure, and target interactions (30, 84-86). (B and C) ICP waveform and impulse profile in the brain of an intact living mouse (B) and isolated mouse head severed at the cervical spine (C) subjected to the same blast conditions as in (A). Blast waveforms recorded in the brains of living mice (B) and isolated heads (C) were similar in amplitude to each other and to the measured free-field static pressure. Small differences in the ICP signal waveforms were within the expected range given differences in frequency-dependent transducer response characteristics and experimental preparations. (D) Kinetographic representation of projected Cartesian motion of a representative mouse head during blast exposure as determined by high-speed videography acquired at 100,000 frames per second. Cartesian motion of the head was calculated by tracking a reflective paint mark on the snout. Labeled time points identify corresponding time points in (A) and (E) to (G). (E to G) Relative position (E), angular velocity (F), and angular acceleration (G) of the mouse head referenced to the horizontal (blue) and sagittal (red) planes of motion as determined by analysis of high-speed videographic records obtained during blast exposure. Head acceleration was most significant during the positive phase of the blast shock wave.

FIGS. 3A-T are a series of photographs. Single-blast exposure induces CTE-like neuropathology in wild-type C57BL/6 mice. (A to F) Absence of macroscopic tissue damage (contusion, necrosis, hematoma, or hemorrhage) 1 day (A to C) or 2 weeks (D to F) after exposure to a single blast. Experimental blast conditions were compatible with 100% survival and full recovery of gross locomotor function. (G) Normal astrocytic glial fibrillary acidic protein (GFAP) immunoreactivity in a mouse brain 2 weeks after exposure to sham blast. Whole mount sections. (H) Increased astrocytic GFAP immunoreactivity in the ipsilateral cortex (area enclosed by white hash line), bilateral thalamus (white asterisks), and bilateral hypothalamus (black asterisks) 2 weeks after single-blast exposure. Parenchymal atrophy with ventricular dilation was also observed (white arrowhead). Wholemount sections. (I) Background phosphorylated tau (CP-13) immunostaining in superficial layers of the cerebral cortex 2 weeks after exposure to sham blast. (J) Phosphorylated tau (CP-13) immunostaining in superficial layers of the cerebral cortex 2 weeks after exposure to a single blast. Increased accumulation of phosphorylated tau in the brains of blast-exposed mice was confirmed by quantitative immunoblot analysis (FIG. 5). (K and P) Background phosphorylated neurofilament (SMI-31) immunostaining in the hippocampus 2 weeks after exposure to sham blast demonstrating normal-appearing CA1 pyramidal neurons with no detectable axonal pathology. (L and Q) Increased phosphorylated neurofilament (SMI-31) immunostaining in the hippocampus 2 weeks after exposure to single blast demonstrating pyknotic CA1 pyramidal neurons with nuclear smudging and injured axons with beaded, irregular swellings [arrowhead, (Q); enlargement shown in inset]. (M and R) Faint total tau (Tau-46) immunoreactivity in the soma and processes of pyramidal neurons in the hippocampal CA1 field 2 weeks after exposure to sham blast. (N and S) Increased total tau (Tau-46) immunoreactivity in the soma and processes of pyramidal neurons [arrowheads, (S)] in the hippocampal CA1 field 2 weeks after exposure to single blast. Biochemical abnormalities in total tau expression in the brains of blast-exposed mice were confirmed by quantitative immunoblot analysis (FIG. 5). (O) Faint activated microglial [Ricinus communis agglutinin (RCA)] immunoreactivity in the cerebellum 2 weeks after exposure to sham blast. (T) Increased activated microglial RCA immunoreactivity in the cerebellum indicative of brisk microgliosis [arrowheads, (T)] 2 weeks after exposure to single blast.

FIGS. 4A-N are a series of photographs. Single-blast exposure induces hippocampal ultrastructural pathology in wild-type C57BL/6 mice. (A to G) Normal histology and ultrastructure in the hippocampal CA1 field 2 weeks after sham-blast exposure. (A) Toluidine blue-stained semithick section of the hippocampal CA1 field after sham blast. The CA1 field exhibits normal histological structure with a densely compacted layer of intact pyramidal neurons in the stratum pyramidale (pyr) and profuse dendritic profiles (black arrowheads) in the stratum radiatum (rad). (B to G) Electron micrographs of adjacent ultrathin sections demonstrating normal neuronal, axonal, and perivascular ultrastructure in the hippocampal CA1 field 2 weeks after sham-blast exposure. (B) CA1 pyramidal neurons in proximity to a capillary (asterisk) and endothelial cell. Scale bar, 10 μm. (C) Hippocampal CA1 field with normal stratum pyramidale (above white hash line) and stratum radiatum (below white hash line). Numerous dendrites are evident in the stratum radiatum. Scale bar, 10 μm. (D) Axon field in the stratum alveus demonstrating normal neuropil ultrastructure. Scale bar, 500 nm. (E) Capillary (asterisk) with endothelial cell nucleus (e) in a field of myelinated axons demonstrating normal ultrastructure in the stratum alveus. Scale bar, 500 nm (F) Pyramidal neurons with normal ultrastructure in the hippocampal CA1 field. Scale bar, 2 μm. (G) Myelinated axons in transverse section in proximity to a capillary (asterisk) and endothelial cell (e). Scale bar, 500 nm (H to N) Histological and ultrastructural pathology in the hippocampal CA1 field 2 weeks after single-blast exposure. (H) Toluidine blue-stained semithick section of hippocampus. Clusters of chromatolytic and pyknotic neurons (asterisks) are evident throughout the stratum pyramidale (pyr). Note the marked paucity of dendrites in the stratum radiatum (rad). A tortuous axon (white arrowhead) is present at the boundary between the stratum pyramidale and the stratum oriens. (I to N) Electron micro graphs of adjacent ultrathin cryosections demonstrating widespread ultra-structural pathology in the hippocampal CA1 field 2 weeks after single-blast exposure. (I) Hydropic perivascular astrocytic end-feet (aₑ) surround an abnormal capillary (asterisk) and endothelial cell (e). The astrocytic end-feet are grossly distended and edematous. Numerous vacuoles are scattered throughout the pale cytoplasm. The capillary exhibits an abnormal shape and grossly thickened, tortuous basal lamina (white arrow). A pericyte (p) and numerous electron-dense inclusion bodies are also present. Scale bar, 2 μm. (J) Degenerating pyramidal neurons (nₓ) in proximity to a capillary (asterisk), endothelial cell (e), and swollen, hydropic processes of a perivascular astrocyte in the stratum pyramidale. A neighboring pyramidal neuron (n₁) appears normal. Scale bar, 2 μm. An enlarged field of this same region is also shown. (K) Degenerating myelinated nerve fiber (black star) in the stratum alveus. Scale bar, 500 nm (L) Swollen, hydropic perivascular astrocyte end-feet (aₑ) surrounding a dysmorphic capillary (asterisk) in the hippocampal CA1 field. Note the abnormal endothelial cell (e) with irregularly shaped nucleus and nearby perivascular pericyte (p). The capillary basal lamina (white arrow) is grossly thickened. Lipofuscin granules (white star) are present in an adjacent process. Scale bar, 500 nm. A micrographic montage (corresponding high-magnification micrographs) of this same region reveals the soma and communicating processes of this perivascular astrocyte. (M) Degenerating CA1 pyramidal neuron (nₓ) in the stratum pyramidale of the hippocampal CA1 field. The electron-dense cytoplasm and condensed nucleus of this “dark neuron” correspond to the pyknotic neurons observed in toluidine blue-stained semithick sections (FIG. 4H). A neighboring neuron (n₁) appears normal. Scale bar, 2 μm. (N) Presumptive autophagic vacuoles (v₁, v₂) in a perivascular astrocyte in the hippocampal CA1 field. Scale bar, 500 nm.

FIGS. 5A-F are a series of photographs of electrophoretic gels, and FIGS. 5G-J are a series of bar graphs. Single-blast exposure induces increased brain tau protein phosphorylation in wild-type C57BL/6 mice. (A and B) Immunoblots of brain extracts from the left and right hemispheres of mice probed with monoclonal antibody CP-13 directed against phosphorylated tau protein (pS²⁰²/pT²⁰⁵) 2 weeks after exposure to sham blast (lanes 1 to 4) or single blast (lanes 5 to 8). Note the single broad band that migrated with an apparent molecular mass of 53 kD (arrows) in brains from mice in both groups. (C and D) Immunoblots of brain extracts from the left and right hemispheres of mice probed with monoclonal antibody AT270 directed against phosphorylated tau protein (pT¹⁸¹) using the same homogenates as in (A) and (B). (E and F) Immunoblots of brain extracts from the left and right hemispheres of mice probed with monoclonal antibody Tau 5 directed against total tau protein using the same homogenates as in (A) to (D). Unlike the results shown in the preceding panels, Tau 5 immunoblots revealed an apparent blast-related alteration in tau protein isoform distribution. (G) Densitometric quantitation of CP-13 phosphorylated tau protein (pS²⁰²/pT²⁰⁵) immunolabel in brain homogenates from mice exposed to single blast or sham blast 2 weeks before euthanizing. Mean values±SEM in arbitrary densitometric units (a.u.). P<0.005, two-tailed Student's t test. (H) Densitometric quantitation of CP-13 phosphorylated tau protein (pS²⁰²/pT²⁰⁵) immunolabel in brain homogenates as a proportion of total tau protein (Tau 5) in brain homogenates from mice exposed to single blast or sham blast 2 weeks before euthanizing. Mean values±SEM in arbitrary densitometric units. P<0.05, two-tailed Student's t test. (I) Densitometric quantitation of AT270 phosphorylated tau protein (pT¹⁸¹) immunolabel in brain homogenates from mice exposed to single blast or sham blast 2 weeks before euthanizing. Mean values±SEM in arbitrary densitometric units. P<0.001, two-tailed Student's t test. (J) Densitometric quantitation of AT270 phosphorylated tau protein (pT¹⁸¹) immunolabel in brain homogenates as a proportion of total tau protein (Tau 5) in brain homogenates from mice exposed to single blast or sham blast 2 weeks before euthanizing. Mean values±SEM in arbitrary densitometric units. P<0.001, two-tailed Student's t test.

FIG. 6A is a dot plot, and FIGS. B-F are line graphs. Single-blast exposure induces persistent impairments in axonal conduction velocity and LTP of synaptic transmission in wild-type C57BL/6 mice. (A) Conduction velocity measurements of first peak compound action potential delay as a function of distance between recording electrodes in CA1 pyramidal cell axons in the stratum alveus of hippocampal slices from mice exposed to single blast (red circles, n=13) compared to sham blast (black circles, n=11). Mean±SEM for each group. (B) Representative stimulus-evoked compound action potentials at proximal and distal recording sites (solid and hash lines, respectively) in hippocampal slices from mice exposed to single blast (red) and sham blast (black). Arrows indicate peak negativities used to calculate conduction velocity. (C) Time course of LTP at Schaffer collateral-CA1 synapses evoked by TBS in hippocampal slices from mice exposed to single blast (red circles, n=17) compared to sham blast (black circles, n=11). Each point mean±SEM fEPSP slope of n slices. (D) Time course of LTP at Schaffer collateral-CA1 synapses evoked by bath application of the adenylate cyclase stimulant forskolin (50 μM) plus the type II phosphodiesterase inhibitor rolipram (10 μM; bar, FOR+ROL) in hippocampal slices from mice exposed to single blast (red circles, n=27) compared to sham blast (black circles, n=19). Each point mean±SEM fEPSP slope of n slices. (E) Time course of LTP at Schaffer collateral-CA1 synapses evoked by TBS in hippocampal slices from mice 2 weeks (blue squares, n=10) and 4 weeks after exposure to single blast (red circles, n=7) compared to each other and to sham blast (black circles, n=11). Each point mean±SEM fEPSP slope of n slices. (F) Time course of long-lasting potentiation at Schaffer collateral-CA1 synapses evoked by bath application of the adenylate cyclase stimulant forskolin (50 μM) plus the type II phosphodiesterase inhibitor rolipram (10 μM; bar, FOR+ROL) in hippocampal slices from mice 2 weeks (squares, n=12) and 4 weeks after exposure to single blast (red circles, n=15) compared to each other and to sham blast (black circles, n=19). Each point mean±SEM fEPSP slope of n slices.

FIGS. 7A-C and E are a series of bar graphs, FIG. 7D is a line graph, and FIG. 7F is a series of Barnes maze tracks. Single-blast exposure in wild-type C57BL/6 mice induces persistent hippocampal-dependent learning and memory deficits that are prevented by head fixation (immobilization) during blast exposure. (A to C) Open-field testing showed no effect of blast exposure on gross locomotor function, explorative activity, or thigmotaxis as measured by total distance traveled (A), mean velocity (B), and number of central zone entries (C), respectively, in mice exposed to single blast (red bars, single blast, head free, n=10; blue bars, single blast, head fixed, n=10) or sham blast (black bars, sham blast, n=20). (D to F) Barnes maze testing demonstrated significant impairments in hippocampal-dependent spatial learning acquisition measured by decreasing latency to find the escape box across 4 days of training (D) (two-way ANOVA, P=0.020) and long-term memory assessed by escape box location recall assessed 24 hours after the last training session (E) (**P=0.004, Student's t test). Mice exposed to single blast (red squares, single blast, head free, n=10) are compared to pooled sham-blast control mice (circles, sham blast, n=20). Fixation (immobilization) of the head during blast exposure (blue squares, single blast, head fixed, n=10) reversed blast-induced learning and memory deficits. Arrow-head in (E) represents 5% level predicted by chance selection of the escape box from among the 20-hole choices. (F) Representative Barnes maze tracks obtained on trials 1, 8, and 16 for mice exposed to a single blast (bottom row) compared to sham blast (top row).

FIG. 8 is a drawing of two skulls showing head fixation and direction of blast shock wavefront.

FIG. 9 is a table showing a summary of antibodies. *INC, immunohistochemistry; WB, western blot (protein immunoblot)

FIG. 10 is a table showing blast parameters. *Calculated value based on empirically-determined pressure measurements.

FIG. 11 is a table showing shock tube blast compared to explosive blast.
¹ Blast is comparable to a commonly encountered improvised explosive device (IED) constructed of a 120 mm mortar round with blast equivalence of 4.53 kg of TNT. http://www.gwu.edu/˜nsarchiv/IMG/soldiershandbookiraq.pdf).
² Blast equivalency: 4.50 kg Composition C-4 (5.76 kg TNT) at 5.53 meters calculated using ConWep analysis conducted by William C. Moss, Ph.D., Lawrence Livermore National Laboratory, Livermore, Calif. ConWep software is based on Kingery C. and Bulmash G. (1984) Airblast Parameters from TNT Spherical Air Burst and Hemispherical Surface Burst. Technical Report ARBRL-TR-02555, U.S. Army Ballistic Research Laboratory Proving Ground, Aberdeen, Md. U.S. Army Technical Manual TM 5-855-1, Fundamentals of Protective Design for Conventional Weapons, 1986 (http://www.military-info.com/MPHOTO/p021c.htm). See also Hyde D. W., CONWEP 2.1.0.8, Conventional Weapons Effects Program, United States Army Corps of Engineers, Vicksburg, Miss., 2004.

FIG. 12 is a photograph showing phosphorylated tau axonopathy in a single axon from the brain of a 22-year-old male military veteran with exposure to a single improvised explosive device blast and persistent blast-related traumatic brain injury symptoms. Micrographic montage demonstrating a CP13-immunoreactive axon with beaded (black arrows) and lentiform (white arrows) varicosities along a ˜4 cm length in the external capsule. Calibration bar, 50 m.

FIGS. 13A-B are a series of photographs showing absence of CTE neuropathology in a representative postmortem human brain from 21-year-old male control subject without known history of blast exposure or concussive injury. (A) Absence of specific CP-13 immunostaining for phosphorylated tau protein (pS202/pT205) in the dorsolateral prefrontal cortex. Magnification, ×20. (B) Absence of specific AT8 immunostaining for phosphorylated tau protein (pS202/pT205) in the dorsolateral prefrontal cortex. Magnification, ×10. (C) Absence of specific LN3 immunostaining for MHC class II-positive microglia in the subcortical frontal white matter. Magnification, ×10. Sections were counterstained with cresyl violet.

FIG. 14A is a diagram, and FIGS. 14B-C are photographs showing a schematic and geometry of the murine blast neurotrauma shock tube system. (A) Schematic of the purpose-designed shock tube blast neurotrauma system used in this study. Pressurized gas is delivered into the closed system of the pre-burst compression chamber. Abrupt rupture of a mylar membrane diaphragm separating the compression and expansion chambers initiates a blast shock wave front that traverses the long axis of the 4.5 m shock tube at supersonic velocity (Mach 1.26±0.04). (B) Geometry of blast-induced head motion. Anesthetized mice were secured in a thoracic-protective restraint system positioned inside the shock tube exactly 0.56 m from the open exit of the expansion chamber. High-speed videography enabled precise tracking of a single point on the head in the indicated projected planes of motion. The projected path and kinematics of the head during blast exposure was determined from frame-capture images at a capture rate of 100,000 fps. To translate from the recorded projected head rotation path (X, Y), a motion radius (R) was determined using a pivot point between the scapulae and an endpoint at the snout. The rotational angle of the head (θ) was calculated trigonometrically. (C) Murine blast neurotrauma system was developed in collaboration with the Fraunhofer Center for Manufacturing Innovation at Boston University, Brookline, Mass., and operated at the Neurotrauma Laboratory, Boston University School of Medicine, Boston, Mass.

FIGS. 15A-B are line graphs showing the reproducibility of shock tube blast static and reflected pressure. (A) Reproducibility of shock tube blast wave pressure waveforms assessed with pressure transducer positioned in the reflected (face-on) orientation relative to the direction of the oncoming shock wave. (B) Same shock tube blast waves assessed with pressure transducer positioned in the incident static (side-on) orientation. Note that the static component does not capture dynamic pressure associated with particle motion. The signal at 30 ms (arrow) detected in both orientations was identified as a small reflected wave originating outside the shock tube. Peak pressure was determined by linearly extrapolating the decay of the curve to shock arrival time. Note that the initial pressure spike represents an artifact associated with diffraction at the pressure transducer. In the case shown, the peak static overpressure was 80 kPag with a diffraction artifact spike ˜120 kPag. Pressure data was processed with 20 kHz low-pass filtering.

FIG. 16 is a line graph showing peak reflected and static incident pressure as a function of shock tube burst pressure. Reflected (face-on) and static incident (side-on) pressure demonstrate linear proportionality (.i.e., peak pressure as a function of rupture pressure) over ranges relevant to human blast neurotrauma.

FIG. 17 is a line graph showing shock wave velocity (Mach) regression analysis. Arrival time of the shock wave as a function of the position of the static (side-on) free-field pressure transducer in the shock tube. The pressure transducer was flush mounted inside the shock tube. The slope of the linear regression was 2.32 μs/mm (R²=1.00). The corresponding shock wave velocity yielded a calculated Mach number of 1.26.

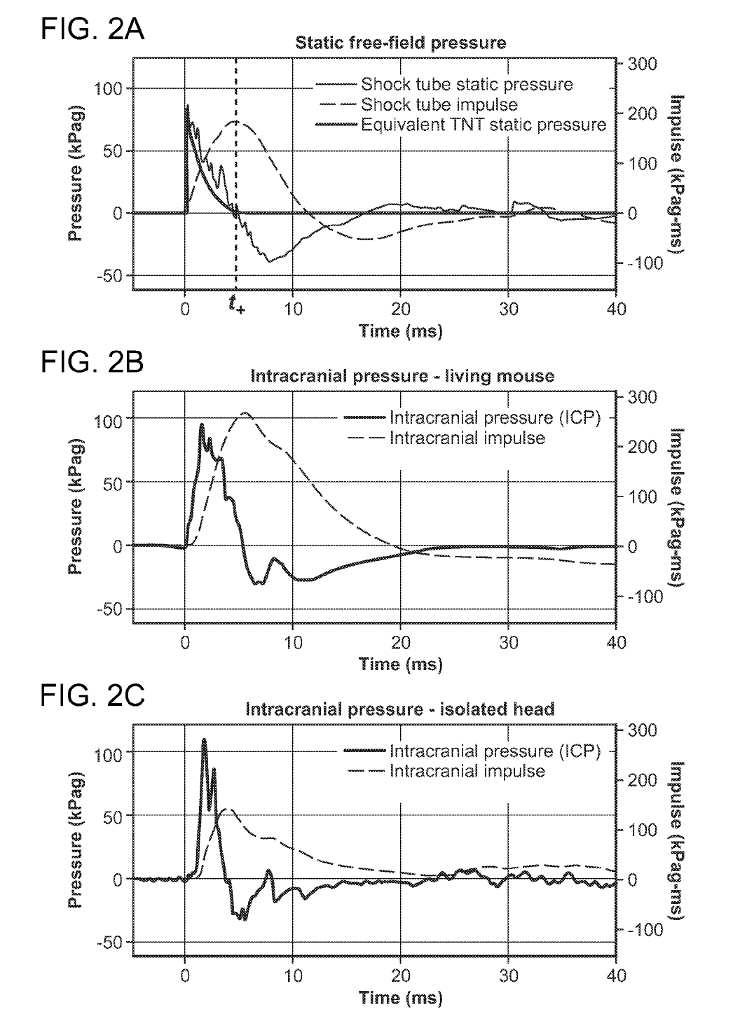

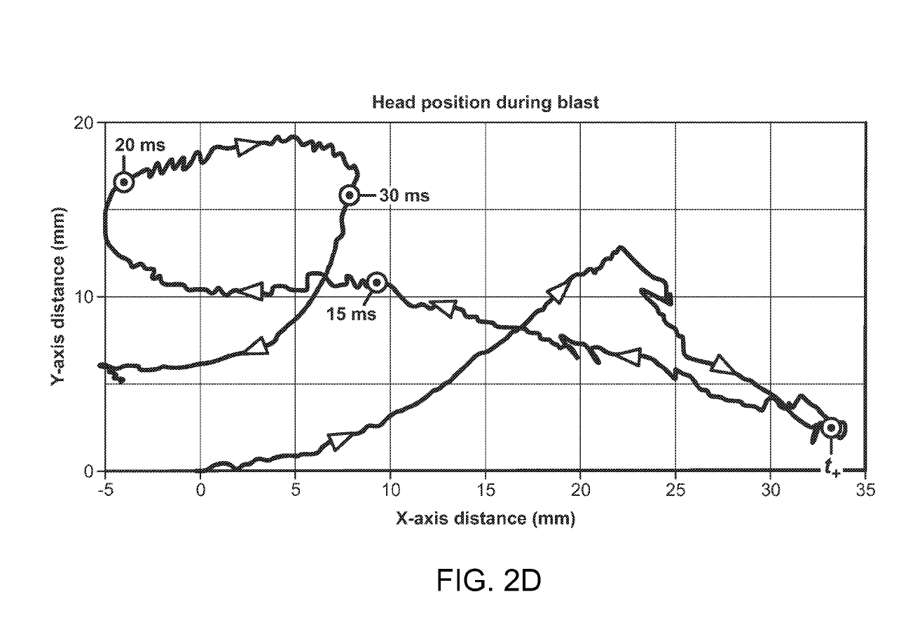

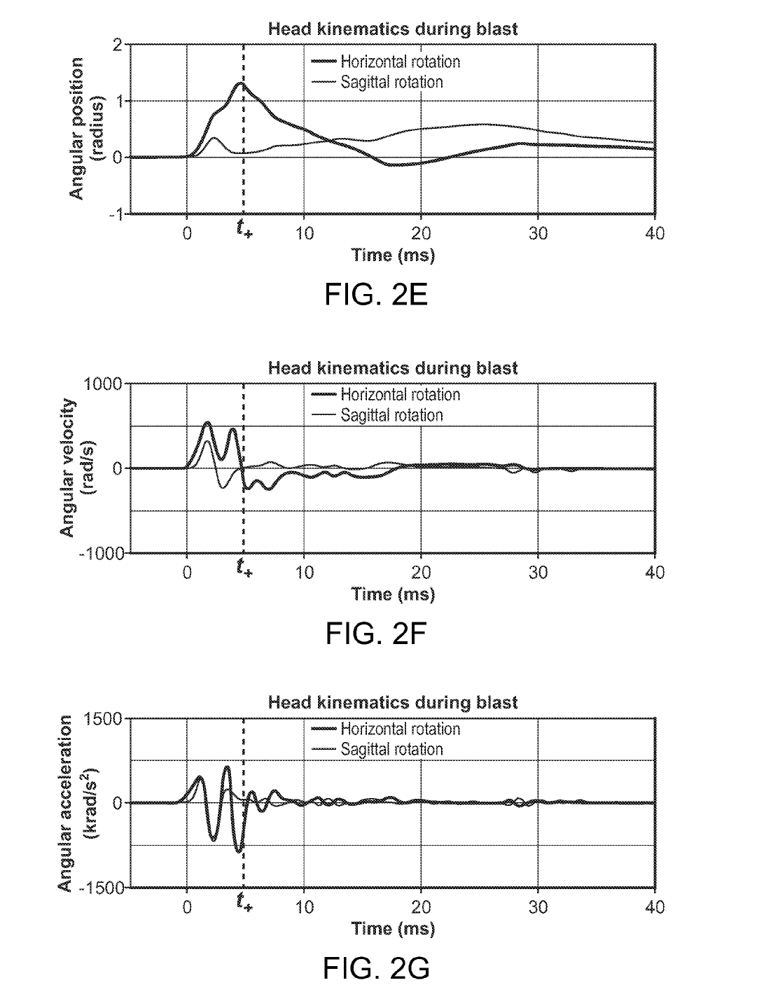

FIG. 18 is a line graph showing X-T wave diagram demonstrating positional and temporal features of the blast shock wave. Blast shock wave front (blue line), shock wave tail (red line), and release wave corresponding to the trailing edge of the compression phase (green line) were calculated according to gas dynamic equations (Liepman & Roshko, Elements of Gas Dynamics, Wiley & Sons, New York, 1957). Interactions between counter-propagating waves in the compression section have been ignored. Wave transmission is shown from the blast origin (x=0) at the interface between the compression and expansion chambers of the shock tube. Mice were positioned 0.56 m from the open exit of the shock tube. Note that near the exit of the shock tube, the release wave has almost caught up with the shock wavefront in agreement with measured waveform at a distance of 4.06 m. The predicted waveform is based on theoretical considerations and the timing of the shock wave at 4.06 m. These data are in good agreement with the amplitude, duration, impulse, and shape of the blast waveform measured experimentally (FIG. 2).

FIGS. 19A-C are a series of photographs showing unperfused C57BL/6 mouse brain 2 weeks after single shock tube blast exposure. Representative unperfused brain from adult male wildtype C57BL/6 mice sacrificed two weeks after exposure to a single shock tube blast did not exhibit gross brain pathology, contusion, necrosis, hematoma, petechial hemorrhage, or focal tissue damage. Dorsal (A), ventral (B), and lateral (C) surfaces of a representative freshly dissected unperfused brain.

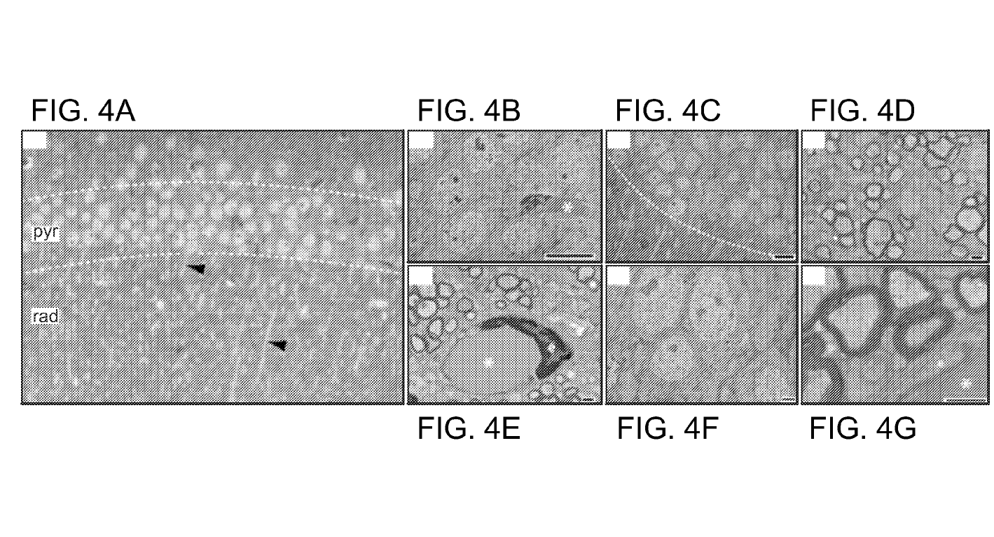

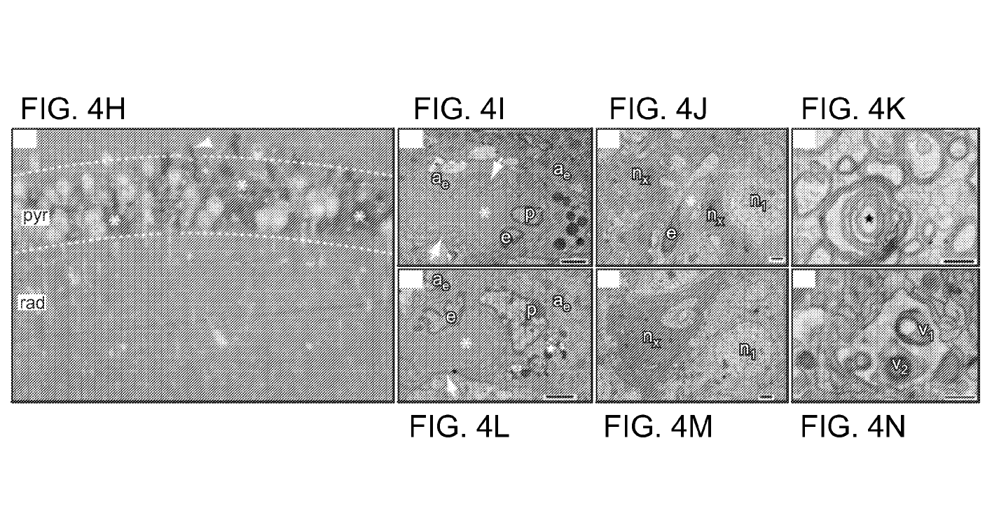

FIGS. 20A-C are photographs showing neuropathology in the CA3 field and dentate gyms in a C57BL/6 mouse brain 2 weeks after exposure to a single shock tube blast. (A) Semi-thick sections of the hippocampus in a C57BL/6 mouse brain two weeks after control exposure to sham blast. Normal histological structure in the hippocampal CA1 and CA3 fields and dentate gyms. (B, C) Toluidine blue-stained semi-thick section of the hippocampus and dentate gyms in a C57BL/6 mouse brain two weeks after exposure to a single shock tube blast. In addition to neuropathology in the CA1 field (FIG. 4), the CA3 field and dentate gyms also demonstrate evidence of extensive neuronal damage, including local neuronal pyknosis (black arrows, B, C), chromatolysis (white arrows, B, C) and dropout (asterisk, C).

FIGS. 21A-H are a series of photographs showing decreased choline acetyltransferase (ChAT) immunoreactivity in the brainstem and neuronal dropout in the cerebellum of C57BL/6 mice weeks after exposure to a single shock tube blast. (A, B) Luxol fast blue/hematoxylin and eosin staining shows cervical spinal cords well-populated with intact motor neurons (arrows) in mice exposed to sham blast (A) or single blast (B). (C, E) Immunohistochemical staining for ChAT in sham blast mice shows robust staining of motor neurons of the cervical spinal cord (C) as well as motor neurons in the nucleus of cranial nerve XII (E). (D, F) In contrast, ChAT immunostaining is markedly decreased in cervical spinal cord (D) and CN XII motor neurons (F) two weeks after single blast exposure. (G) Bielschowsky silver stain reveals intact cerebellar Purkinje cells (arrows, inset) associated with basket cell axons in sham blast mice. (H) Focal loss of cerebellar Purkinje cells and presence of empty baskets (asterisk, inset) in blast-exposed mice. Bar, 100 μm.

FIG. 22 is an electron micrographic montage of the hippocampus CA1 field in a C57BL/6 mouse brain 2 weeks after exposure to a single shock tube blast. EM montage of the CA1 field stratum radiatum shows an enlarged field of the same perivascular profile presented in FIG. 4L. A pale, hydropic astrocyte (A), astrocytic process (Ap), and pathologically swollen astrocytic end-feet (Af) in the vicinity of an irregularly shaped capillary (cap) with a thickened, tortuous basal lamina (black arrows). An endothelial cell (E) with an abnormally contoured multilobed nucleus is located near a perivascular pericyte (P). A process containing lipofuscin granules (lf) is also evident. Bar, 2 μm.

FIGS. 23A-C are high-magnification electron micrographs of the hippocampus CA1 field in a C57BL/6 mouse brain 2 weeks after single blast exposure. These EM micrographs show selected enlarged fields of the same hydropic perivascular profile presented in FIG. 4L. (A) Hydropic perivascular region of the CA1 field demonstrating an edematous astrocytic process (Ap) surrounding an irregularly shaped capillary (cap). An abnormal endothelial cell (E) with a multilobed nucleus is located near a pericyte (P). Lipofuscin granules (lf) are also evident. Black box corresponds to high-magnification micrograph in (B). White box corresponds to high-magnification micrograph in (C). Bar, 2 μm. (B) High-magnification EM micrograph showing lipofuscin granules (lf) and degenerating mitochondria (numbered 1 to 5). A capillary (cap) with a grossly thickened, tortuous basal lamina (black arrows) and adjacent pericyte (P) are also evident. Bar, 500 nm (C) High-magnification EM micrograph showing a perivascular astrocytic process (Ap), abnormal mitochondria (numbers 1-6), and lipofuscin granules (lf). A grossly thickened basal lamina (black arrow) is also evident. Bar, 500 nm.

FIG. 24 is a photograph showing perivascular ultrastructural pathology in the hippocampus CA1 stratum radiatum in a C57BL/6 mouse brain 2 weeks after exposure to a single shock tube blast. Perivascular astrocyte (A) with edematous end-feet (Af) containing numerous dilated vacuoles (vac). Note the endothelial cell (E) with an irregularly contoured nucleus and grossly thickened basal lamina (black arrows). The capillary lumen is not patent (“string vessel”). Bar, 2 μm.

FIG. 25 is a photograph showing perivascular ultrastructural pathology in the hippocampus CA1 stratum radiatum in a C57BL/6 mouse brain 2 weeks after exposure to a single shock tube blast. A swollen astrocytic end-foot (Af) surrounds an endothelial cell (E) with an irregularly contoured nucleus and adjacent pericyte (P). A thickened basal lamina (arrows) and electron-dense inclusion granule (i) are also evident. Dysmorphic myelinated axons (asterisks) are present in the surrounding neuropil (asterisks). Bar, 2 μm.

FIG. 26 is a photographs showing perivascular ultrastructural pathology in the hippocampus CA1 stratum radiatum in a C57BL/6 mouse brain 2 weeks after exposure to a single shock tube blast. Hydropic astrocytic end-foot (Af) containing numerous vacuoles (vac) and a swollen mitochondrion (m) is associated with a thickened, tortuous basal lamina (black arrows) of an adjacent capillary (cap). Two dendritic spines (d), a dystrophic myelinated axon (white asterisk), and a tight junction (white arrowhead) are also evident in this micrograph. Bar, 500 nm.

FIG. 27 is a photograph showing perivascular ultrastructural pathology in the hippocampus CA1 stratum radiatum in a C57BL/6 mouse brain 2 weeks after exposure to a single shock tube blast. Hydropic astrocytic end-feet (Af) surrounding a pericyte (P), endothelial cell (E), and thickened capillary basal lamina (white arrow). Note that the capillary lumen is not patent, an ultrastructural feature that corresponds to string vessels observable by conventional light microscopy. A dystrophic myelinated axon (asterisk) is also evident. Bar, 2 μm.

FIG. 28 is a photograph showing myelin figure in the hippocampus CA1 stratum pyramidale in a C57BL/6 mouse brain 2 weeks after exposure to a single shock tube blast. An edematous astrocytic end-foot (Af) with swollen mitochondria (1-5) and a myelin figure (asterisk). Note the abnormally thickened basal lamina (black arrows) of the adjacent capillary (cap). Bar, 500 nm.

FIG. 29 is a photograph showing a microglial cell amidst myelinated axons in the hippocampus CA1 stratum alveus in a C57BL/6 mouse brain 2 weeks after exposure to a single shock tube blast. A microglial cell (M) is present a field of myelinated nerve fibers in the hippocampus of a blast-exposed mouse. Note the electron dense nucleus and dark cytoplasm that are characteristic features of microglial cells. Bar, 500 nm.

FIGS. 30A-C are photographs showing autophagy and mitophagy in the hippocampus CA1 field in a C57BL/6 mouse brain 2 weeks after exposure to a single shock tube blast. (A) Presumptive degenerating myelinated nerve fiber (black asterisk) in an astrocytic process in the hippocampal stratum alveus. Bar, 500 nm. (B) Astrocytic processes with presumptive multilammelar body (black asterisk), an autophagosomic vesicle variant. Numerous degenerating mitochondria are also evident in this profile (1-6). Bar, 500 nm. (C) Perivascular astrocyte in the stratum pyramidale exhibiting a hydropic process (Ap) with numerous vacuoles (vac) and swollen mitochondria (1, 2). Note the lumen of a nearby capillary (cap). Bar, 500 nm.

FIGS. 31A-B are photographs showing degenerating (“dark”) pyramidal neurons in the hippocampus CA1 stratum pyramidale in a C57BL/6 mouse brain 2 weeks after exposure to a single shock tube blast. (A) “Dark” neurons (N₁, N₂) and adjacent capillary (cap) and endothelial cell (E) in a blast-exposed mouse hippocampus (FIG. 4J). Black box outlines enlarged region in (B) below. (B) Degenerating neurons (N₁, N₂) with electron-dense (“dark”) cytoplasm and irregularly shaped nuclear envelopes (white arrows). A nearby capillary (cap) and endothelial cell (E) are surrounded by grossly swollen astrocytic end-feet (Af) containing dilated vacuoles (vac). A normal-appearing neuron (N3) is present in this micrograph. Bar, 2 μm.

FIG. 32 is a photograpsh showing degenerating (dark) pyramidal neurons in the hippocampus CA1 stratum pyramidale in a C57BL/6 mouse brain 2 weeks after exposure to a single shock tube blast. Degenerating pyramidal neuron (Nx) is characteristically electron-dense (“dark”) and exhibits a convoluted nuclear envelope (white arrows). Vacuoles (vac) and degenerating mitochondria (numbers 1-4) are also present. An adjacent hydropic astrocytic process (Ap) is also evident in this micrograph. Bar, 500 nm.

FIG. 33 is a photograph showing degenerating (dark) pyramidal neurons in the hippocampus CA1 stratum pyramidale in a C57BL/6 mouse brain 2 weeks after exposure to a single shock tube blast. A degenerating pyramidal neuron (Nx) exhibits electron-dense (“dark”) cytoplasm and comparably electron-dense nucleus with an irregularly contoured nuclear envelope (white arrows). “Dark” neurons correspond to the pyknotic pyramidal neurons observed in adjacent toluidine blue-stained semi-thick section (FIG. 4H). Two neighboring pyramidal neurons (N₁, N₂) demonstrate relatively normal ultrastructure. Bar, 2 pm.

FIGS. 34A-B are diagrams showing electrode placements for axonal conduction velocity and synaptic plasticity experiments. (A) Schematic of the hippocampal slice preparation illustrating electrophysiological arrangement for evaluating axonal conduction velocity in the stratum alveus, the hippocampal CA1 axonal output pathway. The positioning of a stimulating electrode and two recording electrodes in stratum alveus of field CA1 are shown relative to local Schaffer collateral-CA1 synaptic circuitry. Recordings of compound action potentials from CA1 pyramidal neurons were used to calculate axonal conduction velocity in the stratum alveus. The time difference between peak negativities at the two recording sites illustrated by each arrow in the CA1 axonal output pathway and distance between the electrodes was used to calculate conduction velocity. (B) Schematic of the hippocampal slice preparation illustrating positioning of stimulation and recording electrodes in stratum radiatum of field CA1 to record Schaffer collateral-evoked field excitatory postsynaptic potentials (fEPSPs) to measure stimulus-evoked and chemically-evoked cAMP-dependent long-term potentiation (LTP) of Schaffer collateral-CA1 synaptic transmission. See Methods for details.

FIG. 35 is a line graph showing Schaffer collateral-CA1 synaptic input-output relations illustrating the absence of long-term effects of blast exposure on baseline synaptic transmission. Hippocampal slices were prepared from mice exposed to a single blast (◯) compared to control sham-blast () four weeks after experimental exposure. Normalized peak fEPSP slope amplitudes are plotted versus Schaffer collateral stimulus intensity. The curves demonstrate that a given intensity of synaptic stimulation elicited the same magnitude response in hippocampal slices from blast-exposed mice compared to sham-blast controls.

FIGS. 36A-B are line graphs showing blast-induced deficits in cAMP-induced long-term potentiation of synaptic transmission at Schaffer collateral-CA1 synapses are bilateral and persistent. (A) Time course of cyclic AMP-induced LTP evoked by bath application of the adenylate cyclase activator forskolin (50 μM) plus the type II phosphodiesterase inhibitor rolipram (10 μM) (FOR+ROL; solid bar) in hippocampal slices from the right hemisphere from mice exposed to a single shock tube blast two weeks (▪, n=6) or four weeks (, n=9) before sacrifice compared to sham-blast control mice (, n=10). (B) Time course of cyclic AMP-induced LTP evoked by bath application of the adenylate cyclase activator forskolin (50 μM) plus the type II phosphodiesterase inhibitor rolipram (10 μM) (FOR+ROL; solid bar) in hippocampal slices from the left hemisphere from mice exposed to a single sublethal blast two weeks (▪, n=6) or four weeks (, n=6) before sacrifice compared to sham-blast control mice (, n=9). Each fEPSP point=mean±S.E.M.

FIG. 37A is a line graph, and FIG. 37B is a table showing an animal model of blast- and concussion-related TBI and sequelae, including CTE.

FIG. 38 is a series of line graphs showing mouse head kinematics during exposure to a single shock tube blast. Single frame from high-speed videographic kinetograph shows the parametric plot of nose position (top left) during blast exposure as a function of time. Nose position was measured in two directions in which the x-axis is parallel to the axis of the shock tube and the y-axis is perpendicular to ground. High-speed videographic record of the blast pressure waveform (bottom left) shows a plot of the coincident free-field pressure dynamics as a function of time. On the right, the radial kinematics, position, velocity and acceleration of blast-induced head movement in both the horizontal (blue) and sagittal (red) planes are shown as a function of time. Static pressure data was processed with 2 kHz low-pass filtering. Angular position data was processed with 500 Hz low-pass filtering.

FIG. 39 is a diagram showing the gene, primary transcript, and isoforms of human brain tau. Human tau gene contains 16 exons with exon-1 as a part of the promoter (upper panel). The human tau primary transcript contains 13 exons, because exons 4A, 6, and 8 are not transcribed in human brain (middle panel). Exons 1, 4, 5, 7, 9, 12, and 13 are constitutive, but exons 2, 3, and 10 are alternatively spliced. The alternative splicing gives rise to six different mRNAs, which are translated to six isoforms (lower panel). These isoforms differ by the absence or presence of one or two 29 amino acids inserts encoded by exon 2 (yellow box) and 3 (green box) in the N-terminal part with either three (R1, R3, and R4) or four (R1-R4) microtubule-binding repeats (black boxes) in the C-terminal part.

FIGS. 40A-B are diagrams and FIG. 40 C is a table showing six isoforms of human CNS tau and phosphorylation sites of Tau. (A) Illustration of the six isoforms of human CNS tau, exons 2, 3, and 10 are alternatively-spliced. Exons 2 and 3 (E2 and E3) encode two different inserts of 28 amino acids near the N-terminus of tau. Absence of E2 and E3 gives rise to 0N tau isoforms, whereas inclusion of E2 produces 1N and inclusion of both E2 and E3 results in 2N tau isoforms. M1-M4 represent the four imperfect-repeat microtubule binding domains, M2 being encoded by exon 10. Lack of M2 produces 3R tau and inclusion results in 4R tau isoforms. The proline-rich domain (PRD) in the centre of the tau polypeptide is indicated. Alternative-splicing produces tau isoforms ranging in size from 352-441 amino acids. (B) Positioning of phosphorylation sites on tau from human Alzheimer brain. Approximately 45 sites have been identified, and they seem to cluster in the PRD and in the C-terminal region, with few sites evident within the microtubule-binding domain of tau. Six of the phosphorylation sites have been identified only by phospho-specific antibody labelling (indicated in orange); the remaining phosphorylation sites have been identified by direct means (mass spectrometry and/or Edman degradation). (C) Phosphorylation sites directly identified in Alzheimer tau and by candidate pathological protein kinases on human tau in vitro. Single letter amino acid abbreviations indicate the sites of all of the phosphorylatable residues in tau (S, serine; T, threonine; Y, tyrosine). Numbering is based on the sequence of the largest isoform of human CNS tau. An asterisk (*) indicates phosphorylation sites directly identified in tau extracted from Alzheimer brain or after incubation of recombinant human tau with selected candidate protein kinases with pathological involvement in Alzheimer's disease. A fully comprehensive listing of tau phosphorylation, including Alzheimer tau, PSPtau, tau from control adult human and foetal rat brain and phosphorylation of recombinant human tau by these and other serine/threonine and tyrosine kinases, is available at http://cnr.iop.kcl.ac.uk/hangerlab/tautable. Grey boxes indicate sites where phosphorylation occurs at one of two or four closely-spaced residues on tau.

FIGS. 41A-B are photographs and FIGS. 41C-E are line graphs showing blast-induced retinal dysfunction at the histological level (FIGS. 41A-B) and functional level (electroretinography, ERG; waveforms in FIG. 41C; B-wave and A-wave data modeling showing same, FIGS. 41D-E). The data was collected by eletroretinography (ERG).

### DETAILED DESCRIPTION

Blast exposure is a known precipitant of brain injury in animals and humans and has been linked to CTE neuropathology. Despite growing awareness of blast-related TBI, the mechanisms of injury and biological basis underpinning blast neurotrauma and sequelae remain largely unknown and a matter of significant controversy. Given the overlap of clinical signs and symptoms in military personnel with blast-related TBI and athletes with concussion-related CTE, we hypothesized that common biomechanical and pathophysiological determinants may trigger development of CTE neuropathology and sequelae in both trauma settings. We combined clinicopathological correlation analysis and controlled animal modeling studies to test this hypothesis.

Blast exposure is associated with TBI, neuropsychiatric symptoms, and long-term cognitive disability. A series of postmortem brains from U.S. military veterans exposed to blast and/or concussive injury were examined. Evidence of chronic traumatic encephalopathy (CTE), a tau protein-linked neuro-degenerative disease, that was similar to the CTE neuropathology was observed in young amateur American football players and a professional wrestler with histories of concussive injuries. A blast neurotrauma mouse model that recapitulated CTE-linked neuropathology was developed in wild-type C57BL/6 mice. Neuropathology was evident 2 weeks after exposure to a single blast. Blast-exposed mice demonstrated phosphorylated tauopathy, myelinated axonopathy, microvasculopathy, chronic neuroinflammation, and neurodegeneration in the absence of macroscopic tissue damage or hemorrhage. Blast exposure induced persistent hippocampal-dependent learning and memory deficits that persisted for at least 1 month and correlated with impaired axonal conduction and defective activity-dependent long-term potentiation of synaptic transmission. Intracerebral pressure recordings demonstrated that shock waves traversed the mouse brain with minimal change and without thoracic contributions. Kinematic analysis revealed blast-induced head oscillation at accelerations sufficient to cause brain injury. Head immobilization during blast exposure prevented blast-induced learning and memory deficits. The contribution of blast wind to injurious head acceleration may be a primary injury mechanism leading to blast-related TBI and CTE. These results identify common pathogenic determinants leading to CTE in blast-exposed military veterans and head-injured athletes and additionally provide mechanistic evidence linking blast exposure to persistent impairments in neurophysiological function, learning, and memory.

The following materials and methods were used to generate the data described herein.

Human subjects. The brain and spinal cord of 12 human subjects (male military veterans, ages 22 to 45 years, mean 32.3 years, with histories of explosive blast and/or concussive injury 1 to 6 years before death, n=4; male athletes with histories of repetitive concussive injury, including 3 amateur American football players and a professional wrestler, ages 17 to 27 years, mean 20.8 years, n=4; male normal controls, ages 18 to 24 years, mean 20.5 years, without known blast exposure, trauma history, or neurological disease, n=4) were procured through the Boston University Alzheimer's Disease Center and Center for the Study of Traumatic Encephalopathy at Boston University School of Medicine. Blast exposure, trauma history, and neurological status at the time of death were determined through review of medical records and interviews with next of kin. Ethical permission to conduct this investigation was approved by Institutional Review Board at Boston University School of Medicine. The study conforms to institutional regulatory guidelines and principles of human subject protection in the Declaration of Helsinki.

Animal subjects. Adult wild-type C57BL/6 male mice (Charles River Laboratories) were group-housed at the Laboratory Animal Science Center, Boston University School of Medicine. All animal experiments used 2.5-month-old mice with 8 to 10 mice per group. Animal housing and experimental use were conducted in accordance with Association for Assessment and Accreditation of Laboratory Animal Care guidelines, in compliance with the Animal Welfare Act and other federal statutes and regulations relating to animals and experiments involving animals, and adherence to principles in the National Research Council Guide for the Care and Use of Laboratory Animals. All studies were approved by Institutional Animal Care and Use Committees at Boston University School of Medicine and New York Medical College.

Histopathological and electron microscopic analyses. Postmortem human brain and spinal cord were received as fresh tissue and as fixed tissue in formalin after processing by medical examiners. Neuropathological analysis followed established protocols at the Boston University Alzheimer's Disease Center and included comprehensive examination for all neurodegenerative conditions. Paraffin-embedded sections from at least 15 brain regions were stained with Luxol fast blue, hematoxylin and eosin, and Bielschowsky silver stain. Mice were euthanized by CO₂ asphyxiation and transcardially perfused with phosphate-buffered saline (PBS). Whole brains were prefixed in 10% neutral buffered formalin, block-sectioned into 2-mm coronal slabs, postfixed in 4% paraformaldehyde, paraffin-embedded, and serially sectioned at 10 μm. A battery of primary detection antibodies (table S1) was used for immunohistopathological analyses. Ultra-structural studies were conducted on fixed brain specimens embedded in Epon, sectioned at 60 nm, stained with uranyl acetate or lead citrate, and examined with a Tecnai-G2 Spirit BioTWIN electron microscope with an AMT 2K CCD camera.

Murine blast neurotrauma model system. A compressed gas-driven shock tube (FIG. 14) was developed in collaboration with the Fraunhofer Center for Manufacturing Innovation at Boston University (Brookline, Mass.) and installed at the Neurotrauma Laboratory, Boston University School of Medicine. This instrument was used to deliver highly reproducible blast waves (FIG. 2A, FIGS. 14-17, and tables S2 and S3). Adult wild-type C57BL/6 male mice (2.5 months) were anesthetized with ketamine (75 mg/kg, intraperitoneally), xylazine (4.3 mg/kg, intraperitoneally), and buprenorphine (0.2 mg/kg, subcutaneously) and secured in the prone position in a thoracic-protective restraint system inside the shock tube (FIG. 14). The head and neck were free to allow flexion, extension, and rotation of the cervical spine in the horizontal and sagittal planes of motion to model conditions relevant to military blast exposure. Maximum burst pressure compatible with 100% survival and no gross motor abnormalities was ascertained empirically (table S2). Experimental blast parameters (incident static pressure, 77±2 kPag; blast overpressure rise time, 38±3 μs; compressive phase duration, 4.8±0.1 ms; shock wave velocity, 1.26±0.04 Mach; calculated blast wind velocity, 150 m/s=336 miles/hour; table S2) closely approximate explosive blast produced by detonation of 5.8 kg of TNT measured at a standoff distance of 5.5 m [ConWep analysis (Hyde, D. W. CONWEP 2.1.0.8, Conventional Weapons Effects Program, United States Army Corps of Engineers, 2004); table S3]. This blast exposure is within the range of typical IED detonations and standoff distances associated with military blast injury. Anesthetized mice were exposed to a single blast or sham blast, removed from the apparatus, monitored until recovery of gross locomotor function, and then transferred to their home cage.

Static and reflected FFP measurements. Assessment of static and reflected FPP was assessed by two piezoelectric pressure sensors (model HM102A15, PCB Piezotronics) placed in the shock tube at the same axial distance relative to the head of the animal subjects. A static pressure (side-on) sensor was flushed-mounted inside the shock tube. A second transducer was positioned with the detector facing into the shock tube in a reflected pressure (face-on) orientation. Pressure signals were processed with a PCB signal conditioner (model 482C05, PCB Piezotronics) and recorded at a frequency of 5 MHz with a digital oscilloscope (640Zi WaveRunner, LeCroy). Voltages were converted to pressure with calibration data provided by the manufacturer and processed with 2-kHz low-pass filtering.

ICP measurements. ICP measurements were conducted with a broad-bandwidth piezoelectric needle hydrophone (NP10-3, DAPCO Industries) with a 0.6-mm-diameter element sheathed in a stainless steel hypodermic needle. Pressure sensitivity was flat to within ±3 dB for frequencies ranging from 1 Hz to 170 kHz. The needle hydrophone was inserted into the hippocampus at −3.00 mm caudal to the bregma suture, +3.50 mm lateral to the sagittal suture, and +2.00 mm ventral to the skull surface. For ICP measurements, the head was immobilized to prevent displacement of the pressure sensor. Piezoelectric voltage signals were recorded by a digital oscilloscope (640Zi WaveRunner, LeCroy) and converted to pressure units with calibration data supplied by the manufacturer and processed with 20-kHz low-pass filtering. Post-acquisition processing was performed with Matlab 2009 (MathWorks).

High-speed videographic kinematic analysis. High-speed videography was conducted with a FASTCAM SA5 camera (Photron USA Inc.; courtesy of Tech Imaging) operated at 10-ps frame capture rate. Videographic records were reassembled with open-source ImageJ software and processed in Matlab (MathWorks). Angular position and motion of the head were assessed by tracking a reflective paint mark on the snout, calculated by assuming a central pivot point between the scapulae (FIG. 15B), and processed with 500-Hz low-pass filtering (FIG. 2, D to G).

Hippocampal electrophysiology. Mice were decapitated under deep isoflurane anesthesia, and the brains were quickly removed, hemisected, and sectioned with a Leica model VT 1200S vibratome at 350 μm. Slices were fixed to a stage with cyano-acrylate adhesive and immersed in oxygenated artificial cerebrospinal fluid (126 mM NaCl, 3 mM KCl, 1.25 mM NaH₂PO₄, 1.3 mM MgCl₂, 2.5 mM CaCl₂, 26 mM NaHCO₃, 10 mM glucose, saturated with 95% O₂ and 5% CO₂) at 32° C. Experimental drugs were bath applied in the perfusate at a rate of 3 ml/min. Axonal conduction velocity was assessed with a recording electrode placed in CA1 stratum alveus. Schaffer collateral-CA1 synaptic transmission and plasticity were assessed with a recording electrode in the CA1 stratum radiatum.

Hippocampal-dependent learning and memory. Open-field testing (Med-Associates) was used to assess gross loco-motor function, exploratory activity, and thigmotaxis. Hippocampal-dependent learning acquisition and memory retention were evaluated in the Barnes maze (Barnes, C. A., 1979, J. Comp. Physiol. Psychol 93:74-104). Spatial learning was assisted by visual cues in the environment that remained constant across test sessions. Movement was tracked and recorded electronically (Stoelting). Latency to find the escape box, trajectory velocity to the escape box, and total trajectory distance were assessed and recorded daily in four sessions conducted over 4 days. Memory retrieval was electronically assessed by recording the number of nose pokes in blank holes as a percentage of total nose pokes recorded 24 hours after completion of the learning protocol.

Quantitative assessment of phosphorylated and total tau protein. Quantitative immunoblot analysis was conducted with left and right hemisected brains obtained from PBS-perfused mice 2 weeks after exposure to a single blast (n=6 mice) or sham blast (n=6 mice). Snap-frozen hemisected brain specimens were thawed, resuspended in 0.7 ml of protease-phosphatase inhibitor buffer, and homogenized. Protein concentrations were normalized and equal sample volumes were subjected to standard polyacrylamide gel electrophoresis in duplicate. Immunoblot detection used monoclonal antibody AT270 (Innogenetics) directed against tau protein phosphorylated at Thr¹⁸¹ (pT¹⁸¹), monoclonal antibody CP-13 directed against tau protein phosphorylated at Ser²⁰² (pS²⁰²) and Thr²⁰⁵, or monoclonal antibody Tau 5 directed against phosphorylation-independent tau protein. Other phosphorylated residues (FIG. 40B) and/or combinations thereof are detected in a similar manner, and additional antibodies to detect Tau and/or pTau are known in the art, e.g., Augustinack et al., 2002, Acta Neuropathol. 103:26-35. Triplicate densitometry measurements were analyzed with open-source ImageJ software. A commercial ELISA kit was used to quantitate murine-specific tau protein phosphorylated at Ser¹⁹⁹ (Invitrogen). Frozen brain samples were homogenized in eight volumes of 5 M guanidine-HCl and 50 mM tris (pH 8) followed by five passes in a glass Teflon homogenizer. Homogenates were mixed for 3 hours, diluted into PBS containing protease inhibitors, and centrifuged for 20 min at 16,000 g. Supernatants were diluted and assayed in quadruplicate according to the manufacturer's instructions.

Statistical analyses. Comparisons of axonal conduction velocity and LTP magnitude were conducted with repeated-measures multifactorial ANOVA with Bonferroni-Dunn post hoc correction. Longitudinal neurobehavioral data were analyzed by repeated-measures ANOVA. Memory retrieval was evaluated by ANOVA. Statistical significance was preset at P<0.05.

Histopathology. Processing of human brains followed established procedures and protocols at the Boston University Alzheimer's Disease Center, Boston, Mass., and included comprehensive neuropathological analysis of neurodegenerative conditions. Human brain and spinal cord specimens were received as fixed tissue in formalin after processing by medical examiners. Paraffin-embedded sections from at least 15 brain regions were stained with Luxol fast blue, hematoxylin and eosin, and Bielschowsky silver stain. Sections evaluated by immunohistochemistry utilized a battery of primary antibodies (table S1), chromogen visualization (Vectastain Elite ABC Kit, Vector Labs, Burlingame, Calif.), and cresyl violet counterstaining. For histological experiments involving mice, animals were euthanized by CO₂ asphyxiation according to IACUC-approved protocol followed by transcardial gravity perfusion with phosphate-buffered saline (PBS, Sigma-Aldrich, St Louis, Mo.). Brains were rapidly removed from the calvarium and placed in 10% neutral buffered formalin for 2 hours, then transferred to PBS. Coronal slabs (2 mm) were obtained by block sectioning, fixed in 4% paraformaldehyde for 2 hours, embedded in a single paraffin block, and serially sectioned at 10 μm. Sections were processed for immunohistochemistry with a battery of primary antibodies (table S1) and visualized by Vectastain Elite ABC Kit (Vector Labs, Burlingame, Calif.). Slides were developed according to manufacturer's instructions for exactly the same incubation time and counterstained with hematoxylin. For double immunostained sections, tissue was blocked with avidin and biotin before primary antibody incubation and visualized with DAB and aminoethylcarbazole according to manufacturer's instructions (Vector Laboratories, Burlingame, Calif., USA). Bielschowsky silver stain was performed using 20% AgNO₃ titrated with ammonia and developed with HNO₃ and citric acid and unbuffered formalin.

Electron Microscopy. Small pieces (1-2 mm cubes) of harvested brain were fixed in 2.5% glutaraldehyde with 2.5% paraformaldehyde in 0.1M sodium cacodylate buffer (pH 7.4) overnight at room temperature, washed in 0.1M cacodylate buffer, postfixed with 1% osmium tetroxide (OsO₄) with 1.5% potassium ferrocyanide (KFeCN₆) for 1 hour, then washed in water. The specimens were then incubated in 1% aqueous uranyl acetate for 1 hr, washed, and sequentially dehydrated in increasing grades of alcohol (10 min each in 50%, 70%, 90%, 100%, 100%). Samples were treated in propylene oxide for 1 hr and infiltrated overnight in a 1:1 mixture of propylene oxide and TAAB Epon (Marivac Canada Inc., St. Laurent, Canada) and polymerized at 60° C. for 48 hrs. Ultrathin sections (60 nm) were cut on a Reichert Ultracut-S microtome, placed on copper grids, stained with lead citrate or uranyl acetate, and examined using a Tecnai-G2 Spirit BioTWIN electron microscope. Images were acquired with an AMT 2K CCD camera.

Murine Blast Neurotrauma Model. A compressed gas-driven shock tube (25 cm diameter; 5.3 m tube length; FIG. 13) developed in collaboration with the Fraunhofer Center for Manufacturing Innovation at Boston University, Boston, Mass., and installed at the Murine Neurotrauma Laboratory, Boston University School of Medicine, Boston, Mass. was used to deliver highly-reproducible sublethal blast shock waves relevant to human blast injury (FIG. 13-17). Adult wildtype C57BL/6 male mice (Charles River Laboratories, Wilmington, Mass.) at 2.5-months-of-age were anesthetized with ketamine (75 mg/kg, i.p.), xylazine (4.3 mg/kg, i.p.), and buprenorphine (0.2 mg/kg, s.c.), secured in the prone position with a wire mesh holder, and inserted into a custom-fabricated restraint system that protected the thorax. The assembly was then fixed to an internal frame inside the shock tube with the unprotected head positioned exactly 0.56 m from the exit of the shock tube and 4.06 m from the blast origin (FIG. 13). In order to model conditions relevant to human blast exposure conditions, the head and neck were free to allow flexion, extension, and rotation of the cervical spine in the sagittal and horizontal planes of motion. We empirically determined the maximum burst pressure (303±9 kPag) and corresponding blast parameters compatible with 100% survival with no gross motor abnormalities 24 hours following blast exposure (table S2). Anesthetized mice were exposed to a single sublethal shock tube blast (table S2) or sham blast, removed from the apparatus, and monitored until recovery of gross locomotor function and exploratory activity. Mice were then transferred to their home cage.

Blast Comparators. Experimental shock tube blast parameters (i.e., peak static pressure amplitude, duration, and impulse) used in this study closely approximated characteristics of explosive blast produced by detonation of 5.8 kg of 2,4,6-trinitrotoluene (TNT) or 4.5 kg of Composition C-4 explosive measured at a standoff distance of 5.53 m (table S3) analyzed using the Conventional Weapons Effects Program (ConWep). For comparison, an improvised explosive device (IED) commonly encountered by U.S. military personnel utilizes a 120 mm mortar round equivalent to 4.53 kg of TNT (1st Infantry Division Soldier's Handbook to Iraq, U.S. Army, at weblink: http://www.gwu.edu/˜nsarchiv/IMG/soldiershandbookiraq.pdf accessed Jan. 2, 2012. The blast exposure utilized in this study was comparable to experimental conditions in recent studies utilizing a shock tube (Independent Panel on the Safety and Security of United Nations Personnel in Iraq. Available at the following weblink: http://www.un.org/News/dh/iraq/safety-security-un-personnel-iraq.pdf. Accessed Feb. 24, 2012; Warden et al.m 2005, J Neurotrauma 22: 1178) or detonated explosives (Murray et al., 2005, Mil Med 170: 516-520) to model moderate intensity blast exposure relevant to the military.

Static and Reflected Free-Field Pressure Measurements. Assessment of static (side-on) and reflected (face-on) free-field pressure (FFP) during blast exposure was assessed by two piezoelectric pressure sensors (Model HM102A15, PCB Piezotronics Inc., Depew, N.Y., USA) placed in the shock tube at the same axial distance as the head of the mouse. One sensor was flushed-mounted inside the shock tube and secured in a static pressure (side-on) orientation relative to the blast shock wave. The second transducer was positioned with the detector facing into the shock tube in a reflected pressure (face-on) orientation relative to the blast shock wave. With respect to the reflected pressure sensor, the measured pressure magnitude does not capture the total pressure (i.e., stagnation pressure) of the blast wave as a consequence of the small size and geometry of the sensor system relative to the blast wave produced by our shock tube system. However, the reflected pressure transducer was comparable in size to the mouse head and thus recorded relevant pressure incident to the head during blast exposure. Pressure signals in both orientations were processed through a PCB signal conditioner (Model 482C05, PCB Piezotronics Inc., Depew, N.Y., USA) and recorded at a frequency of 2 MHz using a digital oscilloscope (640Zi Waverunner; LeCroy, Chestnut Ridge, N.Y.). Voltages were converted to pressure using calibration data.

Intracranial Pressure Measurements. Intracranial pressure (ICP) measurements were conducted with a broad-bandwidth piezoelectric needle hydrophone (NP10-3; DAPCO Industries Inc., Oak Creek, Wis.) with a 0.6 mm diameter active element sheathed in a standard #19 gauge hypodermic needle (length, 75 mm; o.d., 1 mm). Pressure transducer sensitivity was flat to within ±3 dB for frequencies ranging from 1 Hz to 170 kHz. The needle hydrophone was inserted into the hippocampus (−3.00 mm caudal to the bregma suture, +3.50 mm lateral to the sagittal suture, +2.00 mm ventral to skull surface according to the atlas of Franklin and Paxinos The Mouse Brain in Stereotaxic Coordinates, 3rd Ed., Elsevier Academic Press, Boston, 2008. For ICP measurements, the head was secured in place to prevent intracranial displacement during blast exposure. ICP piezoelectric voltage signals were recorded by a digital oscilloscope (640Zi Waverunner; LeCroy, Chestnut Ridge, N.Y.) converted to pressure using calibration data derived from substitution experiments with calibrated transducers over a frequency range up to 2 MHz. Post-acquisition processing was performed with Matlab 2009 software (MathWorks, Natick, Mass., USA).

High-Speed Videography and Kinematic Analysis. High-speed videography was conducted with a FASTCAM SA5 camera and software (Photron USA Inc., San Diego, Calif.) operated at a 10 μs frame capture rate (100 kHz). Initial post-acquisition analysis of individual frames was conducted using ImageJ software (NIH, Bethesda, Md.). All subsequent processing was carried out in Matlab (MathWorks, Natick, Mass.). Angular rotation of the head was calculated by assuming a central pivot point between the scapulae. Cartesian motion of the head was calculated by tracking a paintmarked nose spot.

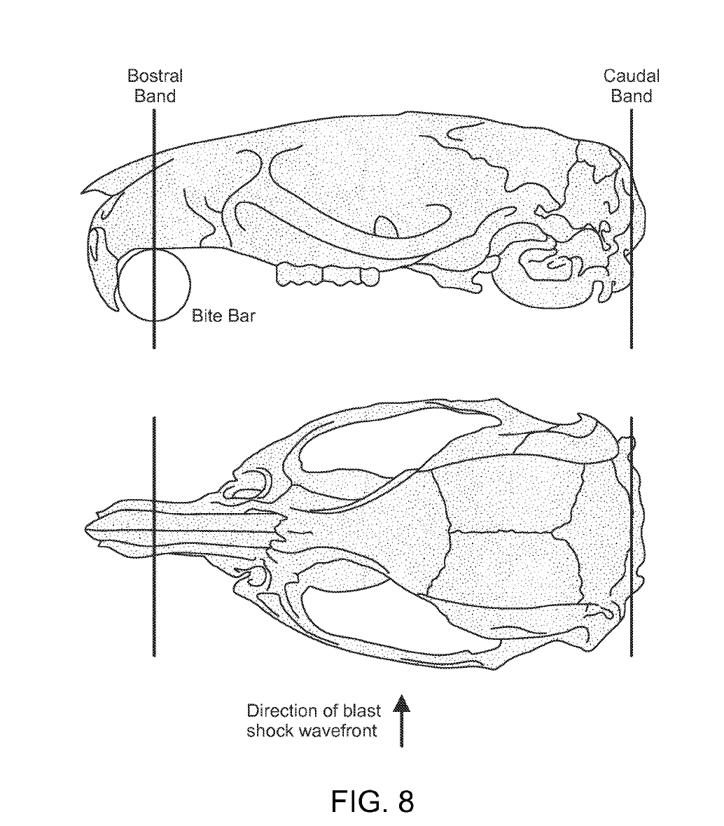

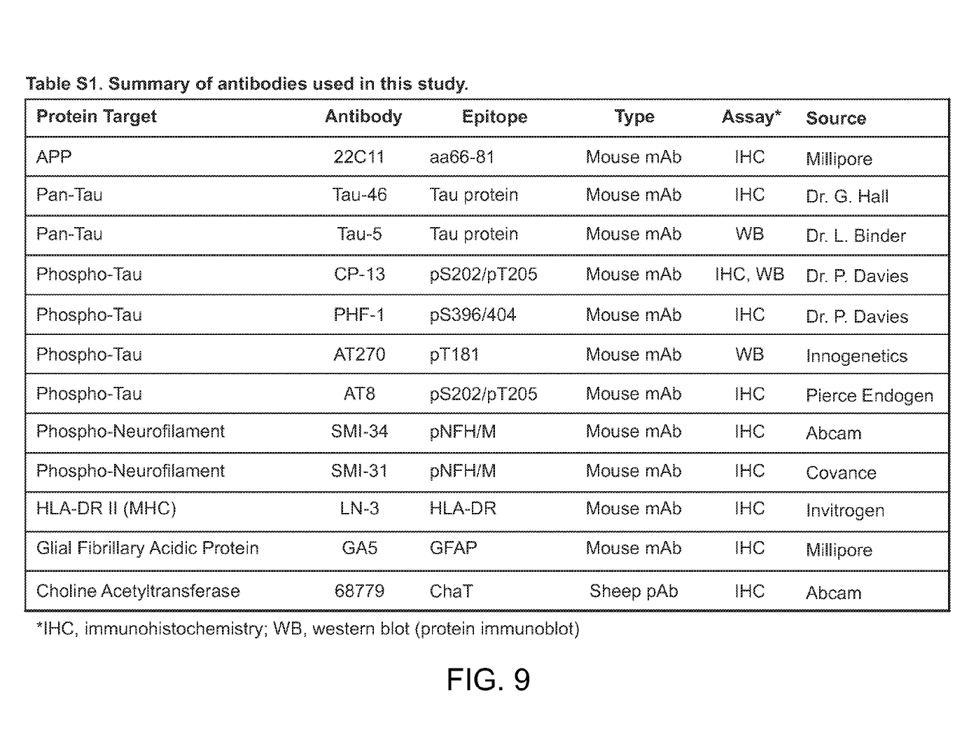

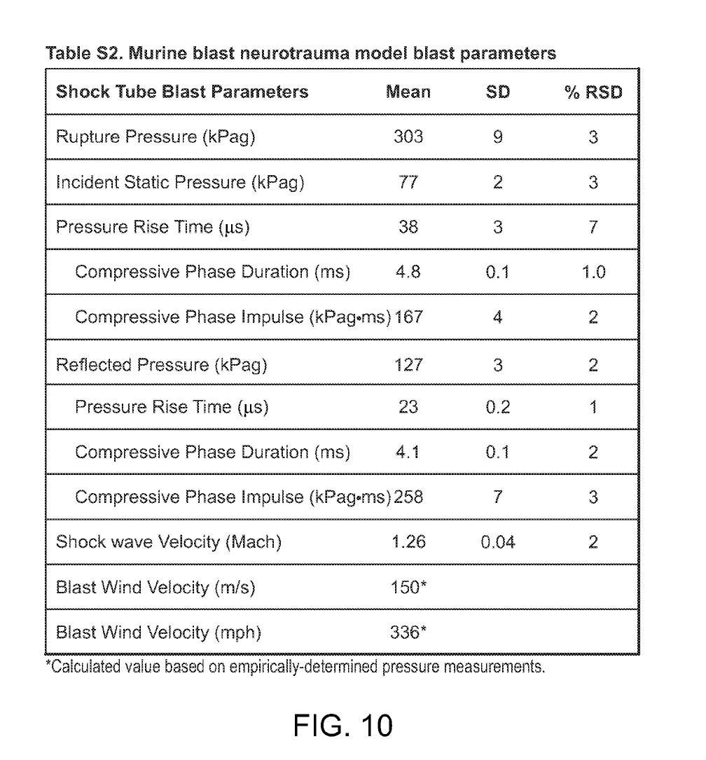

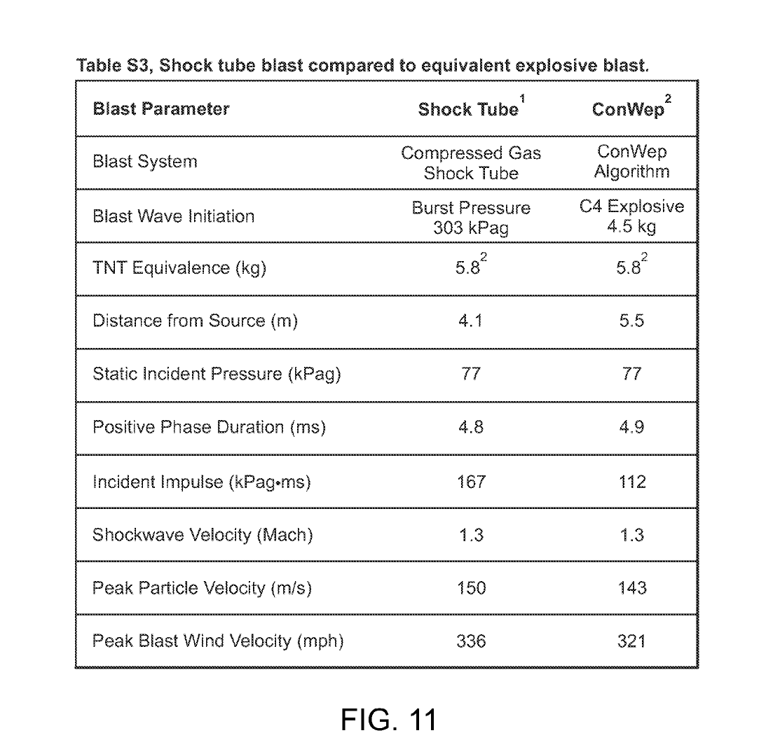

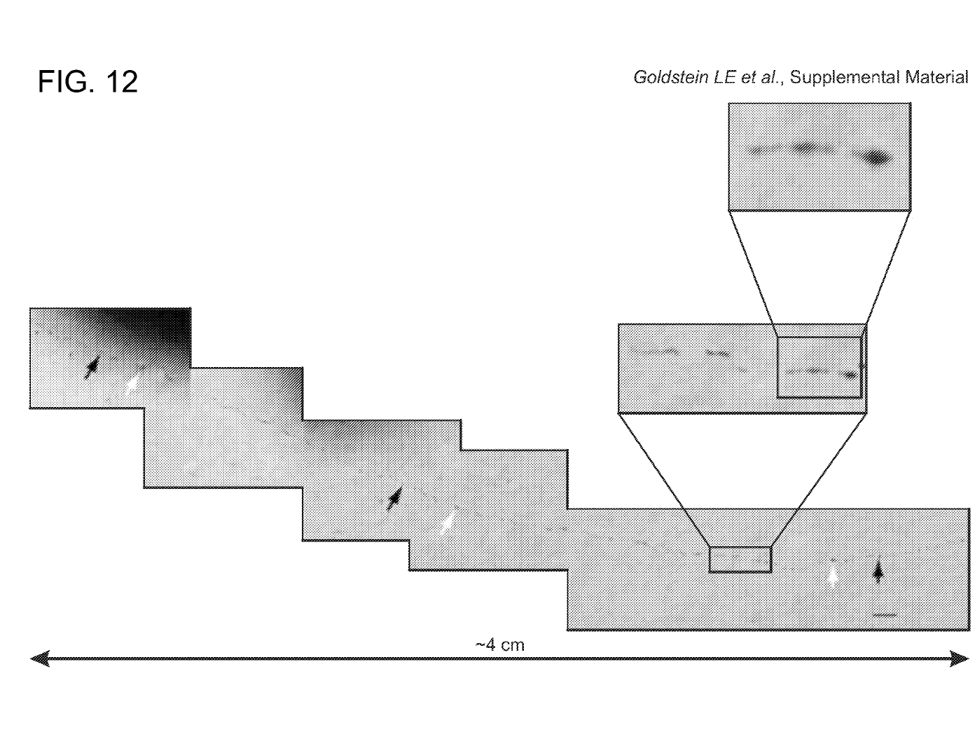

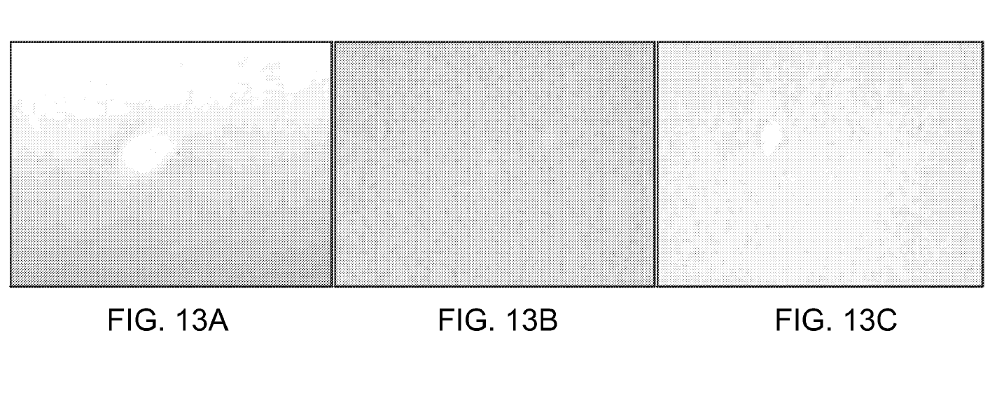

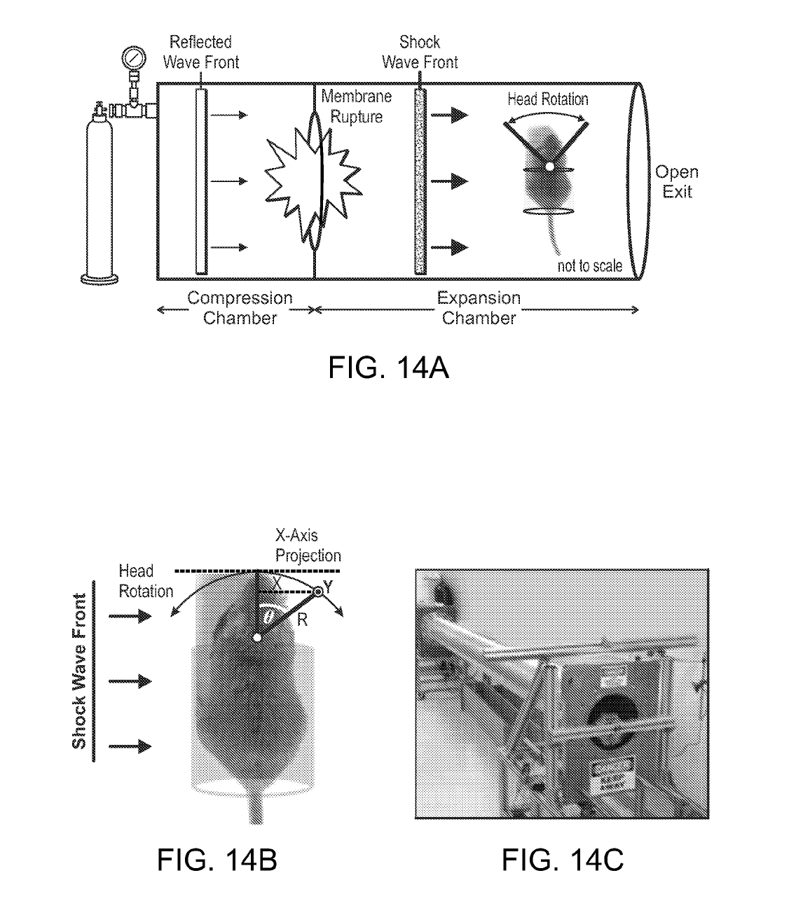

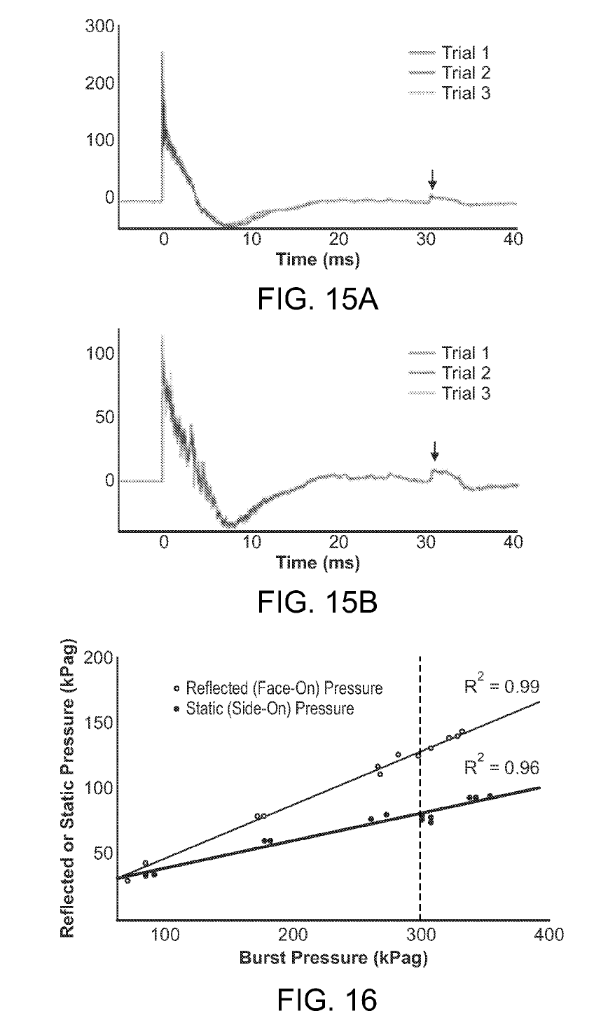

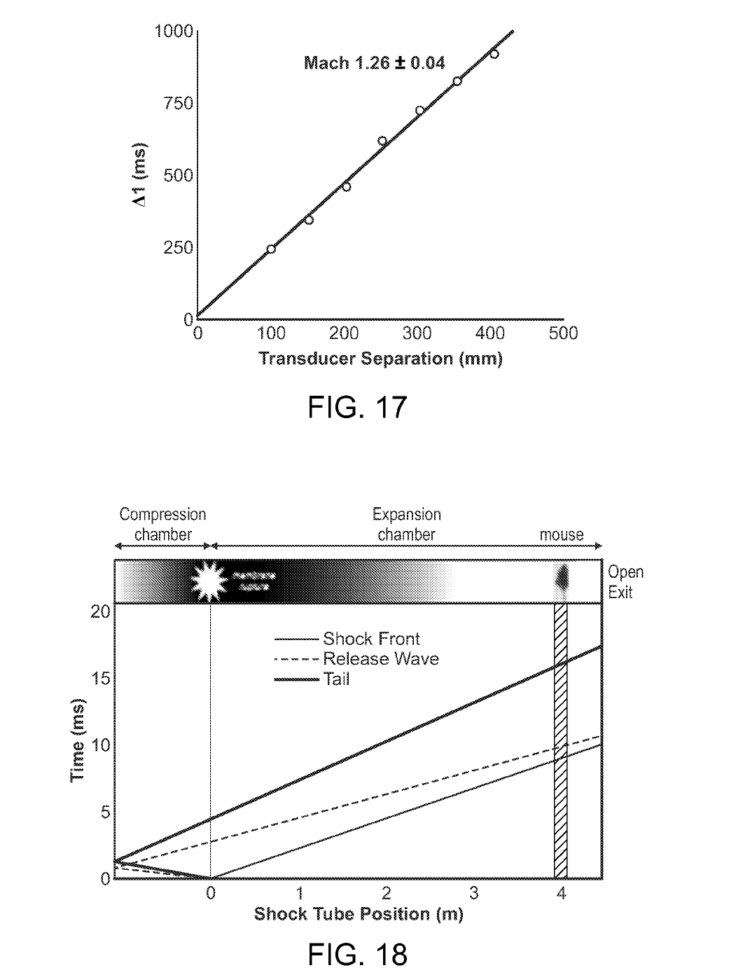

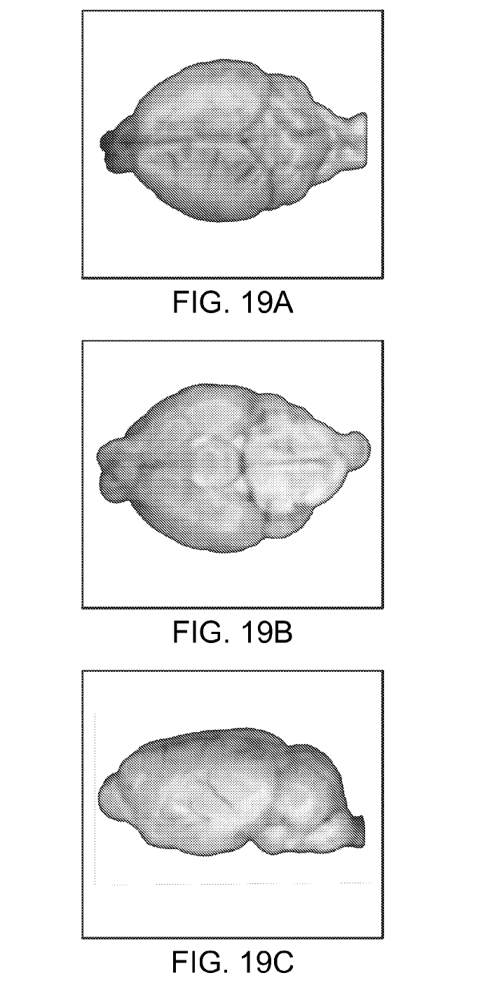

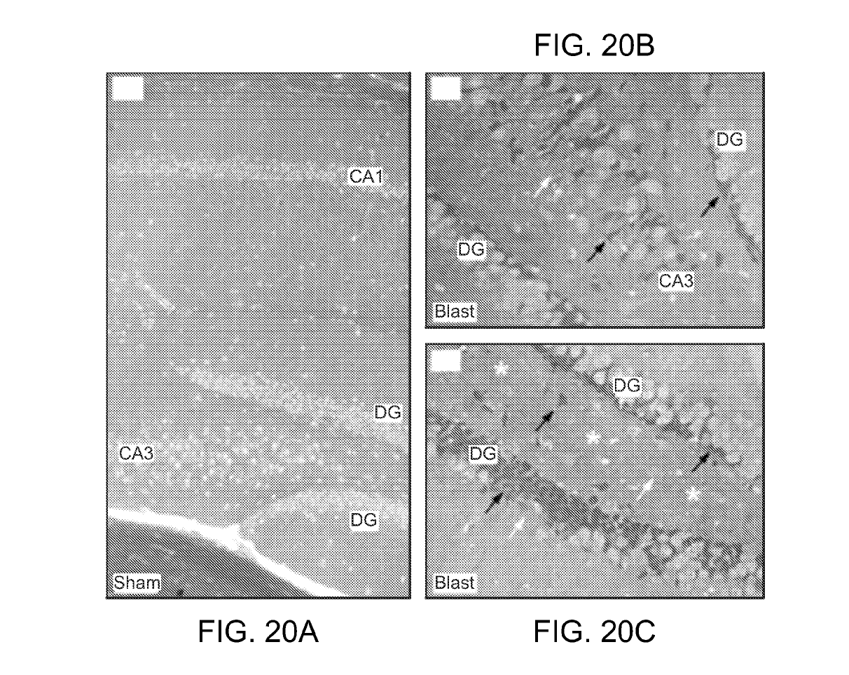

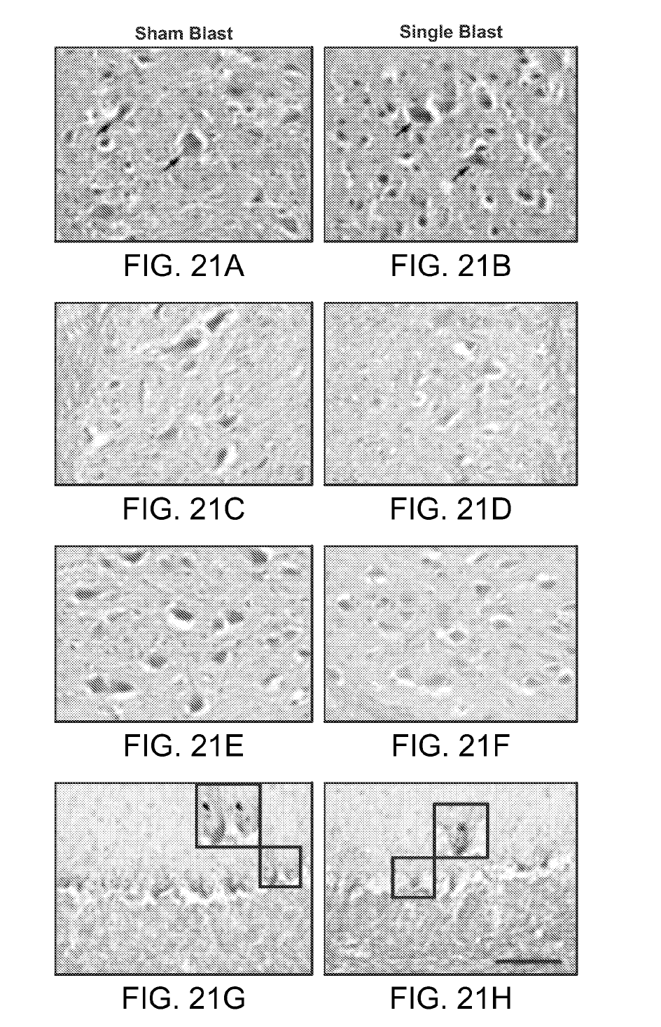

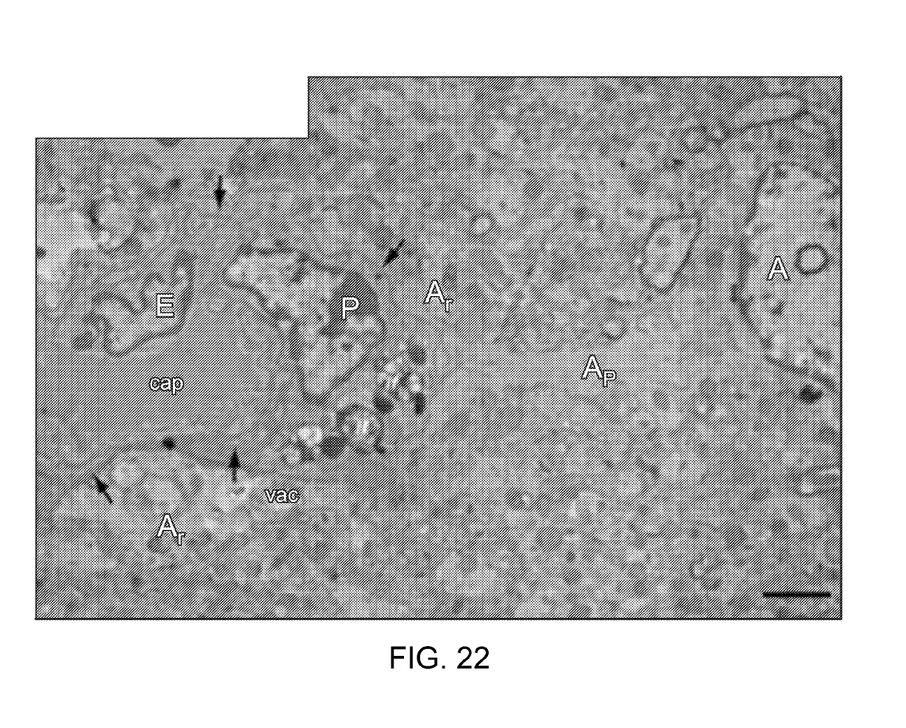

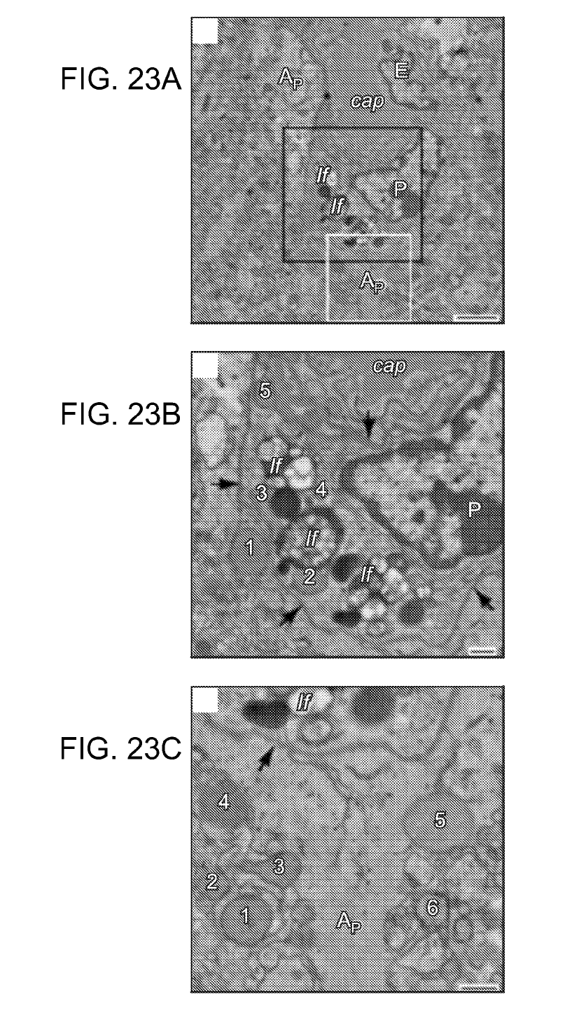

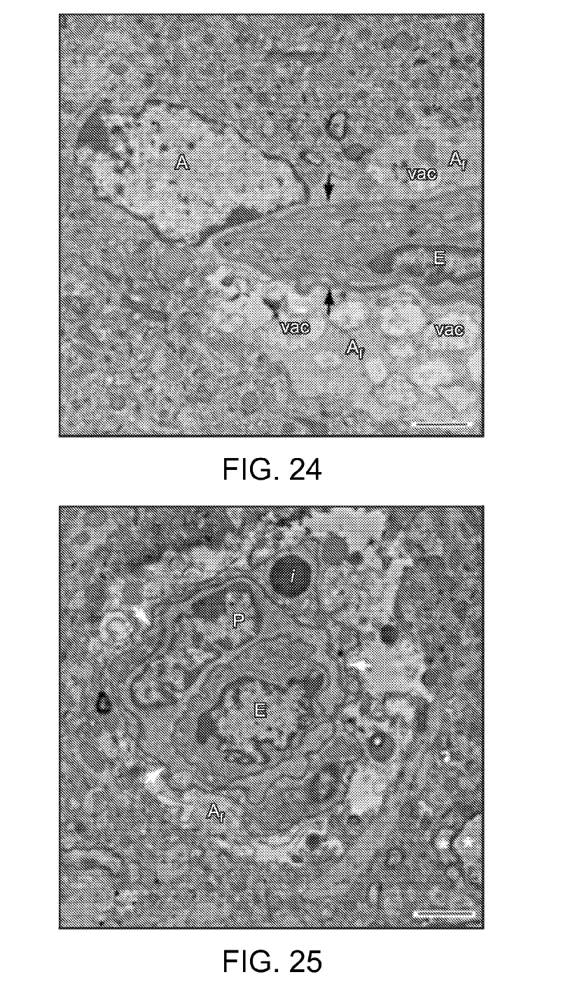

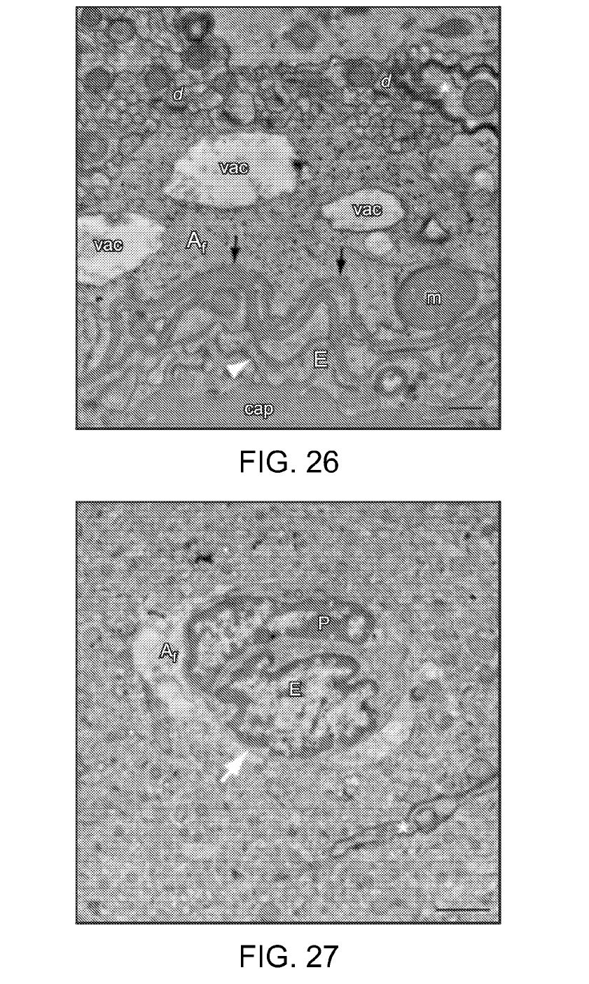

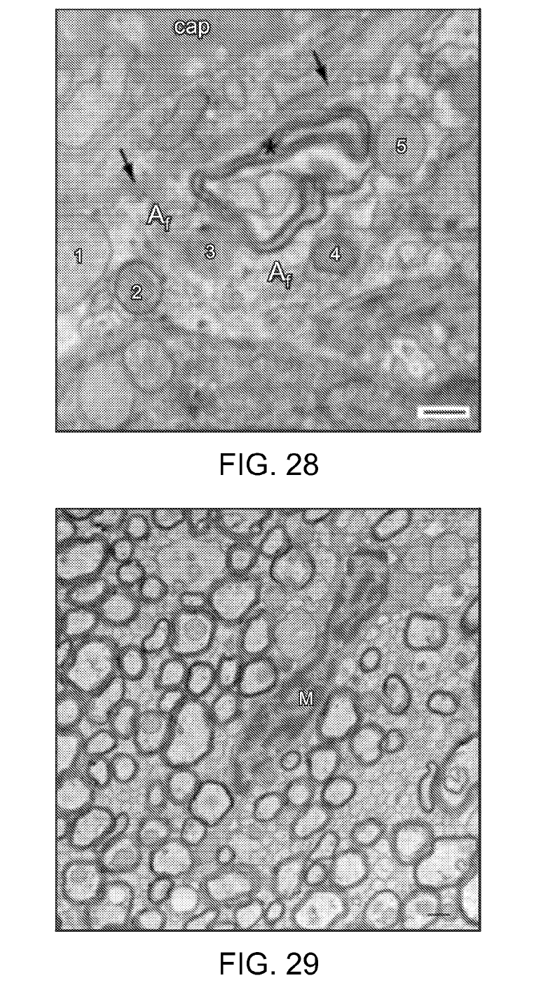

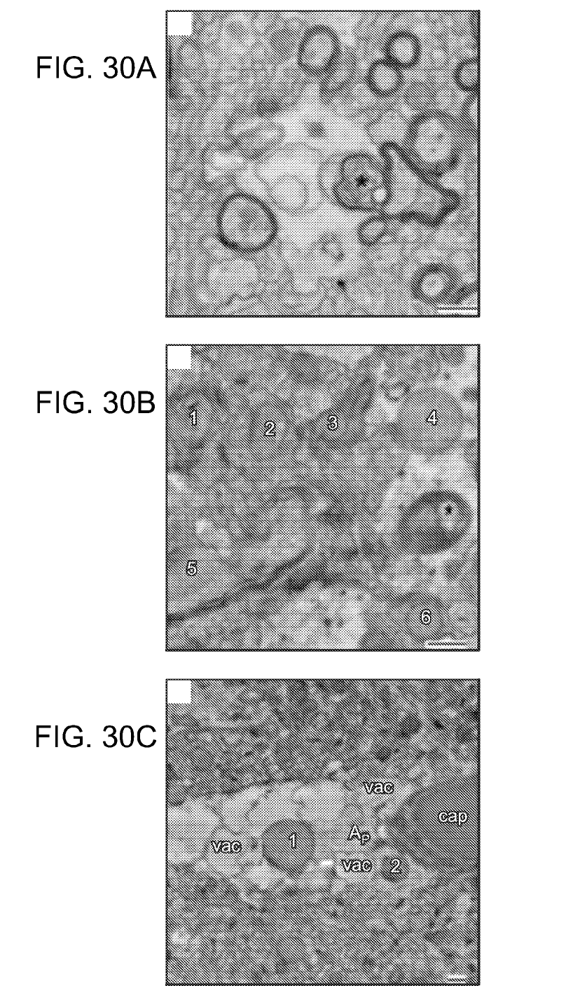

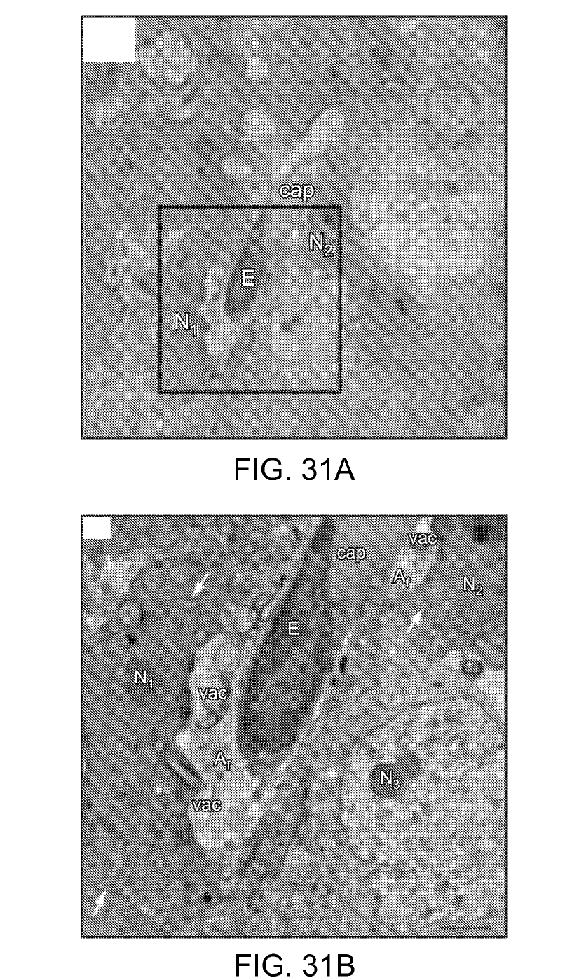

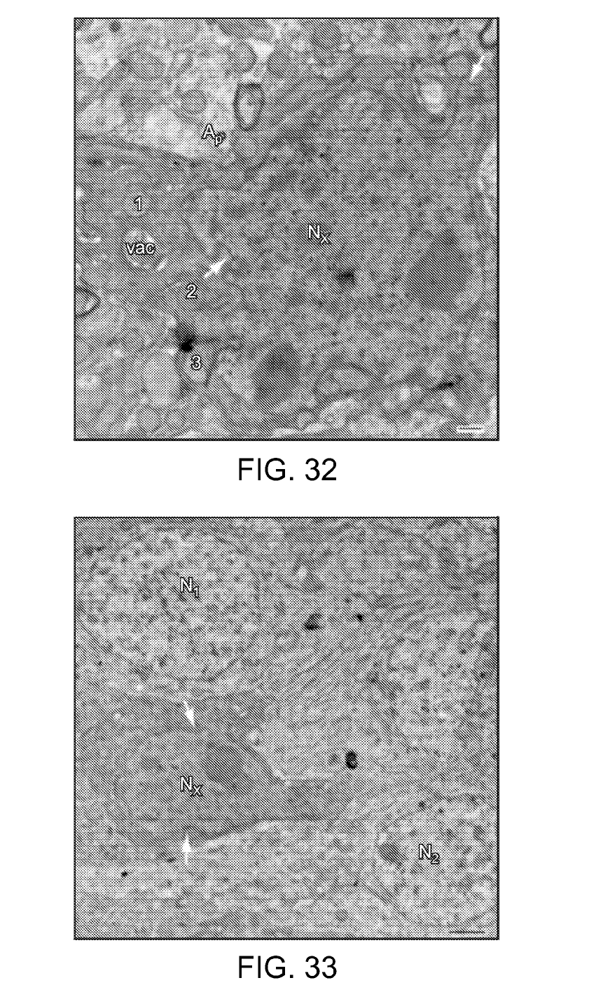

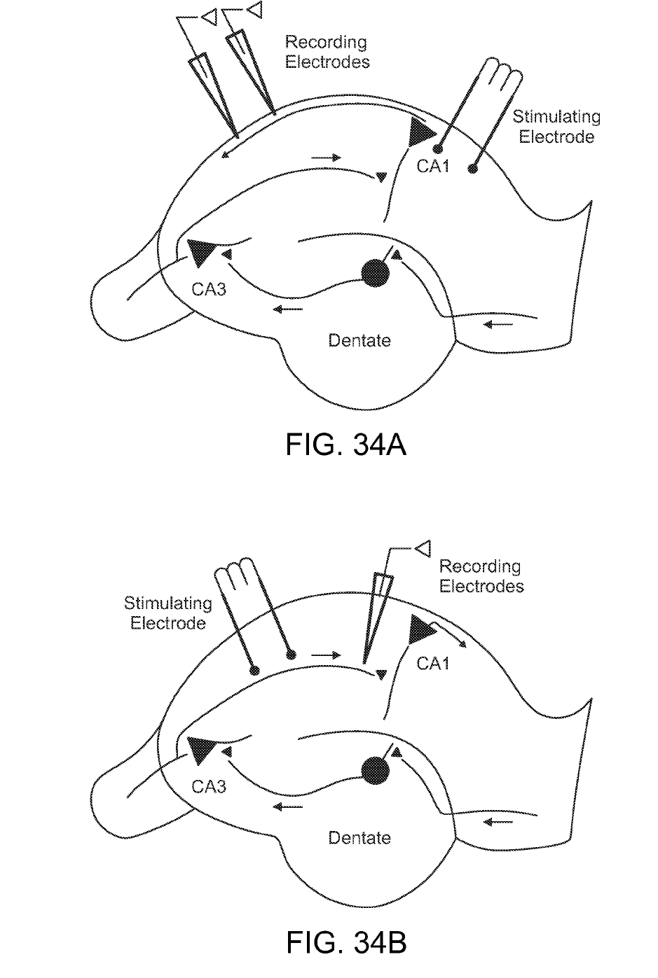

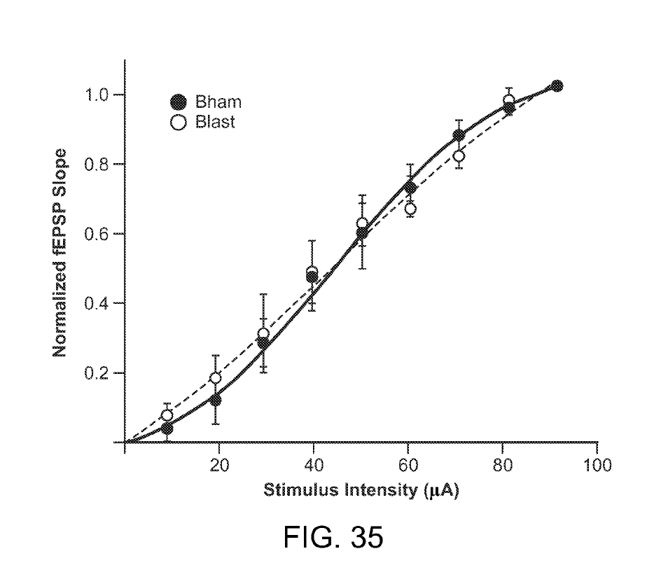

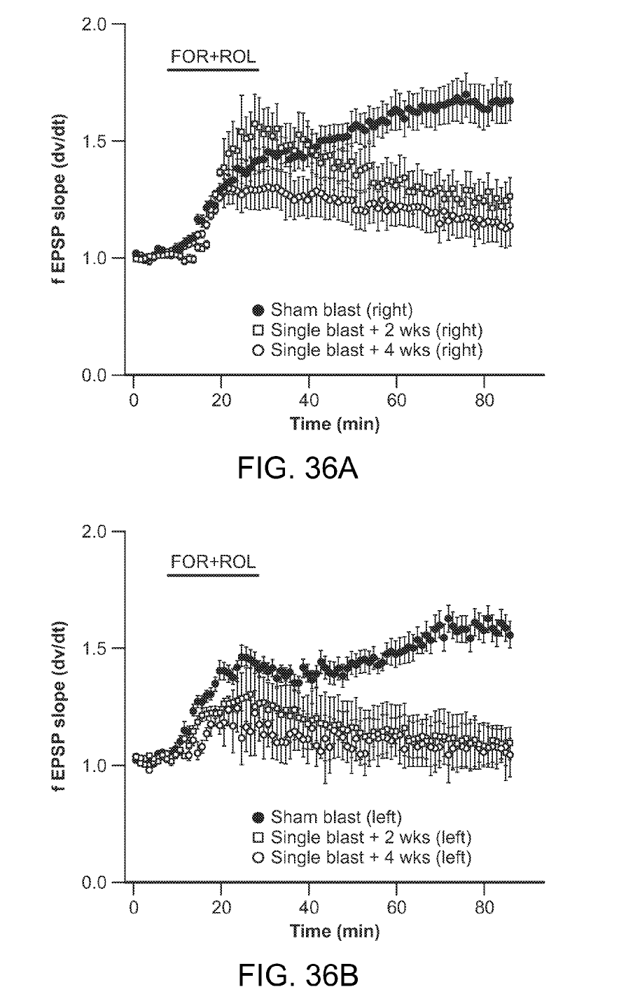

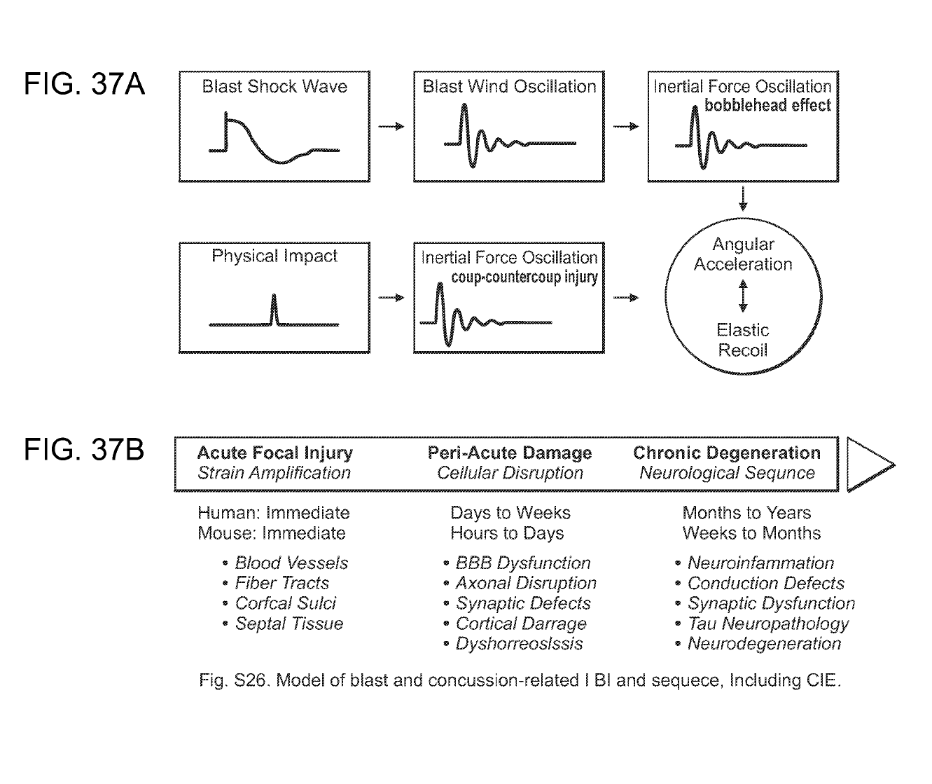

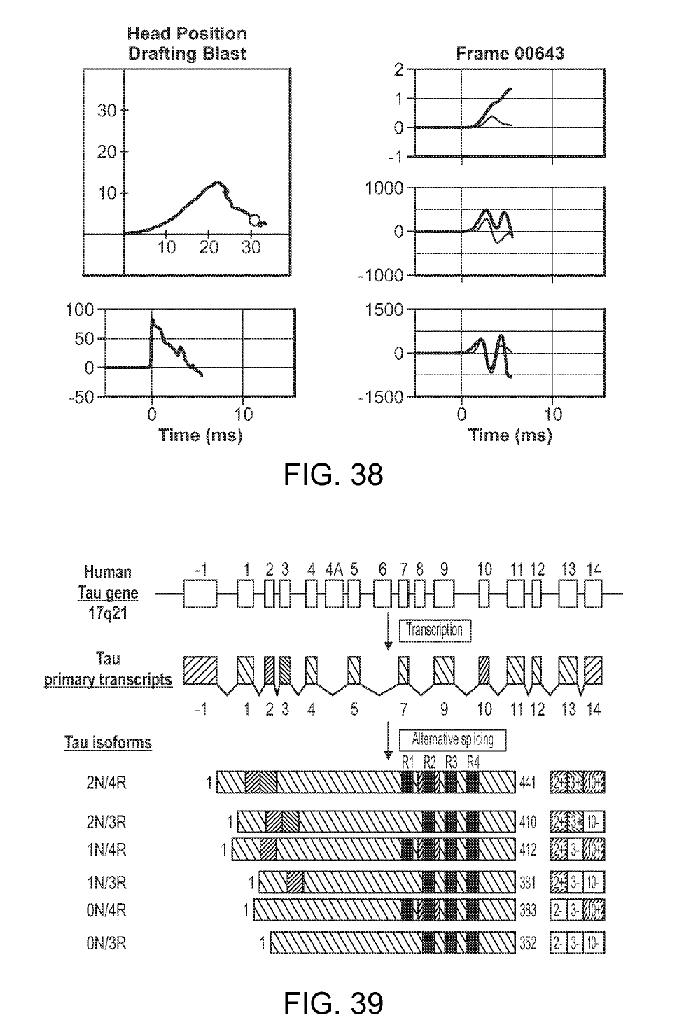

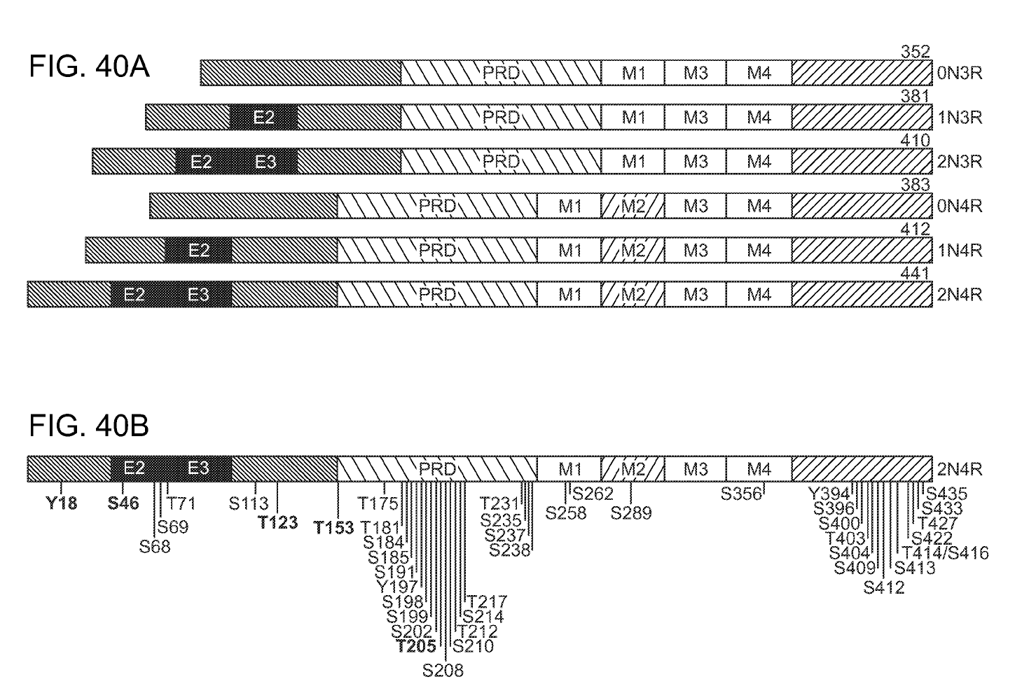

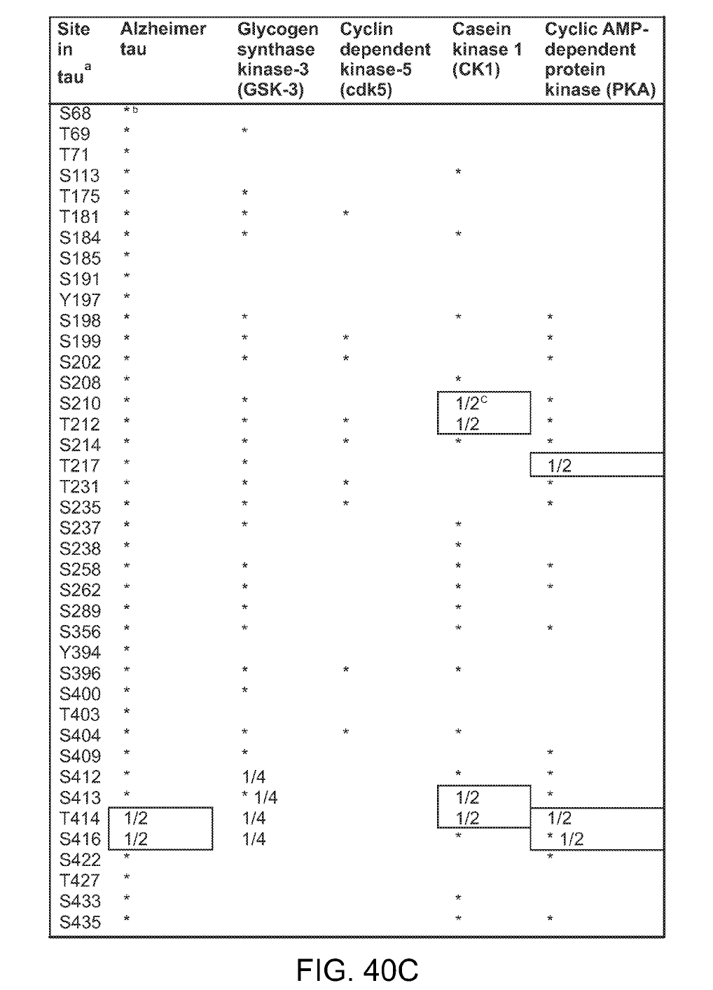

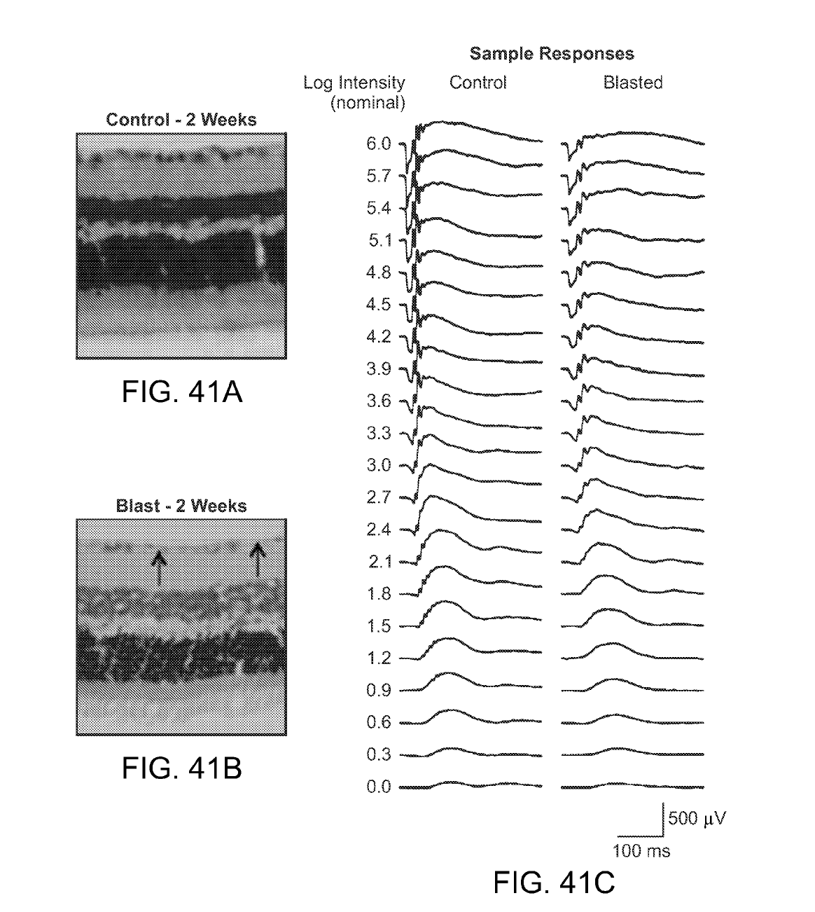

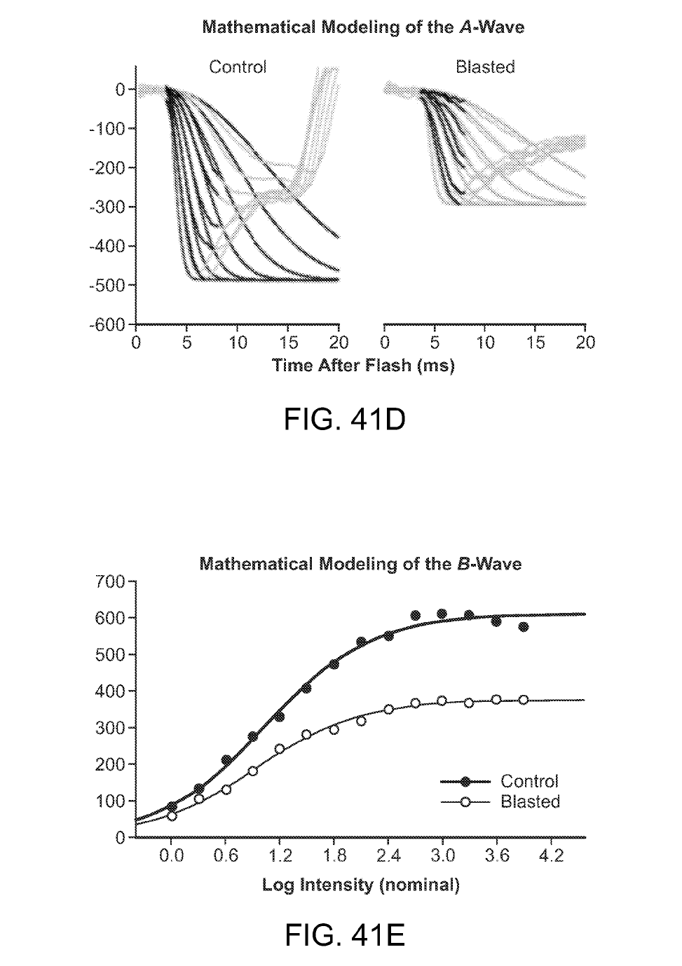

Head Fixation. Head fixation was accomplished using two miniature nylon cable ties with minimal face-on cross-sectional area. Prior to immobilization, the head was securely positioned on a rigid bite bar fixed to the in-tube restraint. The head was immobilized by positioning one band across the rostral aspect of the skull proximal to the incisor. The second band was placed immediately posterior to the caudalmost aspect of the skull. Neither band obstructed the oncoming blast shock wave (FIG. 8). Care was taken to avoid airway compromise. Thoracic protection was provided as described above. This immobilization procedure prohibited head displacement in all three Cartesian planes of motion during experimental blast.

Mouse Hippocampus Slice Electrophysiology. Mice were decapitated under deep isoflurane anesthesia and the brains quickly removed, hemisected, and blocked with a vibratome (DTK1000, Ted Pella, Co., Redding, Calif.) at a thickness of 350 μm. The tissue block was glued with cyanoacrylate adhesive to a stage immersed in ice-cold, oxygenated artificial cerebrospinal fluid (aCSF; NaCl, 126 mM; KCl, 3 mM; NaH₂PO₄, 1.25 mM; MgCl, 1.3 mM; CaCl², 2.5 mM; NaHCO³, 26 mM; glucose, 10 mM; saturated with 95% 02 and 5% CO²) maintained at 2-4° C., then placed in a conditioning chamber containing aCSF at room temperature for at least 1 hr before transfer to an interface chamber maintained at 32° C. for recording. Slices were perfused with aCSF during experiments. Experimental drugs were bath applied in the perfusate. For studies of Schaffer Collateral-CA1 synaptic transmission and plasticity, low resistance recording electrodes were pulled with a Flaming/Brown Micropipette puller (Model P-97, Sutter Instrument, Novato, Calif., USA) using thin-walled borosilicate glass (1-2 MΩ with aCSF; A-M Systems, Sequim, Wash.), and inserted into the stratum radiatum of the hippocampus CA1 field to record field excitatory post-synaptic potentials (fEPSPs). A bipolar stainless steel stimulating electrode was placed in Schaffer collateral-commissural fibers the stratum radiatum, and current pulses were applied with stimulus intensity adjusted to evoke approximately 50% of maximal fEPSPs (50 pA to 100 pA; 100 μs duration) at 30 s intervals. Electrical stimulation was delivered by an ISO-Flex isolator controlled by a Master eight-pulse generator (AMPI, Jerusalem, Israel) triggered by a Multiclamp 700B amplifier (Molecular Devices, Sunnyvale, Calif.), and signals were digitized and recorded using the Multiclamp 700B. fEPSP slope was measured by linear interpolation from 20-80% of maximum negative deflection, and slopes confirmed to be stable+10% for at least 15 min. Data were analyzed using Clampfit (Version 9, Molecular Devices, Sunnyvale, Calif.) on an IBM-compatible personal computer. Evoked fEPSPs (50% of maximum amplitude, 2-4 mV) were recorded in the apical dendritic field in stratum radiatum for a stable baseline period of at least 30 min and evoked by single square pulses (10-100 μA, 150 μs) applied at 30 s intervals from a bipolar stainless-steel stimulating electrode (FHC, Bowdoin, Me.). The high-frequency stimulus (HFS) paradigm for induction of homosynaptic LTP consisted of three theta burst trains, each train consisting of 10 bursts of 5 pulses each with a burst frequency of 100 Hz with interburst interval of 200 ms applied at 120 s intervals. For measurement of axonal conduction velocity, two extracellular recording electrodes were placed in CA1 stratum alveus approximately 200 μm apart, and a bipolar stimulating electrode placed 100 μm away from the nearest of the two recording electrodes to antidromically activate CA1 pyramidal neuron axons coursing through the stratum alveus. The latency differences of the peak negativity between the two recording electrodes and the spatial distance were used to calculate axonal conduction velocity for each slice.

Assessment of Hippocampal-Dependent Learning and Memory. Neurobehavioral assessment was performed using an open-field test and Barnes maze (Med-Associates, Inc., St. Albans, Vt., USA). Open-field testing to assess baseline locomotor functioning (average velocity), exploratory activity (total distance), and thigmotaxis (number of central zone entries) was performed by placing each animal subject in the middle of a 42.5 cm×42.5 cm open arena and monitoring movement for 10 min using a 3D infrared diode motion detector system (Any-Maze, Stoelting Co., Inc., Wood Dale, Ill.). Barnes maze evaluation was conducted using a 20-box apparatus with 900 lux surface light intensity. Animal subjects were familiarized with the test apparatus by placement on the platform and gentle guidance to the escape box. Training sessions were conducted across four training trials per day for four days. The order of testing of individual subjects was the same throughout daily sessions, but randomized across the four test days for a total of 16 trials. To initiate testing, a single mouse was placed in the start box in the middle of the maze and released. Test subjects were evaluated while locating a single escape box placed at a constant position. Spatial learning was assisted by distant visual cues that remained constant during across test sessions. Movement was tracked and recorded electronically. Latency to find the escape box, trajectory velocity to the escape box, and total trajectory distance was assessed and recorded daily. Memory retrieval was evaluated by replacing the escape box with a blank box 24 hours after the last training session. Memory retrieval was assessed by electronically recording the number of nose pokes into the blank box as a percentage of total nose pokes.

Quantitative Assessment of Phosphorylated and Total Tau Protein. For immunoblot analysis, left and right hemisected brain samples were obtained from PBS-perfused mice 2 weeks after exposure to a single shock tube blast (n=6 mice) or sham blast (n=6 mice). Snap frozen hemisected brain specimens were thawed and resuspended in 0.7 ml protease-phosphatase inhibitor buffer. Equal volumes of homogenized samples were subjected to standard polyacrylamide gel electrophoresis in duplicate and immunoblotted with monoclonal antibody AT270 (Innogenetics Inc., Alpharetta, Ga., USA) directed against tau protein phosphorylated at threonine-181 (pT181), monoclonal antibody CP-13 directed against tau protein phosphorylated at serine-202 (pS202), or monoclonal antibody Tau 5 directed at phosphorylation-independent tau protein (total tau). In order to compare the Tau 5 immunolabeling patterns between the experimental and control samples, triplicate densitometry measurements were conducted on each of the 3 tau isoform bands (maximum for each band) and summed. We used a commercial enzyme-linked immunosorbent assay (ELISA) kit to quantitate murine-specific tau protein phosphorylated at serine 199 (Invitrogen, Carlsbad, Calif., USA). Frozen brain samples were homogenized in eight volumes of 5 M guanidine-HCl 50 mM Tris (pH 8) followed by five passes in a glass teflon homogenizer. Homogenates were mixed for 3 hrs, diluted into PBS containing protease inhibitors, and centrifuged for 20 min at 16,000 g. Supernatants were diluted and assayed in quadruplicate for phosphorylated tau according to the manufacturer's instructions.

Statistical Analyses. Comparisons of axonal conduction velocity and LTP magnitude between sham-blast control mice and blast-exposed mice 14 and 28 days post-exposure were made using repeated-measures multi-factorial ANOVA with Bonferroni-Dunn post-hoc correction. Neurobehavioral assessment was conducted using an open-field test and Barnes maze (Med-Associates, St. Albans, Vt.). Longitudinal data were compared between blast-exposed mice and sham-blast controls using repeated measures ANOVA. Memory retrieval was evaluated by Student's t-test for two-tailed data. Immunoblot densitometry and biochemical data were evaluated by two-tailed Student's t-test. Levels of significance are indicated as follows: *, P<0.05; **, P<0.01; ***, P<0.001. Statistical significance was preset at P<0.05.

CTE Neuropathology in Blast-Exposed Military Veterans and Athletes with Repetitive Concussive Injury

We performed comprehensive neuropathological analyses (table S1) of postmortem brains obtained from a case series of military veterans with known blast exposure and/or concussive injury (n=4 males; ages 22 to 45 years; mean, 32.3 years). We compared these neuropathological analyses to those of brains from young amateur American football players and a professional wrestler with histories of repetitive concussive injury (n=4 males; ages 17 to 27 years; mean, 20.8 years) and brains from normal controls of comparable ages without a history of blast exposure, concussive injury, or neurological disease (n=4 males; ages 18 to 24 years; mean, 20.5 years). Case 1, a 45-year-old male U.S. military veteran with a single close-range IED blast exposure, experienced a state of disorientation without loss of consciousness that persisted for ˜30 min after blast exposure. He subsequently developed headaches, irritability, difficulty sleeping and concentrating, and depression that continued until his death 2 years later from a ruptured basilar aneurysm. His medical history is notable for a remote history of concussion associated with a motor vehicle accident at age 8 years. Case 2, a 34-year-old male U.S. military veteran without a history of previous concussive injury, sustained two separate IED blast exposures 1 and 6 years before death. Both episodes resulted in loss of consciousness of indeterminate duration. He subsequently developed depression, short-term memory loss, word-finding difficulties, decreased concentration and attention, sleep disturbances, and executive function impairments. His neuropsychiatric symptoms persisted until death from aspiration pneumonia after ingestion of prescription analgesics. Case 3, a 22-year-old male U.S. military veteran with a single close-range IED blast exposure 2 years before death. He did not lose consciousness, but reported headache, dizziness, and fatigue that persisted for 24 hours after the blast. He subsequently developed daily headaches, memory loss, depression, and decreased attention and concentration. In the year before his death, he became increasingly violent and verbally abusive with frequent outbursts of anger and aggression. He was diagnosed with posttraumatic stress disorder (PTSD) 3 months before death from an intracerebral hemorrhage. His past history included 2 years of high school football and multiple concussions from first fights. Case 4, a 28-year-old male U.S. military veteran with two combat deployments, was diagnosed with PTSD after his first deployment 3 years before death. His history was notable for multiple concussions as a civilian and in combat, but he was never exposed to blast. His first concussion occurred at age 12 after a bicycle accident with temporary loss of consciousness and pre/posttraumatic amnesia. At age 17, he experienced a concussion without loss of consciousness from helmet-to-helmet impact injury during football practice. At age 25, he sustained a third concussion during military deployment with temporary alteration in mental status without loss of consciousness. Four months later at age 26, he sustained a fourth concussion with temporary loss of consciousness and posttraumatic amnesia resulting from a motor vehicle-bicycle collision. Afterward, he experienced persistent anxiety, difficulty concentrating, word-finding difficulties, learning and memory impairment, reduced psychomotor speed, and exacerbation of PTSD symptoms. He died from a self-inflicted gunshot wound 2 years after his last concussion. The athlete group included Case 5, a 17-year-old male high school American football player who died from second impact syndrome 2 weeks after sustaining a concussion; Case 6, an 18-year-old high school American football and rugby player with a history of three to four previous concussions, one requiring hospitalization, who died 10 days after his last concussion; Case 7, a 21-year-old male college American football player, who played as a lineman and linebacker but had never been diagnosed with a concussion during his 13 seasons of play beginning at age 9, and who died from suicide; and Case 8, a 27-year-old male professional wrestler who experienced more than 9 concussions during his 10-year professional wrestling career who died from an overdose of OxyContin. The normal control group included Case 9, an 18-year-old male who died suddenly from a ruptured basilar aneurysm; Case 10, a 19-year-old male who died from a cardiac arrhythmia; Case 11, a 21-year-old male who died from suicide; and Case 12, a 24-year-old male who died from suicide.

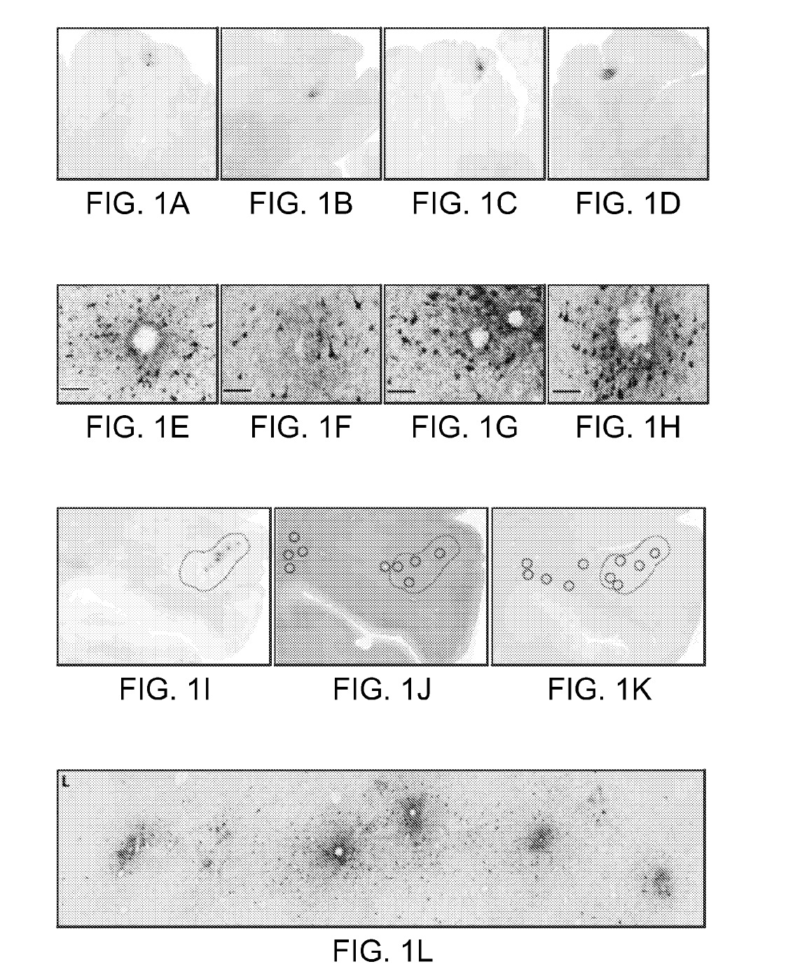

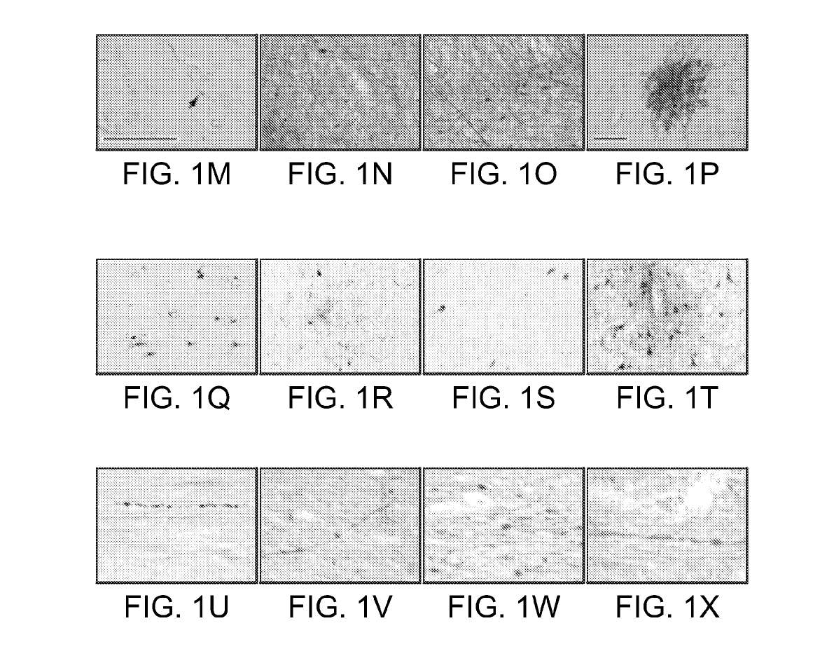

Neuropathological analysis of postmortem brains from military veterans with blast exposure and/or concussive injury revealed CTE-linked neuropathology characterized by perivascular foci of tau-immunoreactive neurofibrillary tangles (NFTs) and glial tangles in the inferior frontal, dorsolateral frontal, parietal, and temporal cortices with predilection for sulcal depths (FIGS. 1, A, B, E, F, and I to X). NFTs and dystrophic axons immunoreactive for monoclonal antibody CP-13 (FIGS. 1, A to I, L, Q, R, and U, and FIG. 12) directed against phosphorylated tau protein at Ser²⁰² (pS²⁰²) and Thr²⁰⁵ (pT²⁰⁵), monoclonal antibody AT8 (FIG. 1S) directed against phosphorylated tau protein at Ser²⁰² (pS²⁰²) and Thr²⁰⁵ (pT²⁰⁵), and monoclonal antibody Tau-46 (FIG. 1T) directed against phosphorylation-independent tau protein were detected in superficial layers of frontal and parietal cortex and anterior hippocampus. Evidence of axon degeneration, axon retraction bulbs, and axonal dystrophy were observed in the subcortical white matter subjacent to cortical tau pathology (FIGS. 1, M and U to X). Distorted axons and axon retraction bulbs were prominent in perivascular areas. Large clusters of LN3-immunoreactive activated microglia clusters (FIGS. 1, K and P) were observed in subcortical white matter underlying focal tau pathology, but not in unaffected brain regions distant from tau lesions. Neuropathological comparison to brains from young-adult amateur American football players (FIGS. 1, C, D, G, and H) with histories of repetitive concussive and subconcussive injury exhibited similar CTE neuropathology marked by perivascular NFTs and glial tangles with sulcal depth prominence in the dorsolateral and inferior frontal cortices. The young-adult athlete brains also revealed evidence of robust astrocytosis and multifocal axonopathy in subcortical white matter. Clusters of activated perivascular microglia were noted in the sub-cortical U-fibers. Neuropathological findings in the military veterans with blast exposure and/or concussive injury and young-adult athletes with repetitive concussive injury were consistent with our previous CTE case studies and could be readily differentiated from neuropathology associated with Alzheimer's disease, frontotemporal dementia, and other age-related neurodegenerative disorders. Control sections omitting primary antibody demonstrated no immunoreactivity. By contrast, none of the brains from the four young-adult normal control subjects demonstrated phosphorylated tau pathology, axonal injury, sub-cortical astrocytosis, or microglial nodules indicative of CTE or other neurodegenerative disease (FIG. 13).

### Blast Exposure Induces Traumatic Head Acceleration in a Blast Neurotrauma Mouse Model

We developed a murine blast neurotrauma model to investigate mechanistic linkage between blast exposure, CTE neuropathology, and neurobehavioral sequelae. Our compressed gas blast tube was designed to accommodate mice and allowed free movement of the head and cervical spine to model typical conditions associated with military blast exposure. Wild-type C57BL/6 male mice (2.5 months) were anesthetized and exposed to a single blast with a static (incident) pressure profile comparable in amplitude, waveform shape, and impulse to detonation of 5.8 kg of trinitrotoluene (TNT) at a standoff distance of 5.5 m and in close agreement with ConWep (Conventional Weapons Effects Program) (FIG. 2A). The model blast is comparable to a common IED fabricated from a 120-mm artillery round and is within the reported range of typical explosives, blast conditions, and standoff distances associated with military blast injury).

To investigate intracranial pressure (ICP) dynamics during blast exposure, we inserted a needle hydrophone into the hippocampus of living mice and monitored pressure dynamics during blast exposure. We detected blast wavefront arrival times in the brain that were indistinguishable from corresponding free-field pressure (FFP) measurements in air (FIG. 2B) and in close agreement with ConWep analysis of an equivalent TNT blast (FIG. 2A). To investigate possible thoracic contributions to blast-induced ICP transients, we evaluated pressure tracings in the hippocampus of intact living mice (FIG. 2B) and compared results to the same measurements obtained in isolated mouse heads severed at the cervical spine (FIG. 2C). Blast-induced pressure amplitudes in the two experimental preparations were comparable to each other and to the corresponding FFP measurements in air, after accounting for the addition of the dynamic pressure on the head. Small differences in the pressure waveforms were within the expected range given frequency-dependent response characteristics of the transducers and differences in the two experimental preparations. We did not detect delayed blast-induced ICP transients in either preparation over recording times up to 100 ms. These observations indicate that blast wavefront transmission in the mouse brain is mediated without significant contributions from thoracovascular or hydrodynamic mechanisms.

In our system, the blast shock wave traveling at ˜450 m/s encountered the left lateral surface of the mouse head first, then traversed the ˜11-mm skull width in ˜24 μs. The pressure differential associated with this traversal has an insignificant effect on skull displacement due to the short time interval. For the remainder of the waveform duration, the static pressures at the lateral surfaces of the skull are virtually identical and the corresponding transient effects are negligible. The air-skull impedance mismatch creates a back-reflected air shock as well as a rapidly moving (≧1500 m/s) transmitted shock wave, the latter taking a maximum of ˜7 μs to traverse the cranium and cranial contents. Although the reflected and transmitted shock waves are large (˜2.5 times greater than the 77-kPa incident overpressure), the ˜7-μs traversal time of the skull-brain transmitted wave is short enough to allow rapid equilibration across the skull. Thus, the head acts acoustically as a “lumped element” (Blackstock et al., in Fundamentals of Physical Acoustics. (Wiley & Sons, New York, N.Y. 2000), pp. 146-150; Cloots et al., 2011 Biomech Model Mechanobiol 10: 413-422). The only significant pressure term remaining is the ˜19-kPa peak dynamic pressure generated by blast wind. We concluded that an ICP transducer in the brain parenchyma should measure pressure differentials that do not differ by more than 19 kPa from FFP values, at least beyond the initial 30 μs after blast arrival. This analysis was confirmed by experimental measurements (FIG. 2B). Only the initial rise of the blast wave has a short enough time scale to be affected by propagation effects in the head, a prediction confirmed by the longer rise time of the ICP compared to the static FFP waveforms (FIGS. 2, B and C). The remaining waveform components evenly distribute through the brain with amplitude and shape that approximate the static FFP (FIG. 2A).

The blast wave had a measured Mach number of 1.26±0.04, from which the calculated blast wind velocity was 150 m/s (336 miles/hour). Kinematic analysis of high-speed videographic records of head movement during blast exposure confirmed rapid oscillating acceleration-deceleration of the head in the horizontal and sagittal planes of motion (FIG. 2, D to G). We calculated peak average radial head acceleration of 954±215 krad/s² (FIG. 2G), corresponding to 100.2 N exerted on the head during blast exposure. Peak angular and centripetal acceleration were most significant during the positive phase of the blast shock wave. No appreciable head acceleration was detected after ˜8 ms.

### Single-Blast Exposure Induces CTE-Linked Neuropathology, Ultrastructural Pathology, and Phosphorylated Tau Proteinopathy in a Blast Neurotrauma Mouse Model

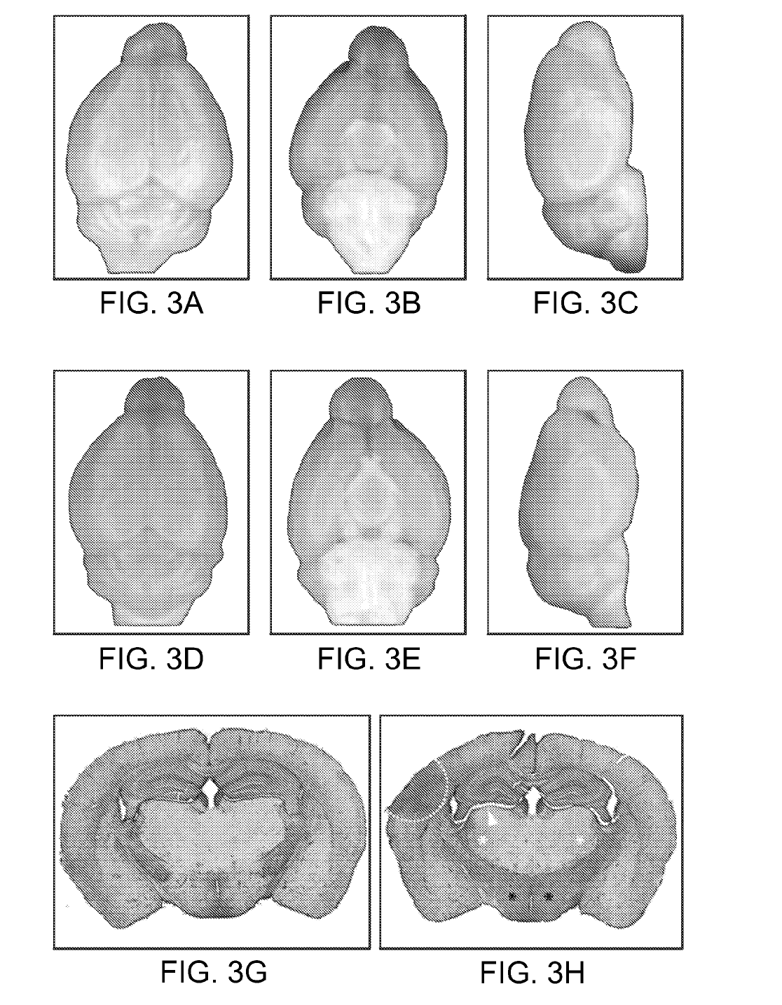

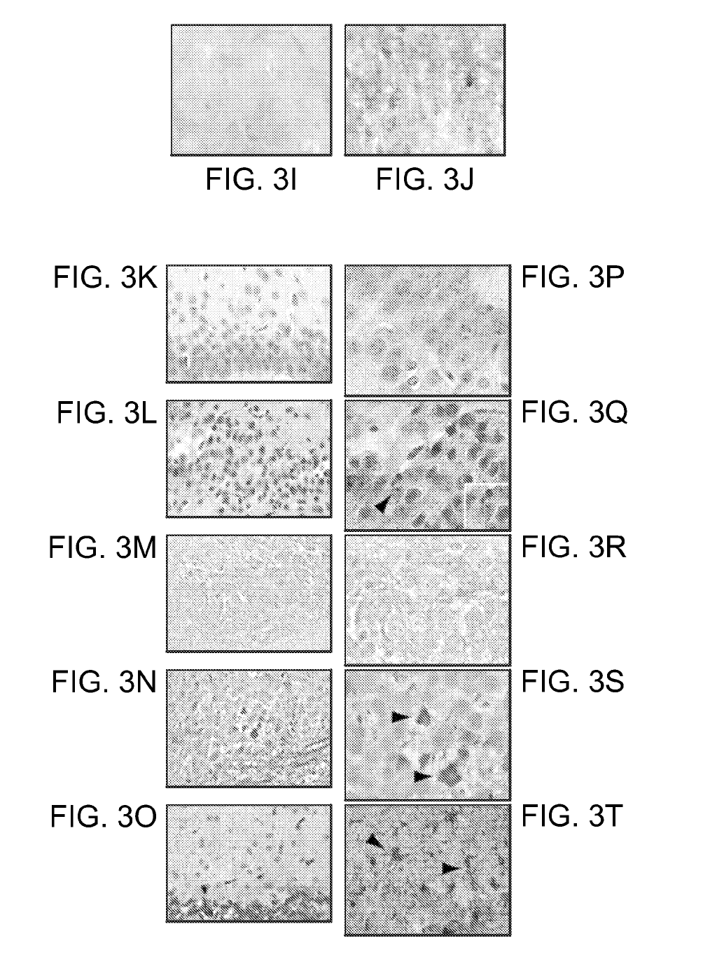

We hypothesized that blast forces exerted on the skull would result in head acceleration-deceleration oscillation of sufficient intensity to induce persistent brain injury (“bobblehead effect”). To evaluate this hypothesis, we studied brains from mice euthanized 2 weeks after exposure to a single blast or sham blast. Gross examination of postmortem brains from both groups of mice was unremarkable and did not reveal macroscopic evidence of contusion, necrosis, hematoma, hemorrhage, or focal tissue damage (FIG. 3, A to F, and FIG. 19). In contrast, brains from blast-exposed mice showed marked neuropathology by immunohistological analysis (FIGS. 3, H, J, L, Q, N, S, and T). Blast-exposed brains exhibited robust reactive astrocytosis throughout the cerebral cortex, hippocampus, brainstem, internal capsule, cerebellum, and corticospinal tract (FIGS. 3, H and T) that was not observed in brains from sham-blast control mice (FIGS. 3, G and O). Brains from blast-exposed mice also exhibited enhanced somatodendritic phosphorylated tau CP-13 immunoreactivity in neurons in the superficial layers of the cerebral cortex (FIG. 3J) that was not observed in the brains of sham-blast control mice (FIG. 3I). The cerebral cortex and CA1 field of the hippocampus in the brains of blast-exposed mice were also notable for clusters of chromatolytic and pyknotic neurons with nuclear and cytoplasmic smudging and beaded, irregularly swollen dystrophic axons (FIGS. 3, L and Q) that were not observed in the brains of sham-blast control mice (FIGS. 3, K and P). Hippocampal CA1 neurons in blast-exposed mice were intensely Tau-46-immunoreactive (FIGS. 3, N and S) compared to sham-blast controls (FIGS. 3, M and R) and additionally showed evidence of frank neurodegeneration in the hippocampal CA1 and CA3 subfields and dentate gyms (FIG. 4 and FIG. 20). Activated perivascular microglia were observed throughout the brain in blast-exposed mice and were especially notable in the cerebellum (FIG. 3T; compared to control, FIG. 3O). Patchy loss of cerebellar Purkinje cells with empty baskets was also noted in blast-exposed mice but not in sham-blast control mice. Examination of the cervical spinal cords of blast-exposed mice did not reveal evidence of motor neuron dropout or degeneration (FIGS. 21, A and B). However, blast-exposed mice did show decreased choline acetyltransferase immunoreactivity in motor neurons in the cervical cord (FIG. 21D) and cranial nerve XII (FIG. 21F) when compared to sham-blast controls (FIGS. 21, C and E), suggesting loss of central cholinergic inputs.

Ultrastructural pathology was observed in electron micrographs of neurons, axons, and capillaries in the hippocampi of blast-exposed mice but not in sham-blast control mice (FIG. 4 and FIGS. 22-33). Examination of semithick sections of hippocampus CA1 and CA3 regions and dentate gyms in brains from blast-exposed mice revealed clusters of chromatolytic and pyknotic neurons throughout the stratum pyramidale and a marked paucity of dendritic profiles in the stratum radiatum (FIG. 4H and FIGS. 20 B and C) that was not evident in the brains of sham-blast control mice (FIG. 4A and FIG. 20A). Blast-related ultrastructural micro-vascular pathology was notable for the presence of hydropic perivascular astrocytic end-feet (FIGS. 4, I and J, and FIGS. 22, 24-27, 30C, and 31). Pathologically swollen, edematous, and often highly vacuolated astrocytic end-feet were observed in association with dysmorphic capillaries marked by pathologically thickened, tortuous basal lamina and abnormal endothelial cells with irregularly shaped nuclei (FIG. 4L and FIGS. 22-27). Perivascular processes in the hippocampi of blast-exposed mice often contained inclusion bodies, lipofuscin granules, myelin figures, and autophagicvacuoles (FIGS. 4, I, L, and N, and FIGS. 22, 23, 25, and 28-30). Pericytes (FIGS. 4, I and L, and FIGS. 22, 23, 25, 28), microglial cells (FIG. 29), dystrophic myelinated nerve fibers (FIG. 4K and FIGS. 26, 28, 30A), and “dark neurons” (FIG. 4M and FIGS. 31-33) with electron-dense cytoplasm and irregularly shaped nuclei were frequently observed in proximity to these abnormal capillaries in blast-exposed mice. By contrast, the brains of sham-blast control mice exhibited normal hippocampal cytoarchitecture without evidence of ultrastructural neuropathology (FIG. 4, A to G).

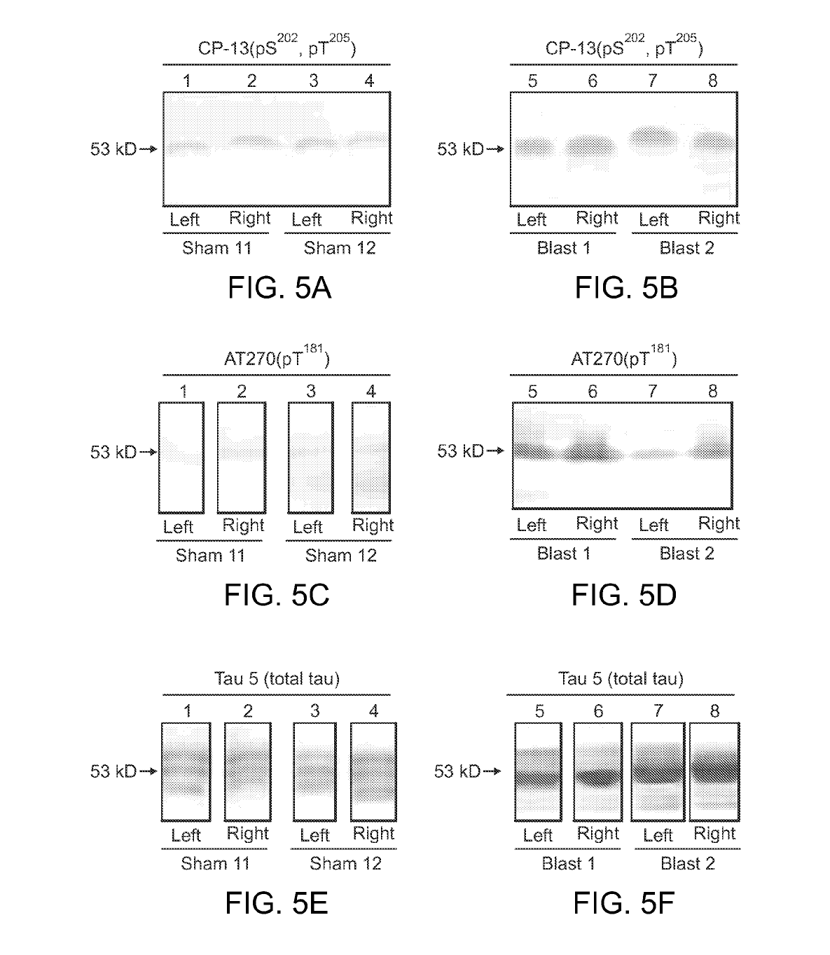

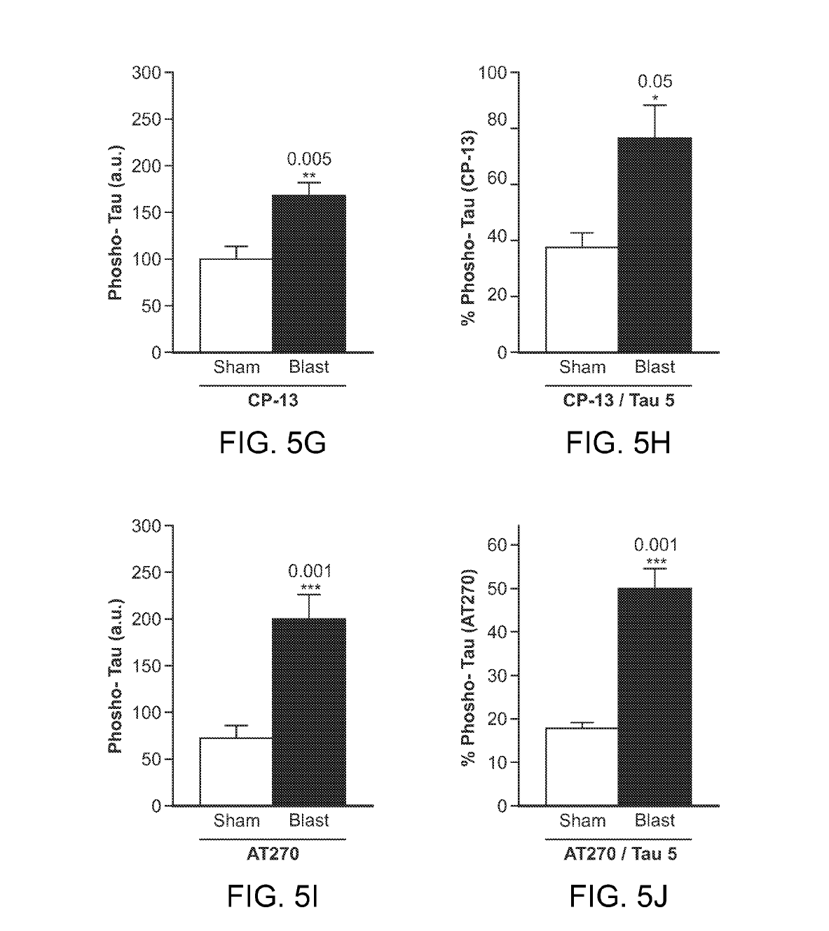

To confirm the presence of phosphorylated tau proteinopathy in the brains of blast-exposed mice, we performed immunoblot analysis of tissue homogenates prepared from brains harvested from mice 2 weeks after single-blast or sham-blast exposure (FIG. 5). Immunoblot analysis revealed a significant blast-related elevation of phosphorylated tau protein epitopes pT¹⁸¹ and pS²⁰² detected by monoclonal antibody CP-13 (FIGS. 5, A, B, and G) and pT²⁰⁵ detected by monoclonal antibody AT²⁷⁰ (FIGS. 5, C, D, and I) that are associated with early neurodegenerative tau misprocessing. Blast-related tau phosphorylation was also detected when quantitated as a ratio of phosphorylated tau protein to total tau protein (FIGS. 5, E, F, H, and J). In mice exposed to sham blast, all three of the major native murine tau isoforms (4R2N, 4R0N, and 4R1N) were evident (FIG. 5E). By contrast, immunoblots of brain homogenates prepared from mice exposed to a single blast revealed a tau protein isoform distribution pattern that was dominated by a single band corresponding to the intermediate-sized native tau isoform (4R1N; FIG. 5F). Phosphorylated tauopathy (FIGS. 5, B and D) and tau isoform distribution abnormalities (FIG. 5F) were detected bilaterally, a finding consistent with blast-related CTE neuropathology and electrophysiological deficits. Blast-induced brain tau proteinopathy was confirmed by enzyme-linked immunosorbent assay (ELISA) analysis of tau protein phosphorylated at pSer¹⁹⁹ (single blast, 40±2 ng/ml; sham blast, 31±2 ng/liter; P=0.027, two-tailed Student's t test).

### Single-Blast Exposure Persistently Impairs Axonal Conduction and Long-Term Potentiation of Activity-Dependent Synaptic Transmission in the Hippocampus

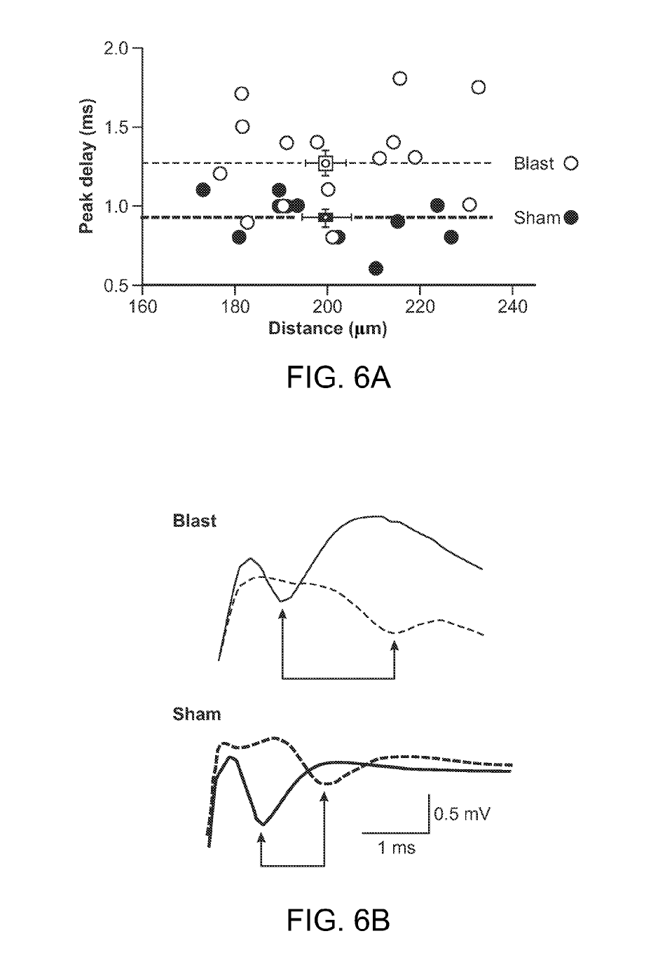

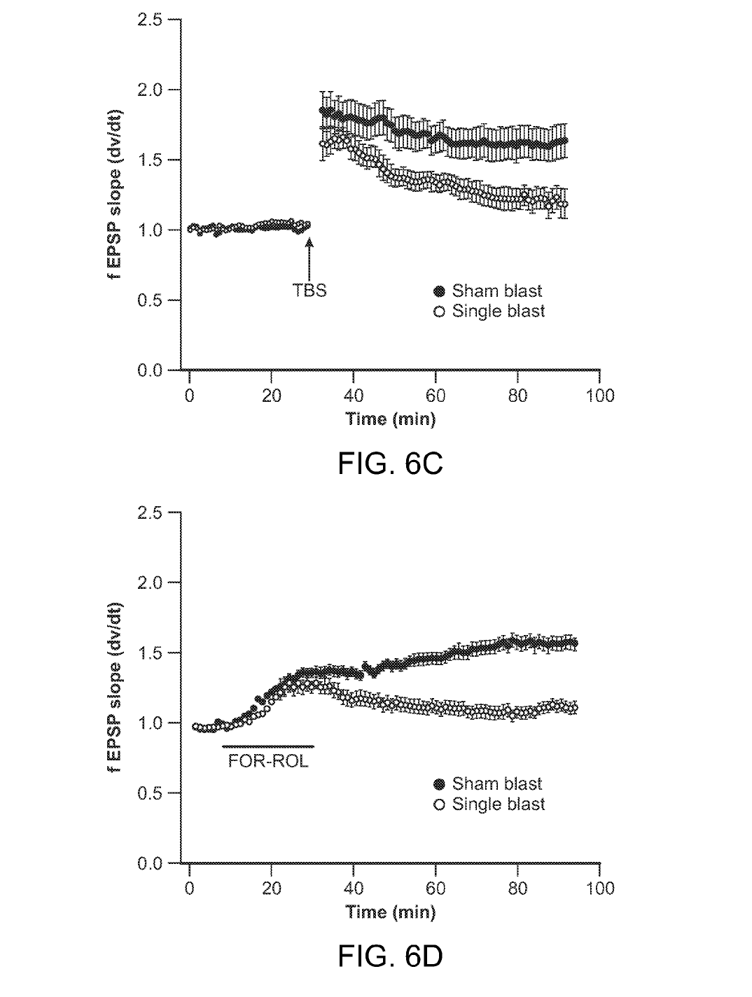

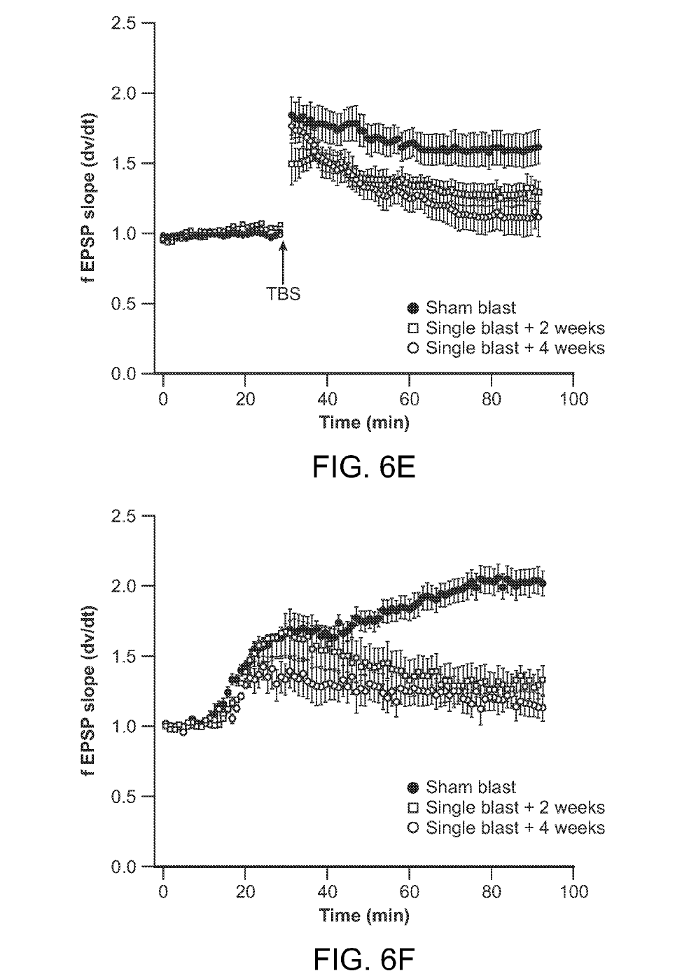

We investigated the possibility that blast-related histopathological and ultrastructural abnormalities would be reflected in equally persistent functional impairments in hippocampal neurophysiology. Analysis of Schaffer collateral-evoked synaptic field potential input-output relations (FIG. 34B) did not reveal an effect of blast exposure on baseline synaptic transmission at either 2 weeks or 1 month after blast exposure. However, axonal conduction velocity of CA1 pyramidal cell compound action potentials in the stratum alveus (FIG. 34A) was significantly slowed 2 weeks after blast exposure, an effect that persisted for at least 1 month [FIGS. 6, A and B; P<0.05, repeated-measures multifactorial analysis of variance (ANOVA)].

Next, we examined the effect of blast exposure on stimulus- and cyclic adenosine monophosphate (cAMP)-evoked long-term potentiation (LTP) of synaptic strength at Schaffer collateral-CA1 synapses (FIG. 34B), candidate mechanisms of memory storage. We found marked impairments of stimulus-evoked LTP in mouse slices prepared 2 weeks and 1 month after blast exposure (FIG. 6C; P<0.05, repeated-measures multifactorial ANOVA). When the 2-week and 1-month blast-exposed cohorts were examined independently, we found that the magnitude of posttetanic potentiation (PTP) immediately after application of theta-burst stimulation (TBS) was significantly less at the 2-week time point (FIG. 6E; P<0.05, repeated-measures multifactorial ANOVA). Although PTP recovered by 1 month after blast, the magnitude of LTP 1 hour after tetanus was significantly reduced at both postblast time points (FIG. 6E; P<0.05, repeated-measures multifactorial ANOVA). These results indicate that exposure to single blast impaired long-term activity-dependent synaptic plasticity for at least 1 month after blast exposure in our model. Next, we examined cAMP-dependent LTP of Schaffer collateral-CA1 field excitatory postsynaptic potentials (fEPSPs) induced by 15-min bath application of the adenylate cyclase activator forskolin (50 μM) plus the type II phosphodiesterase inhibitor rolipram (10 μM). In contrast to control slices, cAMP-LTP was profoundly attenuated 30 to 60 min after drug washout in hippocampal slices prepared from both left and right hemispheres of mice 2 weeks and 1 month after blast exposure (FIG. 6D and FIGS. 36 A and B; P<0.05, repeated-measures multifactorial ANOVA). As with stimulus-evoked LTP, cAMP-LTP was equally impaired at both 2 weeks and 1 month after blast exposure, demonstrating the long-term nature of blast effects on both activity-dependent and chemically evoked synaptic plasticity (FIG. 6F; P<0.05, repeated-measures multifactorial ANOVA).

Single-Blast Exposure Induces Long-Term Behavioral Deficits that are Prevented by Head Immobilization During Blast Exposure

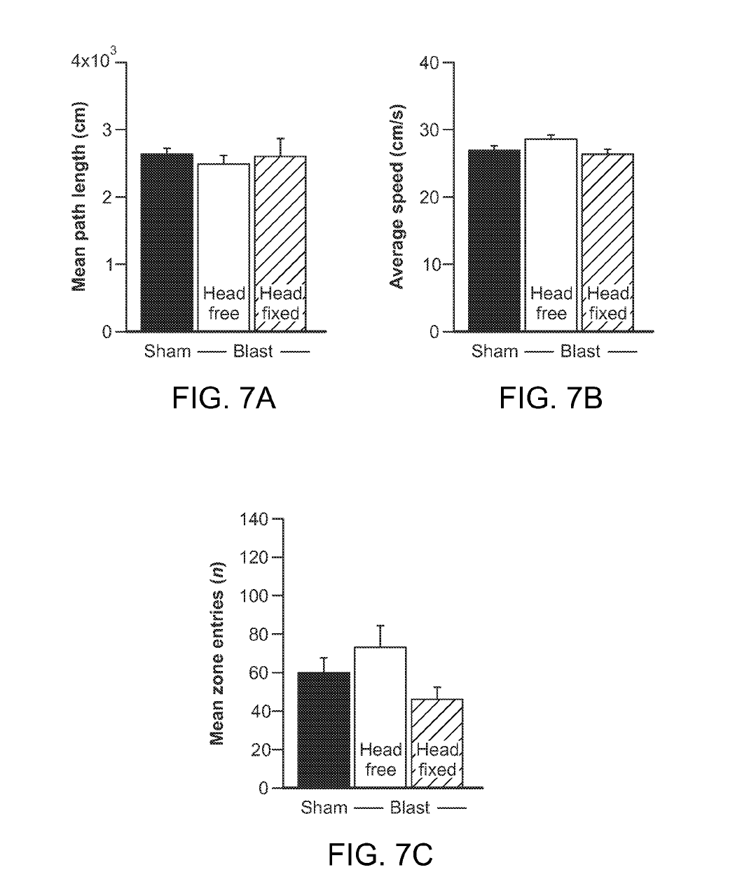

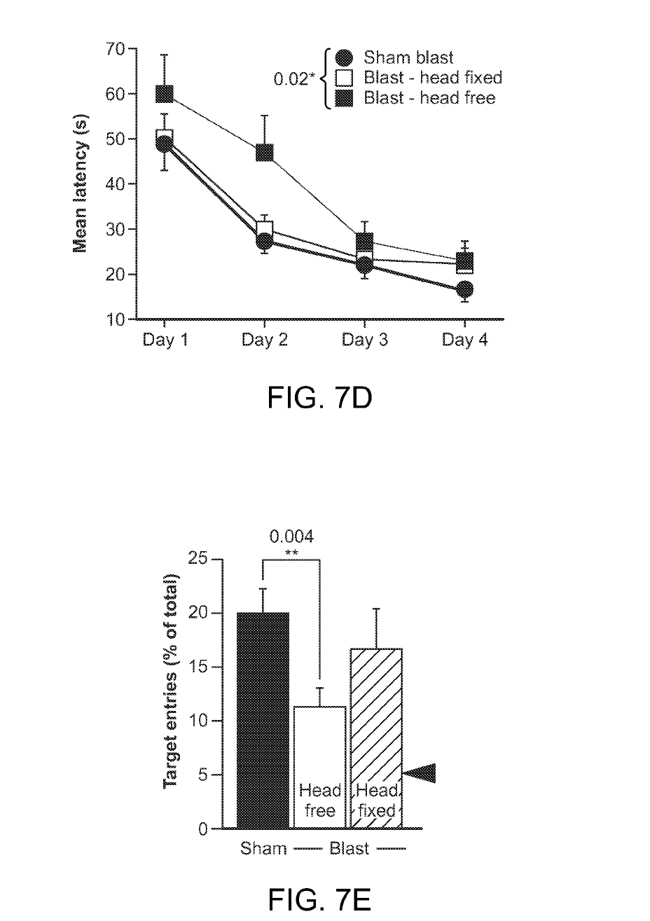

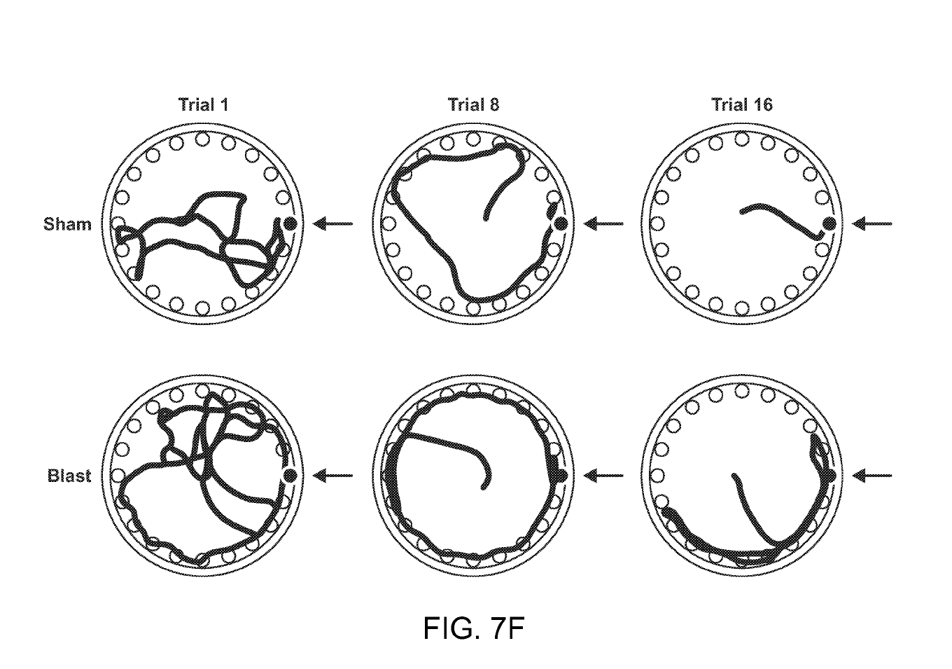

We did not detect significant differences between single-blast and sham-blast mice in total distance, mean velocity, or central zone entries in open-field behavior testing (FIG. 7, A to C), indicating that blast exposure did not impair gross neurological functioning with respect to locomotion, exploratory activity, and thigmotaxis (an indicator of murine anxiety assessed by movement close to the wall of the experimental apparatus). In contrast, when we tested acquisition and long-term retention of hippocampal-dependent spatial learning and memory in the Barnes maze (FIG. 7, D to F), we observed that blast-exposed mice exhibited significantly longer escape latencies (FIG. 7D; P<0.05, two-way ANOVA) and poorer memory retrieval 24 hours after the final training session (FIG. 7E; P<0.05, Student's t test) compared to sham-blast control mice. These findings are consistent with persistent blast-related hippocampal dysfunction.

The results of kinematic analysis (FIG. 2, D to G) suggested that blast-induced head acceleration was a likely pathogenic mechanism by which blast exposure leads to TBI and neurobehavioral sequelae. To test this hypothesis, we compared hippocampal-dependent learning acquisition and memory retention in mice with and without head immobilization during single-blast exposure and in sham-blast control mice. Head immobilization during blast exposure eliminated blast-related impairments in hippocampal-dependent learning acquisition (FIG. 7D; P>0.20, repeated-measures ANOVA with post hoc Scheffe test compared to sham-blast controls) and restored blast-related memory retention deficits to normal levels (FIG. 7E; P>0.20, one-way ANOVA with post hoc Scheffe test), supporting the conclusion that head acceleration is necessary for behavioral learning impairments.

### Blast Brain: An Invisible Injury Revealed

TBI is the “signature” injury of the conflicts in Afghanistan and Iraq and is associated with psychiatric symptoms and long-term cognitive disability. Recent estimates indicate that TBI may affect 20% of the 2.3 million U.S. servicemen and women deployed since 2001. CTE, a tau protein-linked neurodegenerative disorder reported in athletes with multiple concussions, shares clinical features with TBI in military personnel exposed to explosive blast. However prior to the invention, the connection between TBI and CTE has not been explored in depth. The studies described herein. investigate this connection in a case series of postmortem brains from U.S. military veterans with blast exposure and/or concussive injury. They report evidence for CTE neuropathology in the military veteran brains that is similar to that observed in the brains of young amateur American football players and a professional wrestler. The investigators developed a mouse model of blast neurotrauma that mimics typical blast conditions associated with military blast injury and discovered that blast-exposed mice also demonstrate CTE neuropathology, including tau protein hyperphosphorylation, myelinated axonopathy, microvascular damage, chronic neuroinflammation, and neurodegeneration. Surprisingly, blast-exposed mice developed CTE neuropathology within 2 weeks after exposure to a single blast. In addition, the neuropathology was accompanied by functional deficits, including slowed axonal conduction, reduced activity-dependent long-term synaptic plasticity, and impaired spatial learning and memory that persisted for 1 month after exposure to a single blast. The investigators then showed that blast winds with velocities of more than 330 miles/hour-greater than the most intense wind gust ever recorded on earth-induced oscillating head acceleration of sufficient intensity to injure the brain. The researchers then demonstrated that blast-induced learning and memory deficits in the mice were reduced by immobilizing the head during blast exposure. These findings provide a direct connection between blast TBI and CTE and indicate a primary role for blast wind-induced head acceleration in blast-related neurotrauma and its aftermath. This study also validates a blast neurotrauma mouse model that is useful for developing diagnostics, therapeutics, and rehabilitative strategies for treating blast-related TBI and CTE.

We analyzed a case series of postmortem human brains from U.S. military veterans with blast exposure and/or concussive injury and compared them to brains from young-adult athletes with histories of concussive injury and from normal controls of comparable ages without histories of blast exposure, concussive injury, or neurological disease. We uncovered evidence of CTE-linked tau neuropathology, including multifocal perivascular foci of neurofibrillary and glial tangles immunoreactive for phosphorylation-independent (Tau-46) and phosphorylation-dependent (CP-13) tau epitopes (McKee et al., 2010, J Neuropathol Exp Neurol 69: 918-929), in the brains of blast-exposed and/or concussive-injured veterans. This blast-associated CTE-linked tau neuropathology was indistinguishable from the tau neuropathology, neuroinflammation, and neurodegeneration observed in the brains of young-adult athletes with histories of repeat concussive injury. Examination of brains from wild-type C57BL/6 mice 2 weeks after exposure to a single controlled blast also revealed histopathological, ultrastructural, and biochemical evidence of CTE-linked neuropathology, including tau protein-linked immunoreactivity, persistent perivascular pathology, cortical and hippocampal neurodegeneration, myelinated axonopathy, chronic neuroinflammation with widespread astrocytosis and microgliosis, and phosphorylated tau proteinopathy. Overall, our findings of persistent CTE-linked neuropathology in the brains of military veterans with blast exposure and/or concussive injury and young athletes with repeat concussive injury suggest that TBI induced by different insults under different conditions can trigger common pathogenic mechanisms leading to similar neuropathology and sequelae. Notably, within this controlled case series, the effects of blast exposure, concussive injury, and mixed trauma (blast exposure and concussive injury) were indistinguishable.

Experimental results from our murine blast neurotrauma model provide evidence linking blast exposure with development of CTE-like tau neuropathology. Moreover, this blast-related neuropathology was associated with persistent neurophysiological and cognitive deficits that recapitulate clinical signs and symptoms reported in military veterans with blast-related TBI and concussive-injured athletes diagnosed with CTE. Exposure to a single blast in our mouse model was sufficient to induce early CTE-like neuropathology, slowed axonal conduction velocity, and defective stimulus- and cAMP-dependent LTP of synaptic transmission. These blast-related neurophysiological abnormalities were contemporaneous with somatodendritic alterations in hippocampal and cortical total tau and phosphorylated tau neuropathology and biochemistry, micro-vascular ultrastructural pathology, and impairment in hippocampal dependent learning acquisition and memory retention.

Although blast-exposed C57BL/6 mice recapitulated key features of human CTE neuropathology, including cellular accumulation of phosphorylated tau protein and pre-tangle tau protein neuropathology, it is notable that mature NFTs were not detected in the cortex or hippocampus of blast-exposed mice. This apparent discordance with human CTE neuropathology may be explained by the early time points chosen for evaluation in our mouse studies or, alternatively, as a forme fruste resulting from resistance of wild-type murine tau protein to form neurotoxic aggregates in vivo. However, our results demonstrate blast-related immunohistochemical and biochemical abnormalities in tau hyperphosphorylation at the 2-week time point after single-blast exposure. Studies of triple-transgenic mice expressing human tau protein and human amyloid-β peptide have shown that controlled cortical impact injury leads to rapid accumulation of hyperphosphorylated tau within 24 hours after experimental injury. These findings suggest that genotypic determinants may be critical factors that modulate temporal and phenotypic expression of TBI and late-emerging sequelae, including CTE.

ICP dynamics recorded during blast exposure revealed blast-induced pressure transients in the hippocampus that were coincident with and comparable in amplitude, waveform, and impulse to FFP measurements outside the cranium. This finding is consistent with the head acting as a lumped element for which the blast-induced external pressure differential equilibrates within ˜100 μs. Measured blast pressure amplitudes in the brain were on the order of 100 kPa (˜1 bar), a magnitude equivalent to water pressure at a depth of ˜10 m. Although it is possible that high-frequency components (>100 kHz) could lead to localized focusing due to reverberation and constructive interference, the pressure amplitudes we measured were far below tissue damage thresholds. Tissue damage associated with clinical ultrasound requires negative acoustic pressures in excess of 1 MPa that lead to excitation of cavitation bubbles. Thresholds for positive pressures are not well characterized but are likely to exceed 40 MPa because positive pressures commonly used in clinical shock wave lithotripsy are not associated with significant, if any, tissue damage. Thresholds for tissue damage from underwater sonar require ˜100 kPa and result from many cycles of bubble growth and collapse over tens of seconds of continuous wave excitation. Tissue damage in this setting is due to the negative pressure rather than exposure to a single compression pulse. These considerations indicate that direct tissue damage resulting from transmission of the blast shock wave through the brain is unlikely. Our results indicate that ICP transients closely approximate FFP measurements in air. Moreover, blast wavefront transmission was identical when measured in the brain of intact living mice or isolated mouse heads severed at the cervical spine, suggesting that neither thoracic-mediated mechanisms nor vascular hemodynamic effects contributed significantly to ICP transients during blast exposure. Together, our findings point to the substantial inertial forces and oscillating acceleration-deceleration cycles imposed on the head by blast wind (bobblehead effect) as the primary biomechanical mechanism by which blast exposure initiates acute closed-head brain injury and sequelae, including CTE (FIG. 37).

Here, we describe CTE-linked neuropathology in the brains of military veterans with blast exposure and/or concussive injury, young-adult athletes with repetitive concussive injury, and mice subjected to a single blast. These observations are consistent with a common injury mechanism involving oscillating head acceleration-deceleration cycles (bobblehead effect; FIG. 37) that lead to pathogenic shearing strain imposed on the cranial contents. Our observation that head immobilization during blast exposure prevented hippocampal-dependent learning and memory deficits in blast-exposed mice provides additional support for this injury mechanism and postulated relationship to persistent neurobehavioral sequelae. Recent studies have identified local strain amplification near micromechanical heterogeneities in the brain, including sulci, blood vessels, and axons as possible contributory factors leading to blast-related brain injury. Simulation studies indicate that pressure gradients in the brain of an unhelmeted head resulting from military blast exposure may be sufficiently large to generate damaging intracranial forces, even in the absence of direct impact trauma to the head. Ultrastructural analysis indicates that blast exposure in our experimental model was associated with persistent microvascular pathology, including abnormal blood-brain bather (BBB) cytoarchitecture. Blast-related ultrastructural pathology may be associated with pericyte degeneration and/or microvascular compression secondary to astrocytic end-feet swelling, thereby leading to BBB compromise, local hypoxia, chronic neuroinflammation, and neurodegeneration.

The significance of the neurophysiological abnormalities in blast-exposed wild-type C57BL/6 mice is substantial. First, although blast exposure did not produce detectable long-term dysfunction in basal synaptic transmission, exposure to a single sublethal blast was sufficient to induce profound and persistent impairment of both activity- and cAMP-dependent LTP in hippocampal CA1 pyramidal neurons, candidate cellular mechanisms of long-term memory processing. The fact that both forms of LTP require dendritic protein synthesis and gene transcription indicate that blast exposure may induce long-lasting damage to cellular signal transduction downstream of synaptic glutamate release. Mechanisms that may be altered by blast exposure include N-methyl-_{D}-aspartate glutamate receptor activation, intracellular second messenger systems, gene expression, protein synthesis, and posttranslational modification. Our results also indicate that blast exposure can induce persistent axonal conduction defects that further impair cognitive processing and are consistent with recent findings from human studies. These effects may be mediated by diffuse axonal injury, Wallerian degeneration, and/or differential susceptibility of larger neurons to structural or functional axotomy. Damage to these and other brain structures, systems, and mechanisms may contribute to abnormalities in neurochemical homeostasis, cerebral metabolism, and neurophysiological functions associated with blast-related TBI. Our results suggest that blast exposure holds comparable or even greater pathogenic potential than repetitive head injury associated with contact athletics.

Our results provide compelling evidence linking blast exposure to long-lasting brain injury. Specifically, our data indicate that blast exposure increases risk for later development of CTE and associated neurobehavioral sequelae. Indeed, the severity, persistence, and possible progression of the neuropathological abnormalities and neurophysiological deficits observed in our study indicate that blast exposure is a potent insult with enduring pathogenic potential and functional significance. The neuropathologically validated murine model with correspondence to human CTE is a useful tool to evaluate mechanisms, biomarkers, and risk factors relevant to blast-related brain injury and facilitate development of diagnostics, therapeutics, and prophylactic measures for blast neurotrauma and its aftermath.

### TBI-CTE Blood Biomarkers

Traumatic brain injury (TBI) is the “signature injury” of the conflicts in Iraq and Afghanistan. Department of Defense investigators have reported that 15.8% of a large cohort of wounded U.S. troops injured during military combat in Iraq sustained a TBI. Of these TBIs, 89.3% were classified as mild (mTBI) and nearly all (96%) were associated with blast exposure. Cumulative statistics (2000 to 2012) compiled by the Armed Forces Health Surveillance Center indicate that a total of 266,810 troops sustained a TBI of which 82.4% were classified as mTBI. An estimated 19.5% of troops returning from Iraq and Afghanistan experienced TBI during deployment. However, the studies may underestimate the number of troops with TBI, especially in combat soldiers exposed to blast from improvised explosive devices (IEDs). The Defense and Veterans Brain Injury Center (DVBIC) has reported that 59% of blast-exposed troops sustained a TBI. It is generally recognized that between a quarter million to more a half million troops may have experienced a deployment-related TBI.

Despite growing public awareness of TBI, veterans in need are not receiving medical care for this condition. Moreover, emerging evidence indicates that TBI may trigger later development of serious neurological sequelae including a devastating tau protein-linked neurodegenerative disease known as chronic traumatic encephalopathy (CTE). TBI can be a pathogenic trigger for later development of CTE in athletes engaged in contact sports and military service personnel exposed to explosive blast. Mechanistic links between acute blast neurotrauma and chronic neurological sequelae including CTE have been demonstrated in a mouse model that recapitulates clinical features of the human disease.

Blast TBI is associated with injury to brain cells (neurons, glia), structures (axon fiber tracts, blood-brain barrier), and functions (axonal conduction, synaptic plasticity) that may lead to persistent cognitive deficits, executive dysfunction, and long-term neuropsychiatric disability. Clinical syndromes associated with blast exposure include post-traumatic stress disorder (PTSD), post-concussion syndrome (PCS), and CTE and its variants. In the military setting, these conditions may impair operational judgment, compromise personnel safety, and undermine mission objectives. TBI-related neurobehavioral deficits may also increase risk of injury to self (e.g., impulsive behavior, suicide) and others (e.g., assaultive behavior, homicide). Prior to the invention, there were no methods to diagnose, prevent, treat, or monitor TBI or CTE in living people.

### Tau Structure and Sequences

Tau is the major neuronal microtubule-associated protein. The human tau gene is located on the long arm of chromosome 17 (position 17q21) and contains 16 exons. Three of these exons (exons 4A, 6, and 8) are present only in mRNA of peripheral tissue and are never present in mRNA of the human brain. Exons-1 and 14 are transcribed but not translated. Exons 2, 3, and 10 are alternatively spliced, and exon 3 never appears in the absence of exon 2. Hence, the alternative splicing of these three exons produces six isoforms of tau in adult brain (FIG. 39). The six isoforms of tau differ from each other by the presence or absence of one or two inserts (29 or 58 amino acids) in the N-terminal part and by the presence of either three or four repeats in the C-terminal region. The region upstream of the microtubule binding domains contains many proline residues and, hence, is called the proline-rich region. NCBI Reference Sequences are provided below.

Tau (microtubule-associated protein tau)
Isoform 2 [Homo sapiens]

### NP_{—}005901.2

441 aa

### 2N/4R

```
(SEQ ID NO: 1)
    1 maeprqefev medhagtygl gdrkdqggyt mhqdgegdtd
        aglkesplqt ptedgseepg
  61 setsdakstp taedvtaplv degapgkqaa aqphteipeg
        ttaeeagigd tpsledeaag
121 hvtqarmvsk skdgtgsddk kakgadgktk iatprgaapp
        gqkgqanatr ipaktppapk
181 tppssgeppk sgdrsgyssp gspgtpgsrs rtpslptppt
        repkkvavvr tppkspssak
241 srlqtapvpm pdlknvkski gstenlkhqp gggkvqiink
        kldlsnvqsk cgskdnikhv
301 pgggsvqivy kpvdlskvts kcgslgnihh kpgggqvevk
        sekldfkdrv qskigsldni
361 thvpgggnkk iethkltfre nakaktdhga eivykspvvs
        gdtsprhlsn vsstgsidmv
421 dspqlatlad evsaslakqg l
```

Tau (microtubule-associated protein tau)
Isoform 8 [Homo sapiens]

### NP_{—}001190181.1

410 aa

### 2N/3R

```
(SEQ ID NO: 2)
    1 maeprqefev medhagtygl gdrkdqggyt mhqdgegdtd
        aglkesplqt ptedgseepg
  61 setsdakstp taedvtaplv degapgkqaa aqphteipeg
        ttaeeagigd tpsledeaag
121 hvtqarmvsk skdgtgsddk kakgadgktk iatprgaapp
        gqkgqanatr ipaktppapk
181 tppssgeppk sgdrsgyssp gspgtpgsrs rtpslptppt
        repkkvavvr tppkspssak
241 srlqtapvpm pdlknvkski gstenlkhqp gggkvqivyk
        pvdlskvtsk cgslgnihhk
301 pgggqvevks ekldfkdrvq skigsldnit hvpgggnkki
        ethkltfren akaktdhgae
361 ivykspvvsg dtsprhlsnv sstgsidmvd spqlatlade
        vsaslakqg l
```

Tau (microtubule-associated protein tau)
Isoform 5 [Homo sapiens]

### NP_{—}001116539.1

412 aa

### 1N/4R

```
(SEQ ID NO: 3)
    1 maeprqefev medhagtygl gdrkdqggyt mhqdgegdtd
        aglkesplqt ptedgseepg
  61 setsdakstp taeaeeagig dtpsledeaa ghvtqarmvs
        kskdgtgsdd kkakgadgkt
121 kiatprgaap pgqkgqanat ripaktppap ktppssgepp
        ksgdrsgyss pgspgtpgsr
181 srtpslptpp trepkkvavv rtppkspssa ksrlqtapvp
        mpdlknvksk igstenlkhq
241 pgggkvqiin kkldlsnvqs kcgskdnikh vpgggsvqiv
        ykpvdlskvt skcgslgnih
301 hkpgggqvev ksekldfkdr vqskigsldn ithvpgggnk
        kiethkltfr enakaktdhg
361 aeivykspvv sgdtsprhls nvsstgsidm vdspqlatla
        devsaslakq g l
```

Tau (microtubule-associated protein tau)
Isoform 7 [Homo sapiens]

### NP_{—}001190180.1

381 aa

### 1N/3R

```
(SEQ ID NO: 4)
    1 maeprqefev medhagtygl gdrkdqggyt mhqdgegdtd
        aglkesplqt ptedgseepg
  61 setsdakstp taeaeeagig dtpsledeaa ghvtqarmvs
        kskdgtgsdd kkakgadgkt
121 kiatprgaap pgqkgqanat ripaktppap ktppssgepp
        ksgdrsgyss pgspgtpgsr
181 srtpslptpp trepkkvavv rtppkspssa ksrlqtapvp
        mpdiknyksk igstenikhq
241 pgggkvqivy kpvdlskvts kcgslgnihh kpgggqvevk
        sekldfkdrv qskigsldni
301 thvpgggnkk iethkltfre nakaktdhga eivykspvvs
        gdtsprhlsn vsstgsidmv
361 dspglatlad evsaslakqg l
```

Tau (microtubule-associated protein tau)
Isoform 3 [Homo sapiens]

### NP_{—}058518.1

383 aa

### 0N/4R

```
(SEQ ID NO: 5
    1 maeprqefev medhagtygl gdrkdqggyt mhqdgegdtd
        aglkaeeagi gdtpsledea
  61 aghvtgarmv skskdgtgsd dkkakgadgk tkiatprgaa
        ppgqkgqana tripaktppa
121 pktppssgep pksgdrsgys spgspgtpgs rsrtpslptp
        ptrepkkvav vrtppkspss
181 aksrlqtapv pmpdlknvks kigstenlkh qpgggkvqii
        nkkldlsnvq skcgskdnik
241 hvpgggsvqi vykpvdlskv tskcgslgni hhkpgggqve
        vksekldfkd rvqskigsid
301 nithvpgggn kkiethkltf renakaktdh gaeivykspv
        vsgdtsprhl snvsstgsid
361 mvdspglatl adevsaslak qg l
```

Tau (microtubule-associated protein tau)
Isoform 4 [Homo sapiens]

### NP_{—}058525.1

352 aa

### 0N/3R

```
(SEQ ID NO: 6)
    1 maeprqefev medhagtygl gdrkdqggyt mhqdgegdtd
        aglkaeeagi gdtpsledea
  61 aghvtqarmv skskdgtgsd dkkakgadgk tkiatprgaa
        ppgqkgqana tripaktppa
121 pktppssgep pksgdrsgys spgspgtpgs rsrtpslptp
        ptrepkkvav vrtppkspss
181 aksrlqtapv pmpdlknvks kigstenlkh qpgggkvgiv
        ykpvdlskvt skcgslgnih
241 hkpgggqvev ksekldfkdr vqskigsldn ithvpgggnk
        kiethkltfr enakaktdhg
301 aeivykspvv sgdtsprhls nvsstgsidm vdspqlatla
        devsaslakq g l
```

### Blood-Based CTE Biomarkers and Sequences

The following biomarkers are used alone or together with measurements of Tau proteins, isoforms, and fragments thereof to calculate prognosis for developing CTE after a traumatic brain insult.

Other Blood-Based TBI-CTE Biomakers are prioritized below.

1. αB-Crystallin—astrocytosis
αB-Crystallin [Homo sapiens]

### GenBank: ACP18852.1

175 aa

```
(SEQ ID NO: 7)
    1 mdiaihhpwi hrpffpfhsp srlfdqffge hllesdlfpt
        stslspfylr ppsflrapsw
  61 fdtglsemrl ekdrfsvnld vkhfspeelk vkvlgdviev
        hgkheerqde hgfisrefhr
121 kyripadvdp ltitsslssd gvltvngprk qvsgpertip
        itreekpavt aapkk
```

2. Chemokine (C-C motif) ligand 2 [Homo sapiens]
Formerly known as Monocyte chemoattractant protein-1 (MCP-1)

### GenBank: AAH09716.1

99 aa

```
(SEQ ID NO: 8)
  1 mkvsaallcl lliaatfipq glaqpdaina pvtccynftn
      rkisvqrlas yrritsskcp
61 keavifktiv akeicadpkq kwygdsmdhl dkqtqtpkt
```

2a. Chemokine (C-C motif) Ligand 2, Isoform CRA_A [Homo sapiens]

### GenBank: EAW80211.1

65 aa

```
(SEQ ID NO: 9)
  1 mkvsaallcl lliaatfipq glaqpdaina pvtccynftn
      rkisvqrlas yrritsskcp
61 keavm
```

2b. 3. Chemokine (C-C motif) Ligand 2, Isoform CRA_B [Homo sapiens]

### GenBank: EAW80212.1

99 aa

```
(SEQ ID NO: 10)
  1 mkvsaallcl lliaatfipq glaqpdaina pvtccynftn
      rkisvqrlas yrritsskcp
61 keavifktiv akeicadpkq kwygdsmdhl dkqtqtpkt
```

3. Ubiquitin C-terminal hydrolase (UCH-L1)—neuronal injury
Ubiquitin carboxyl-terminal hydrolase isozyme L1 [Homo sapiens]

### NP_{—}004172.2

223 aa

```
(SEQ ID NO: 11)
    1 mqlkpmeinp emlnkvlsrl gvagqwrfvd vlgleeeslg
        svpapacall llfpltaghe
  61 nfrkkqieel kgqevspkvy fmkqtignsc gtiglihava
        nnqdklgfed gsvlkqflse
121 tekmspedra kcfekneaiq aahdavaqeg qcrvddkvnf
        hfilfnnvdg hlyeldgrmp
181 fpvnhgasse dtllkdaakv creftereqg evrfsavalc
        kaa
```

4. Glial Fibrillary Acidic Protein (GFAP)—astrocytosis
Glial Fibrillary Acidic Protein [Homo sapiens]

### GenBank: AAB22581.1

432 aa

```
(SEQ ID NO: 12)
    1 merrritsaa rrsyvssgem mvgglapgrr lgpgtrlsla
        rmppplptrv dfslagalna
  61 gfketraser aemmelndrf asyiekvrfl eqqnkalaae
        lnqlrakept kladvyqael
121 relrlrldql tansarleve rdnlaqdlat vrqklqdetn
        lrleaennla ayrqeadeat
181 larldlerki esleeeirfl rkiheeevre lgeglarqqv
        hveldvakpd ltaalkeirt
241 qyeamassnm heaeewyrsk fadltdaaar naellrqakh
        eandyrrqlq sltcdleslr
301 gtneslerqm reqeerhvre aasyqealar leeegqslkd
        emarhlgeyq dllnvklald
361 ieiatyrkll egeenritip vqtfsnlqir etsldtksvs
        eghlkrnivv ktvemrdgev
421 ikeskqehkd vm
```

### Adjunctive Blood-Based TBI Targets

### S100-β [Homo Sapiens]

### GenBank: AAH01766.1

92 aa

### Neuron-Specific Enolase (NSE), Gamma-Enolase [Homo Sapiens]

### NP_{—}001966.1

434 aa

### Interleukin-8 (IL-8)

### GenBank: AAH13615.1

99 aa
Interleukin-6 (Interferon, Beta-2) [Homo sapiens]

### GenBank: AAH15511.1

212 aa
Myelin Basic Protein (MBP) and fragments/isoforms (many) [Homo Sapiens]

### UniProtKB/Swiss-Prot: P02686.3

304 aa

### αII-Spectrin Breakdown Products (αII-SBDP)

Note: large series of these peptides and proteins
Full protein: GenBank: AAB41498.1; 2477 aa
For example, 150 kDa (SBDP150) and 145 kDa (SBDP145) by calpain, 120-kDa product (SBDP120) by caspase-3

Non-brain restricted targets such as interleukin-6 or 8 are useful in prognostic signatures together with tau/p-tau to further increase clinical confidence regarding prognosis. Increased levels above normal values further indicate a poor prognosis.

### Detection of Prognostic Biomarkers in Bodily Fluids

In addition to laboratory methods such as mass spectroscopy and ELISA, a ruggedized, field-deployable handheld device that provides rapid and reliable assessment of acute neurological injury using a single drop of blood (5 μL) drawn by finger prick is used to detect biomarker levels in an acute situation, e.g., minutes, hours, days after an incident, or after longer periods of time after a potential brain injury. The diagnostic/prognostic technology provides quantitative clinical information regarding degree of acute neurological injury (e.g., TBI), and equally important, potential for chronic neuropsychiatric and/or cognitive impairment. Specifically, the diagnostic platform is designed to enable analytical assessment of set of blood-based biomarkers indicative of acute brain injury and predictive of neurologic sequelae and chronic neurocognitive impairment. Conversely, this platform is useful to objectively triage individuals to an appropriate level of medical care or discharge to outpatient follow-up. The point-of-care diagnostic platform is compatible with systems and methods in neurotraumatology, experimental neurology, and protein microanalysis and is useful for a broad range of military and civilian medical applications.

### Primary Blood-Based TBI Biomarker Targets

Each of the following protein biomarkers indicated conjunction of trauma-induced brain damage and breakdown of the blood-brain barrier (BBB) that allows passage of the index brain-derived proteins into the peripheral circulation. Identification of these biomarkers in the peripheral circulation are thus indicative of organic brain injury, and in the setting of concordant clinical findings, increased risk of chronic neurological sequelae, including CTE.

### Neuronal Injury and Axonopathy

Total tau protein (T-Tau), modified tau protein and breakdown products (C-Tau, P-Tau, G-Tau, BD-Tau), and/or UCH-L1 are used as prognostic biomarkers.

Microtubule-associated protein tau (MAPT, tau) is a neuron-specific protein that localizes to the axonal compartment of neurons. Tau is expressed as multiple isoforms and is subject to extensive post-translational processing. Pathological hyperphosphorylation and glycation promotes tau aggregation and formation of neurofibrillary tangles, cardinal neuropathological hallmarks of Alzheimer's disease and various tauopathies, including CTE. Concentrations of total tau protein in cerebrospinal fluid (CSF) increase after acute TBI and correlate with severity of axonal trauma. Elevated serum total tau levels reportedly correlate with trauma severity in human patients with TBI. Laboratory studies conducted in rats demonstrated that total tau levels rise quickly after TBI (>3 fold increase within 1 hour), decline after 6 hours, and return to baseline within 24 hours. There is a positive correlation of serum total tau with trauma severity. Increased total tau protein levels in CSF obtained from elite amateur boxers have been detected following both acute and chronic (repetitive) head trauma. Plasma tau levels were elevated in Olympic boxers from whom blood samples were obtained 1-6 days after a bout. Analysis of a second blood draw obtained after a two-week rest period indicated that plasma tau levels dropped significantly but remained elevated relative to control levels. An ultra-sensitive digital array assay system has been used to detect serum total tau (non-phosphorylated and phosphorylated species) secondary to hypoxic brain injury in patients with cardiac arrest. Elevated serum tau levels ranged from modest (<10 pg/mL) to very high (˜700 pg/mL). In many patients, the serum tau levels exhibited bimodal kinetics in which early tau elevations appeared within 24 hours after cardiac arrest and a second delayed peak after 24-48 hours. In patients with delayed serum tau elevations, serum tau concentration was highly predictive of 6-month neurological outcome. Conversely, patients who exhibited minimal serum tau (<1 pg/mL) across the sampling interval demonstrated good clinical outcomes. Analysis of fractionated tau products, especially phosphorylated species, yields additional information regarding the evolution of acute neurotrauma as well as the extent and course of secondary injury, thus extending clinical utility beyond the acute phase of recovery following TBI.

Total tau protein (plasma): >2 SD above normal control values (e.g., ˜1 pg/mL). Increased blood levels of phosphorylated tau (p-tau) and/or other tau isoforms (c-tau, g-tau, bd-tau, etc.) reflects the extent and spectrum of diffuse axonopathy resulting from ongoing neuronal injury or initiation of secondary injury. Normative values may vary as a function of specimen collection, storage, analytical method, specifics of the index metric (e.g., total protein, fractionated isoform, post-translational modifications, breakdown products), and composition and size of the normative control population.

Ubiquitin C-terminal hydrolase (UCH-L1) is a neuron-specific cytoplasmic enzyme involved in processing ubiquitinated proteins that are destined to be metabolized via the ATP-dependent proteasome pathway. Increased CSF and blood concentrations of UCH-L1 have been associated with neuron destruction and increased blood-brain barrier (BBB) permeability. UCH-L1 concentrations are also elevated in other neurological diseases marked by neuronal injury, including stroke, aneurysmal subarachnoid hemorrhage, and neonatal hypoxic-ischemic encephalopathy. After TBI, blood UCH-L1 levels correlate with injury severity and outcome at discharge 6 months after injury.

UCH-L1 (plasma): >2 SD above normal control values (e.g., 0.15 ng/mL). Normative values may vary as a function of specimen collection, storage, analytical method, specifics of the index metric (e.g., total protein, fractionated isoform, post-translational modifications, breakdown products, etc) as well as the composition and size of the assessed control population.

Astrocytosis is identified by measuring and computing Glial fibrillary acidic protein (GFAP), αB-crystallin levels. Glial fibrillary acidic protein (GFAP) is a component of the astrocytic cytoskeleton. Elevated blood concentrations of this brain-specific biomarker have been reported in serum following acute TBI. GFAP is elevated in serum within 4 hours after mild TBI. αB-crystallin, a prototypic small heat shock protein and molecular chaperone, is expressed by and exosomally secreted from activated astrocytes in the brain. Detection of elevated levels of GFAP and αB-crystallin in the blood is indicative of activated astrocytosis and damage to the blood-brain barrier (BBB), both conditions that reflect neurological injury associated with acute TBI.

GFAP and/or breakdown products (plasma) at levels of >2 SD above normal control values (e.g., 250 ng/L) indicate a poor prognosis and predict CTE. Normative values may vary as a function of specimen collection, storage, analytical method, specifics of the index metric (e.g., total protein, fractionated isoform, post-translational modifications, breakdown products, etc) as well as the composition and size of the assessed control population.

All of the markers discussed above are indicative of Blood-Brain Barrier damage:

### Neuroinflammatory Recruitment: CCL2 (MCP-1)

Monocyte chemoattractant protein-1 (MCP-1, now CCL2) is produced by astrocytes within hours after injury. CCL2 levels correlate with the amount of recruited macrophages and severity and extent of traumatic injury. CCL2 is released as an autocrine mediator by infiltrating macrophages and microglia, thus perpetuating peripheral monocyte migration into the brain as a consequence of ongoing secondary injury. CCL2 overexpression in animal models has been shown to increase macrophage infiltration and neurological deficits following ischemia whereas deletion attenuates infiltratration, neuropathology, and neurobehavioral deficits in animal models of traumatic brain injury, stroke, and multiple sclerosis. CCL2 levels in CSF samples rapidly increased following TBI and remained elevated for days.

### Adjunctive Blood-Based TBI Targets: S100-13, Neuron-Specific Enolase (NSE), Interleukin-8 (IL-8), Interleukin-6 (IL-6), Myelin Basic Protein (MBP), Spectrin Breakdown Products

These biomarkers comprise a set of distinct brain-derived proteins with differential cellular specificity, localization, and function. Blood-based assessment of these biomarkers (along with their respective breakdown species and post-translationally-modified products) provide a peripherally-accessible molecular fingerprint that reflects the degree and spectrum of neuronal injury, BBB dysfunction, and neuroinflammation associated with acute brain injury. Detection of fractionated species of phosphorylated tau protein and the neuroinflammatory peripheral monocyte recruitment molecule CCL2 (MCP-1) provide additional clinically-relevant information indicative of evolving neuronal injury, secondary injury, and potential for chronic neurological sequelae. Sensitivity, specificity, and clinical utility of the developed blood-based diagnostic platform is enhanced by simultaneous analysis of multiple biomarkers and replicate sampling across multiple time points (serial assessment). Diagnostic and prognostic power utilizing the developed platform is further facilitated by integration with an evidence-based algorithm that incorporates trauma information (e.g., blast intensity, time since incident, evidence of polytrauma), clinical data (e.g., vital signs, including pulse oximetry), and neurological examination results (e.g., mental status, sensorimotor deficits, psychomotor reactivity). Clinical metrics that are optionally integrated into a diagnostic algorithm include: Glasgow Coma Scale assessment, sensorimotor evaluation, pupillary reflexes, visual tracking, dichotic auditory testing, and psychometric testing. Results of radiological examination provide additional relevant information if available. Clinical implementation of the proposed diagnostic platform for assessment of acute TBI is based on analogy to accepted emergency medical practice for workup and differential diagnosis of chest pain in the setting of presumptive acute myocardial infarction.

The markers are evaluated and computed to yield a prognosis for CTE. Exemplary methods are described below.

Sample Collection & Preparation.

A blood-based specimen for analysis is prepared from fresh whole blood as either serum or plasma using conventional techniques. For reasons described below, the preferred specimen for analytical assessment is platelet-depleted plasma. A fresh blood sample is drawn from a venous, areterial, or capillary source by antecubital venipuncture, arterial line sampling, finger prick, or other blood-sampling technique. The volume of blood drawn for analysis depends on the analytical and collection method chosen. For venous and arterial samples, samples may be acquired in conventional vacutainer tubes (4.5 ml) filled to within 10% of capacity. All non-gel blood collection tubes, including those that contain heparin, EDTA and non-gel serum tubes can be centrifuged at ≦1300 RCF for 10 minutes. Blood collection following lancet finger prick may utilize a suitable microfuge container, capillary tube, absorbent blotting material, or adsorbent matrix.

Serum Preparation.

Preparation of serum specimens are prepared by allowing a freshly drawn blood sample to rest at room temperature for a clotting time between 30 min to 60 min. Serum samples prepared by clotting times of 30 min or less are expected to retain cellular components and other elements that may affect analysis. Samples prepared with clotting times greater than 60 min may result in cell lysis, thus releasing cellular proteins that are not normally detected in serum.

Plasma Preparation.

By contrast, plasma sampling is less time-consuming and yields a more reliable specimen preparation with greater volume compared to serum. Moreover, plasma preparations are generally more stable than serum. Although either biospecimen preparation may be utilized, the preferred enablement favors plasma preparation using conventional anticoagulants (with target concentrations) in the following rank order of preference: EDTA (˜1.3 mmol/L)>>sodium heparin (1.30 mmol/L)=lithium heparin (1.33 mmol/L)>sodium citrate (1.09 mmol/L). Platelet contamination and activation are responsible for the release of platelet-related peptides in plasma samples that may contribute to artifact biomarker signals. Thus, the preferred method of plasma preparation includes a gentle platelet removal step (i.e., total platelet count <10/nL) using either a low protein-binding sterile filter (0.2 mm) after the first round of centrifugation, or alternatively, sequential centrifugation (2500×g for 15 min) at room temperature.

Analytical Assessment.

Mass spectrometry (MS) is s preferred technology for analysis Aebersold et al., 2003, Nature 422: 198-207; Lista et al., 2013, Progress in Neurobiology 101-102: 18-34). Mass spectrometric measurements are performed in the gas phase on ionized analytes. Matrix-assisted laser desorption/ionization time-of-flight (MALDI-TOF) is generally employed to volatize and ionize the target proteins and peptides of interest. MS analysis can be coupled with other protein analytical techniques to provide additional quantitative information. Two-dimensional gel polyacrylamide electrophoresis (2D-PAGE) separates proteins according to charge and size in two individual steps: isoelectric focusing (IEF) and sodium dodecyl sulphate polyacrylamide gel electrophoresis (SDS-PAGE). This separation leads to detect a pattern of protein spots whose identities are revealed using MS methods, thus providing protein identification and quantitative information of a biomarker (Gorg et al., 2004, Proteomics 4, 3665-3685; Hye et al., 2006, Brain 129: 3042-3050). Liquid chromatography (LC) process is a method for the fractionation of proteins characterized by mass-transfer between a stationary and a liquid mobile phase. High pressure conditions are habitually employed to move the analytes along a chromatographic column (HPLC, high-performance liquid chromatography). The combination of LC with MS or tandem mass spectrometry (MS/MS) permits identification of peptides in mixtures in a single analysis and provided an increased potential to investigate low abundance proteins (Domon et al., 2006, Science 312: 212-217; Hye et al., 2006; Drabik et al., 2007, Mass Spectrometry Reviews 26: 432-450; Liao et al., 2007 Proteomics Clinical Applications 1: 506-512; Cutler et al., 2008, Proteomics Clinical Applications 2: 467-477; Thambisetty et al., 2010, Biomarkers in Medicine 4, 65-79; Thambisetty et al., 2010b, Archives of General Psychiatry 67: 739-748). Surface enhanced laser desorption/ionization time-of-flight (SELDI-TOF), a MALDI-TOF variant, utilizes protein chip array for selective capture of proteins and can be utilized for blood-based biomarker analysis (Hutchens et al., 1993, Rapid Communications in Mass Spectrometry 7: 576-580; Issaq et al., 2007, Chemical Reviews 107: 3601-3620; Merchant and Weinberger, 2000, Electrophoresis 21: 1164-1177).

Single-molecule enzyme-linked immunosorbent assay (ELISA) for target biomarkers in serum or plasma can be accomplished using antibody-mediated capture to microscopic beads or adsorbent matrices to detect low-abundance serum or plasma proteins at subfemtomolar concentrations. Such a system can be used to detect total tau protein in serum or plasma samples when optimally coupled with a suitable monoclonal capture (e.g., Tau5, Covance, Princeton, N.J., USA) and detection antibodies (e.g., HT7 and BT2, Pierce Biotechnology, Rockford, Ill., USA) (Randall et al., 2013, Resuscitation 84: 351-356; Neselius et al., 2013, Brain Injury, 1-9).

### Detection and Computation of MAPT/Tau Level for CTE Prognosis

As described above, MAPT, tau is a neuron-specific protein that localizes to the axonal compartment of neurons. Tau is expressed as multiple isoforms and is subject to extensive post-translational processing. Pathological hyperphosphorylation and glycation promotes tau aggregation and formation of neurofibrillary tangles, cardinal neuropathological hallmarks of Alzheimer's disease and various tauopathies, including CTE. The data described herein indicates that that analysis of fractionated tau products, especially phosphorylated species, yields valuable information regarding the evolution of acute neurotrauma as well as the extent and course of secondary injury, thus extending clinical utility beyond the acute phase of recovery following TBI and permitting physicians to make a prognosis regarding whether a patient is likely to progress to CTE.

Total Tau Protein and Phosphorylated Tau Protein (Plasma or Serum).

Enhanced ELISA detection using a suitable monoclonal capture (e.g., Tau5, Covance, Princeton, N.J., USA) and detection antibodies (e.g., HT7 and BT2, Pierce Biotechnology, Rockford, Ill., USAquantitative MS analysis, or other methodology described above indicates poor prognosis levels are >2 SD above normal control values (e.g., ˜2 pg/mL. Expected normal range: 0.5+1 pg/ml. Normative values may vary as a function of specimen collection, storage, analytical method, specifics of the index metric (e.g., total protein, fractionated isoform, post-translational modifications, breakdown products), and composition and size of the normative control population.

Increased blood levels of phosphorylated tau (p-tau) reflects the extent, spectrum, and duration of diffuse axonopathy resulting from ongoing neuronal injury, initiation of secondary injury, and/or progression of axonopathy. Thus, the presence and level of these biomarkers in the blood, either plasma or serum, correlate with increasingly poor prognostic outcome above and beyond the total tau signature alone. Clinically-validated normative control values have not been reported for phosphorylated tau protein (p-tau). Under normal conditions, levels should be at or below analytical detection limit. Preferred antibodies include CP13 (or any antibody or ligand specific for 0202); PFH-1 (or any antibody or ligand specific for pS396, 0404); AT8 (or any antibody or ligand specific for pS202, pT205; Pierce Biotechnology, Rockford, Ill., USA); AT270 (or any antibody or ligand specific for pT181; Innogenetics, Alpharetta, Ga., USA). Normative values may vary as a function of specimen collection, storage, analytical method, specifics of the index metric (e.g., total protein, fractionated isoform, post-translational modifications, breakdown products), and composition and size of the normative control population.

The temporal dynamics of Tau levels is analogous to that of cardiac enzymes after a heart attack. The observed bimodal elevation kinetics are consistent with two modes of neuronal damage: initially upon acute oxygen deprivation, followed by delayed cell death due to secondary injury. Area under curve (AUC) is useful as an index metric for serial sample analysis. (Randall et al., 2013, Resuscitation 84: 351-356).

Blood-Based (Plasma or Serum) Signatures Indicative of Poor Prognosis and/or Increased Risk of Significant Neurological Sequelae, Including CTE

Tau and other biomarker signatures derived from patient bodily fluid such as plasma or serum are ranked in order below in terms of increasingly poor prognosis, increased risk of developing CTE following an acute brain injury or insult from TBI.
Elevated total tau protein>normal tau protein levels
Presence of phosphorylated tau protein
Presence of phosphorylated tau protein in combination with elevated total tau protein (levels in Tau document sent under separate email cover)
Increasing levels of total or phosphorylated tau protein on sequential samples (hours to days)
Chronic elevation of total or phosphorylated tau protein on sequential samples (weeks to years)
Temporally increasing ratio of total to phosphorylated tau protein over any time period (hours to years). If both total tau and phospho-tau are detectable, this ratio is tracked and monitored similar to what is done for cardiac enzymes following a heart attack. The reference values are those obtained earlier in time from the same patient.
Confidence level of any of the above is enhanced by concordant elevation of one or more target biomarkers that reflect ongoing neuronal injury (UCHL1), astrocytosis (alphaB-crystallin, GFAP), or neuroinflammation (CCL2).

### Evaluation of Ocular Tissues and Function

Analysis of the eye and ocular tissues is useful as an adjuctive test to confirm diagnostic and prognostic determinations based on biomarkers. Adult male C57BL/6 mice were subjected to single blast or sham blast (no blast control) as described above. Two weeks following single-blast or sham-blast exposure, the mice were either: (i) sacrificed and the brains and eyes harvested for routine histopathology (hematoxylin and eosin staining, FIG. 41A-B), or (ii) assessed by in vivo full-field electroretinography (FIG. 41C) with data analysis of pertinent ERG waves presented as mathematical models (FIGS. 41D-E). FIG. 41C shows representative responses obtained from the dark adapted eye of a control and blasted mouse using a six log unit range of stimuli. The top right panel (FIG. 41D) shows the first 20 ms, mostly the a-waves, of the responses to the eight brightest stimuli (grey lines). The Hood and Birch formulation of the Lamb and Pugh model of the activation of phototransduction (colored lines) is fitted to the first 8 ms of these responses (black lines). The a-waves are smaller in the blast-exposed mice. The trough-to-peak amplitudes of the b-wave in the response to the 14 dimmest stimuli are plotted in the bottom right panel (FIG. 41E). The Naka-Rushton equation is fitted through the data. The b-waves are also much smaller in the blast-exposed mice. The oscillatory potentials (OPs), periodic wavelets superimposed on the leading edge of the b-wave at higher intensities, are also smaller and slower in the blast-exposed mice. These results indicate that both photoreceptor and postreceptor retinal responses are dysfunctional in mice exposed to blast compared to control mice. In the context of findings showing that blast and impact neurotrauma are functional identical in terms of brain pathology and functional sequelae, the same outcome applies in impact neurotrauma.

### Detection Platforms

The selected set of TBI biomarkers comprise a set of distinct brain-derived proteins with differential cellular specificity, localization, and function. Blood-based assessment of these biomarkers (along with their respective breakdown species and post-translationally-modified products) are detected using a field-deployable, point-of-care instrument that analytically evaluates whole blood, plasma, serum, or blood-based fraction obtained by venipuncture, arterial sampling, finger prick, or other method of blood draw. Analytical assessment of the target biomarkers provides a peripherally-accessible molecular fingerprint that reflects the presence, intensity, spectrum, and evolution of neuronal injury, BBB dysfunction, and neuroinflammation associated with acute brain injury as well as prognostic information relevant to assessment of clinical course and risk of long-term neurological and neurobehavioral sequelae, including CTE and variant disorders. Detection of fractionated species of phosphorylated tau protein and the neuroinflammatory peripheral monocyte recruitment molecule CCL2 (MCP-1) provides additional clinically-relevant information indicative of evolving neuronal injury, secondary injury, and potential for chronic neurological sequelae. Sensitivity, specificity, and clinical utility of the developed blood-based diagnostic platform is enhanced by simultaneous analysis of multiple biomarkers and replicate sampling across multiple time points (serial assessment). Diagnostic and prognostic power utilizing the developed platform is further facilitated by integration with an evidence-based algorithm that incorporate trauma information (e.g., traumatic intensity and kinematics, time since incident, evidence of polytrauma, single versus repeated trauma), clinical data (e.g., vital signs, including pulse oximetry), and neurological examination results (e.g., mental status, sensorimotor deficits, psychomotor reactivity). Pertinent clinical metrics are optionally integrated into a diagnostic algorithm include: Glasgow Coma Scale assessment, sensorimotor evaluation, pupillary reflexes, visual tracking, dichotic auditory testing, and psychometric testing. Results of radiological examination provides additional relevant information if available.

Reagents, e.g., and antibody specific for Tau and/or an epitope containing a phosphorylated residue of Tau (or specific for any of the other markers such as αB Crystallin, GFAP, CCL2) for carrying out the diagnostic or prognostic assay may be packaged together as a kit. For example, the antibody is immobilized on a solid phase and packaged together with other reagents suitable for detecting antibody/antigen complexes. For example, enzyme-conjugated reagents may be included; purified Tau, pTau, or one or more of the other biomarkers may also be included as a standard or control reagent. The solid phase component of the kit onto which an antibody or antigen is immobilized is preferably an assay plate, an assay well, a nitrocellulose membrane, a bead, a dipstick, or a component of an elution column. For example, a capture antibody is immobilized and a secondary antibody is used to detect the immune complex. The kit may also contain a second antibody or other detectable marker. The second antibody or marker is labeled, e.g., using a radioisotope, fluorochrome, or other means of detection.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of determining risk of developing chronic traumatic encephalopathy (CTE) of a subject, comprising detecting a CTE-linked neuropathic marker, said marker comprising a microtubule associated tau protein (Tau) or a fragment thereof, in a bodily fluid after at least a first blast injury, subconcussive injury, acute concussive or subconcussive head injury from blast exposure, impact head injury, acceleration or deceleration head trauma, or closed-skull neurotrauma, wherein a concentration of greater than 0.5±1 pg/ml of the total Tau protein or fragment thereof in the bodily fluid indicates an increased risk of developing CTE, a concentration of greater than 1 pg/ml of the total Tau protein or fragment thereof in the bodily fluid indicates a moderate risk of developing CTE, and a concentration of greater than 5 pg/ml of the total Tau protein or fragment thereof in the bodily fluid indicates a severe risk of developing CTE.

2. The method of claim 1, wherein said bodily fluid comprises a blood composition.

3. The method of claim 1, wherein said blood composition comprises plasma or serum.

4. The method of claim 1, wherein said bodily fluid comprises saliva, urine, whole blood, or cerebrospinal fluid.

5. The method of claim 1, wherein said Tau protein or fragment thereof comprises a phosphorylated amino acid.

6. The method of claim 5, wherein said Tau protein or fragment thereof comprises a phosphorylated amino acid at position S202, S396, S404, T181, or T205.

7-9. (canceled)

10. The method of claim 1, wherein said method further comprises the steps of:
measuring the level of phosphorylated tau and the level of total tau; and
computing a ratio of phosphorylated Tau to total Tau,
wherein an increase in said ratio over time indicates an increased risk of developing CTE.

11. The method of claim 1, further comprising detecting in the bodily fluid one or more of: an αB-Crystallin or a fragment thereof; a Chemokine (C-C motif) ligand 2 or a fragment thereof; an Ubiquitin C-terminal hydrolase (UCH-L1) or a fragment thereof; and a Glial Fibrillary Acidic Protein (GFAP) or a fragment thereof.

12. The method of claim 11, wherein a level of UCH-L1 that is greater than 2 SD above a normal control value of about 0.15 ng/mL indicates an increased risk of developing CTE.

13. The method of claim 11, wherein a level of GFAP that is greater than 2 SD above a normal control value of about 250 ng/L indicates an increased risk of developing CTE.

14. The method of claim 1, further comprising detecting in the bodily fluid one or more of: S100-β or a fragment thereof; Neuron-Specific Enolase (NSE) or a fragment thereof; Interleukin-8 (IL-8) or a fragment thereof; Interleukin-6 (Interferon, Beta-2); Myelin Basic Protein (MBP) or a fragment thereof; and αII-Spectrin Breakdown Product (αII-SBDP) or a fragment thereof.

15. The method of claim 1, wherein said CTE-linked marker comprises phosphorylated tauopathy, myelinated axonopathy, microvasculopathy, chronic neuroinflammation, or neurodegeneration in the absence of macroscopic tissue damage or hemorrhage.

16. The method of claim 1, wherein said CTE-linked marker comprises:
a) phosphorylated forms of tau protein or tau protein fragments (tau peptides),
b) biomarkers of myelinated axonopathy; microvasculopathy; or blood-brain barrier compromise or loss of structural or functional integrity of the blood-brain barrier;
c) chronic neuroinflammation and neuroinflammatory mediators, cytokines, and/or peptides;
d) reactive astrocyte and/or microglial products; and/or
e) neurodegeneration in the absence of macroscopic tissue damage or hemorrhage.

17. The method of claim 1, wherein said CTE-linked marker is evaluated at least one week after said blast injury, subconcussive injury, or concussive injury.

18. The method of claim 1, wherein said CTE-linked marker is evaluated at least one month after said blast injury, subconcussive injury, or concussive injury.

19. The method of claim 1, wherein said CTE-linked marker is evaluated at least one year after said blast injury, subconcussive injury, or concussive injury.

20. The method of claim 1, wherein said blast injury comprises an impact injury or exposure to a blast wind.

21. The method of claim 1, wherein said CTE-linked marker is detected by mass spectrometry.

22. The method of claim 1, wherein said CTE-linked marker is detected by an antibody.

23. The method of claim 22, wherein said CTE-linked marker is detected using enzyme-linked immunosorbent assay (ELISA).

24. The method of claim 1, wherein said CTE marker is evaluated by magnetic resonance imaging, diffusion tensor imaging (DTI), positron emission tomography, magnetic resonance imaging and related imaging modalities, magnetic resonance spectroscopy, analysis of cerebrospinal fluid, blood plasma or serum or whole blood.

25-29. (canceled)

30. A mechanical device comprising a field-deployable actuable mechanical device to prevent movement or acceleration of the head relative to the neck, torso, or local environment.

31. The method of claim 1, wherein said method is further to determine an increased risk of long-term neurological or neurobehavioral sequelae, and variant disorders selected from the group consisting of chronic traumatic encephalopathy with motor neuron disease and chronic traumatic encephalopathy with Parkinsonism.

32. The method of claim 1, further comprising psychometric evaluation, visual field testing, visual field tracking, retinal imaging, eletroretinography, electroencephalography, pupillometry, or imaging or spectroscopic analysis of the anterior and posterior chambers of the eye and the tissues comprised therein.

33. The method of claim 1, comprising detecting the CTE-linked neuropathic marker in the bodily fluid within 24 hours of the first blast injury, subconcussive injury, acute concussive or subconcussive head injury from blast exposure, impact head injury, acceleration or deceleration head trauma, or closed-skull neurotrauma.

34. A method of determining risk of developing chronic traumatic encephalopathy (CTE) of a subject, comprising simultaneously detecting two or more CTE-linked neuropathic markers in a bodily fluid of the subject after at least a first blast injury, subconcussive injury, acute concussive or subconcussive head injury from blast exposure, impact head injury, acceleration or deceleration head trauma, or closed-skull neurotrauma,
wherein said markers comprise a microtubule associated tau protein (Tau) or a fragment thereof, αB-Crystallin or a fragment thereof, a Chemokine (C-C motif) ligand 2 or a fragment thereof, an UCH-L1 or a fragment thereof, a GFAP or a fragment thereof, S100-β or a fragment thereof, NSE or a fragment thereof, IL-8 or a fragment thereof, Interleukin-6, MBP or a fragment thereof, or αII-SBDP or a fragment thereof.

35. The method of claim 34, wherein said markers comprise an exosomally secreted protein.

36. A method of determining risk of developing chronic traumatic encephalopathy (CTE) of a subject, comprising:
detecting a CTE-linked neuropathic marker in a bodily fluid taken from the subject at a first time point after at least a first blast injury, subconcussive injury, acute concussive or subconcussive head injury from blast exposure, impact head injury, acceleration or deceleration head trauma, or closed-skull neurotrauma, and
detecting the CTE-linked neuropathic marker in a bodily fluid taken from the subject at a second time point,
wherein a higher level of the marker at the second time point compared to the first time point indicates an increased risk of developing CTE, and
wherein said marker comprises a microtubule associated tau protein (Tau) or a fragment thereof, αB-Crystallin or a fragment thereof, a Chemokine (C-C motif) ligand 2 or a fragment thereof, an UCH-L1 or a fragment thereof, a GFAP or a fragment thereof, S100-β or a fragment thereof, NSE or a fragment thereof, IL-8 or a fragment thereof, Interleukin-6, MBP or a fragment thereof, or αII-SBDP or a fragment thereof.


SEQUENCE LISTING (12 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 175 aa, Homo sapiens [sequence omitted]

SEQ ID NO: 8 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 9 - protein, 65 aa, Homo sapiens
MKVSAALLCLLLIAATFIPQGLAQPDAINAPVTCCYNFTNRKISVQRLASYRRITSSKCPKEAVM

SEQ ID NO: 10 - protein, 99 aa, Homo sapiens; identical to SEQ ID NO: 8

SEQ ID NO: 11 - protein, 223 aa, Homo sapiens [sequence omitted]

SEQ ID NO: 12 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM